SEQUENCE LISTING (25 sequences)

SEQ ID NO: 1 - protein, 407 aa, synthetic construct
RIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 2 - protein, 407 aa, synthetic construct; identical to SEQ ID NO: 1

SEQ ID NO: 3 - protein, 414 aa, synthetic construct
RIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 4 - protein, 1259 aa, synthetic construct
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 5 - protein, 1259 aa, synthetic construct
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGIQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 6 - protein, 1259 aa, synthetic construct
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQHILLPNVIIMGAEPDLFETNSSNISLRNNYMPSNHGFGSIAIVTFSPEYSFRFNDNSINEFIQDPALTLMHELIHSLHGLYGAKGITTTCIITQQQNPLITNRKGINIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGQYKYFKLSNLLNDSIYNISEGYNINNLKVNFRGQNANLNPRIIKPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 7 - protein, 1259 aa, synthetic construct
MPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVGWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKAIIESKYNSYTLEEKNELTNKYDIEQIENELNQKVSIAMNNIDRFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 8 - protein, 1259 aa, synthetic construct
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFVPELLIPTILVFTIKSFLGSSDNKNKVIKAINNALKERDEKWKEVYSFIVSNWMTKINTQFNKRKEQMYQALQNQVNALKTIIEFKYNSYTLEEKKELKNNYDIEQIENELNQKVSIAMNNIDRFLTESSISYLMKLINEVKINKLREYDENVKTYLLDYIIQHGSILGESQQELNSMVIDTLNNSIPFKLSSYTDDKILISYFNKFFKRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 9 - protein, 1184 aa, synthetic construct
SYEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFKDNSMNEFIQDPALTLMHELIHSLHGLYGAKGITTQYTITQQQNPLITNIKGTNIEEFLTFGGTDLNIITSAQYNDIYTNLLADYKKIASKLSKVQVSNPLLNPYKDVFEKKYGLDKDASGIYSVNINKFNDIFKKLYSFTEFDLATKFQVKCRQTYIGQYKYFKLSNLLNNSIYNISEGYNINTLKVNFRGQNTNLNPRIITPLTGRGLVKKIIRFCKNIVFSKGIRKSICIEINNGELFFVASDNSYNDDNINTPKEIDDTVTSNNNYENDLDQVILNFNSESAPGLSDEKLNLTIQNDAYIPKYDSNGTSDIEQHDVNELNVFFYLDAQKVPEGENNIDFTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKTIIESKYNSYTLEEKNELTNKYNIEQIENELNQKVSIAMNNIEIFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 10 - protein, 1259 aa, synthetic construct
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGRILLEELSKANPYLGNDNTPDNQFHIGDASAVEIKFSNGNQSILLPNVIIMGAEPDLFETNSSNISLRNNYMPSNHGFGSIAIVTFSPEYSFRFNDNSMNEFIQDPALTLMHELIHSLHGLYGAKRITTKYTITQQQNPLITNIRGTNIEEFLTFGGTDLNIITSAQYNDIYTNLLADYKKIASKLSKVQVSNPQLNPYKDIFQEKYGLDKNASGIYSVNINKFDDIFKKLYSFTEFDLATKFQVKCRQTYIGQYKYFKLSNLLNNSIYNISEGYNINTLKVNFRGQNTNLNPRIITQLTGRGLVKKIIRFCKNIVFSKGITKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 11 - protein, 1263 aa, synthetic construct
ACEFMPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGRILLEELSKANPYLGNDNTPDNQFHIGDASAVEIKFSNGNQSILLPNVIIMGAEPDLFETNSSNISLRNNYMPSNHGFGSIAIVTFSPEYSFRFNDNSMNEFIQDPALTLMHELIHSLHGLYGAKRITTKYTITQQQNPLITNIRGTNIEEFLTFGGTDLNIITSAQYNDIYTNLLADYKKIASKLSKVQVSNPQLNPYKDIFQEKYGLDKNASGIYSVNINKFDDIFKKLYSFTEFDLATKFQVKCRQTYIGQYKYFKLSNLLNNSIYNISEGYNINTLKVNFRGQNTNLNPRIITQLTGRGLVKKIIRFCKNIVFSKGITKSICIEINNGELFFVASENSYNDDNINTPKEIDDTVTSNNNYENDLDQVILNFNSESAPGLSDEKLNLTIQNDAYIPKYDSNGTSYIEQHDVNELNVFFYLDAQKVPEGENNVNLTSSIDTALLEQPKIYTFFSSEFINNVNKTVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKTIIESKYNSYTLEEKNELTNKYNIEQIENELNQKVSIAMNNIEIFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 12 - protein, 1259 aa, synthetic construct
MPKINSFNYNDPVNDNTILYIKPGGCQQFYKSFNIMKNIWIIPERNVIGTIPQNFLPPTSLKNGDSSYYDPNYLQNDQEKDRFLKIVTKVFNRINDNLSGRILLEELSKANPYLGNDNTRDDDFIINDGSAVPIQFSNGSQSILLPTVIIMGAEPDLFETNSSNVSLINNYSPSNHGFGSIAIVTFSPEYSFRFNDNSMNEFIQDPALTLIHELIHSLHGLYGAKGITTKYTITQQQNPLITNIRGINIEEFLTFGGNNLNIITSSQLNDIYTNLLDDYKKIASKLSKVQVSNPQLNPYKDVFQEKYGLDKNASGIYSVNINKFNDIFKKLYSFTEFDLATKFQVKCRETYIGQYKYFKLSNLLNDSIYNISEGYNINTLNVNFRGQNPNLNPRIITPITDRGLVKKIIRFCKNIVSVKGIRKSICIEVNNGDLFFVASEKSYNNDSINIPKEIDDTVTLNNNYENDLDQVILNFNSESAPGLSDKKLNISIQDDVYIPKYDSNGTSDIEQYDVSELNVFFYLDAQKVPEGENNVNLTSSIDTALLEQSKIYTFFSSEFINNVNKPVQAALFVGWIQQVLVDFTTEATQKSTVDKIADISIVVPYIGLALNIGNESQKGNFKDALELLGAGILLEFVPELLIPTILVFTIKSFLGSSDNKNKVIKAINNALKERDEKWKEVYSFIVSNWITKINTQFNKRKEQMYQALQNQVNALKTIIESKYNSYTLEEKNELTNKYDIEQIENELNQKVSIAMNNIDRFLTESSISYLMKLINEVKINKLREYDENVKTYLLDYITKHGSILGESQQELNSMIIDTLNNSIPFKLSSYTDDKILISYFNKFFKRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 13 - protein, 1259 aa, synthetic construct
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGGILLEELSKANPYLGNDNTPNNQFHIGDASAVEIKFSNGSQSILLPTVIIMGAEPDLFETNSSNISLKNNYMPSNHGFGSIAIVTFSPEYSFRFNDNSMNEFIQDPALTLMHELIHSLHGLYGAKGITTKYTITQQQNPLITNIRGTNIEEFLTFGGTDLNIITNAQSNDIYTNLLADYKKIASKLSQVQVSNPQLNPYKDIFQEKYGLDKNASGIYSVNINKFDDIFKKLYSFTEFDLATKFQVKCRQTYIGQYKYFKLSNLLNNSIYNISEGYNINTLKVNFRGQNTNLNPRIITQLTGRGLVKKIIRFCKNIVFSKGITKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKTIIESKYNSYTLEEKNELTNKYNIEQIENELNQKVSIAMNNIEIFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 14 - protein, 1259 aa, synthetic construct
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGGILLEELSKANPYLGNDNTPNNQFHIGDASAVEIKFSNGSQSILLPTVIIMGAEPDLFETNSSNISLKNNYMPSNHGFGSIAIVTFSPEYSFRFNDNSMNEFIQDPALTLMHELIHSLHGLYGAKGITTKYTITQQQNPLITNIRGTNIEEFLTFGGTDLNIITNAQSNDIYTNLLDDYKKIASKLSQVQVSNPQLNPYKDVFQEKYGLDKDANGIYSVNINKFNDIFKKLYSFTEFDLATKFQVKCRKTYIGHHKYFRLSDLLNDSIYNISDGYNINTLKVNFRGQNTNLNTRIITPITGRGVVRKIIRFCTNIFSPKGIRKSICIEVNNGELFFVASENSYNDDNINTSKEIDDTVTSNNNYENDLDQVILNFNSESAPGLSDEKLNLTIQDDAYIPKYDSNGTSDIEQYDVSELNVFFYLDAQKVPEGENNVDFTSSIDTALLEQPKIYTFFSSKFISNLNKTMQAALFVSWIQQVLVDFTTEATQKSTVDKIADISIVVPYIGLALNIGNEAQKGNFKDALELLGAGILLEFEPELLIPIILVFTIKSFLGSSDNKNKVIKAINNALKERDENWKEVYSFIVSNWMTKINTQFNKRKEQMYQALQNQVNAIKTIIESKYNSYTLEEKNELKNKYDIEQIENELNQTVSIAMNNIEIFLTESSISYLMKLINEVKINKLKEYDENVKTYLLDYIIKHGSILGESQQELNSMVIDTLNNSIPFKLSSYTDDKILISYFNKFFKRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 15 - protein, 1259 aa, synthetic construct
MPKINSFNYNDPVNDRTILYIKPGGCQQFYKSFNIMKNIWIIPERNVIGTIPQDFQPPTSLKNGDSSYYDPNYLQSNEEKDRFLKIVTKIFNRINDNLSGGILLEELSKANPYLGNDNTPDGDFIINDASAVPIQFSNGSQSILLPNVIIMGAEPDLFETNSSNISLINNYRPSNHGFGSIAIVTFSPEYSFRFNDNSMNEFIQDPALTLMHELIHSLHGLYGAKGITTKYTITQQQNSLITNIRGINIEEFLTFGGNDLNIITSSQFNDIYTNLLDDYKKIASKLSQVRVSNPQLNPYKDVFQEKYGLDKDASGIYSVNINKFNDIFKKLYSFTEFDLATKFQVKCRETYIGQYKYFQLSNLLNDSIYNISEGYNINNLKVNFRGQNANLNPRIITPITGRGLVKKIIRFCKNIVSVKGIRKSICIEVNNGELFFVASENSYNDDNINTPKEIDDTVTSNNNYENDLDQVILNFNSESAPGLSDEKLNLTIQDDAYIPKYDSNGTSDIEQHDVNELNVFFYLDAQKVPEGENNVNLTSSIDTALLEQPKIYTFFSSEFINNVNKPVQAVLFVSWIQQVLVDFTTEATQKSTVDKIADISIVVPYIGLALNIGNEAQKGNFKDALELLGAGILLEFVPELLIPTILVFTIKSFLGSSDNKNKIIKAINNALKERDEKWKEVYSFIVSNWITKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSTLGESQQELNSMVINTLNNSIPFKLSSYTDDKILISYFNKFFKRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 16 - protein, 1285 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 17 - protein, 1272 aa, synthetic construct
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 18 - protein, 1280 aa, synthetic construct
MPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 19 - protein, 1280 aa, synthetic construct
MPITINNFNYSDPVDNKNILYLDTHLNTLANEPEKAFRIIGNIWVIPDRFSRDSNPNLNKPPRVTSPKSGYYDPNYLSTDSEKDTFLKEIIKLFKRINSREIGEELIYRLATDIPFPGNNNTPINTFDFDVDFNSVDVKTRQGNNWVKTGSINPSVIITGPRENIIDPETSTFKLTNNTFAAQEGFGALSIISISPRFMLTYSNATNNVGEGRFSKSEFCMDPILILMHELNHAMHNLYGIAIPNDQRISSVTSNIFYSQYKVKLEYAEIYAFGGPTIDLIPKSARKYFEEKALDYYRSIAKRLNSITTANPSSFNKYIGEYKQKLIRKYRFVVESSGEVAVDRNKFAELYKELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDIKTDIFLSKDINEETEVIDYPDNVSVDQVILSKNTSEHGQLDLLYPIIEGESQVLPGENQVFYDNRTQNVDYLNSYYYLESQKLSDNVEDFTFTTSIEEALDNSGKVYTYFPKLADKVNTGVQGGLFLMWANDVVEDFTTNILRKDTLDKISDVSAIIPYIGPALNISNSVRRGNFTEAFAVTGVTILLEAFQEFTIPALGAFVIYSKVQERNEIIKTIDNCLEQRIKRWKDSYEWMIGTWLSRITTQFNNISYQMYDSLNYQADAIKDKIDLEYKKYSGSDKENIKSQVENLKNSLDIKISEAMNNINKFIRECSVTYLFKNMLPKVIDELNKFDLKTKTELINLIDSHNIILVGEVDRLKAKVNESFENTIPFNIFSYTNNSLLKDIINEYFNRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 20 - protein, 1276 aa, synthetic construct
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 21 - protein, 1278 aa, synthetic construct
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 22 - protein, 1277 aa, synthetic construct
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 23 - protein, 1273 aa, synthetic construct
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHELIHVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSNTKNSLCITVNNRDLFFIASQESYGENTINTYKEIDDTTTLDPSFEDILDKVILNFNEQVIPQMPNRNVSTDIQKDNYIPKYDYNRTDIIDSYEVGRNYNTFFYLNAQKFSPNESNITLTSSFDTGLLEGSKVYTFFSSDFINNINKPVQALLFIEWVKQVIRDFTTEATKTSTVDKLKDISLVVPYIGLALNIGDEIYKQHFAEAVELVGAGLLLEFSPEFLIPTLLIFTIKGYLTGSIRDKDKIIKTLDNALNVRDQKWKELYRWVVSKWLTTINTQFNKRKEQMYKALKNQATAIKKIIENKYNNYTTDEKSKIDSSYNINEIERTLNEKINLAMKNIEQFITESSIAYLINIINNETIQKLKSYDDLVRRYLLGYIRNHSSILGNSVEELNSKVNNHLDNGIPFELSSYTNDSLLIRYFNKNYGRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 24 - protein, 1322 aa, synthetic construct
MDVLEMFDVNYESPILESFDSTTQSLNDVHVFMSRIQMSAYDADGEGRIEYRNLKLYEISSGIFISTDRLDTGASGVEDDHEMVDYYSSARLTREFLGESLDSQKSDYFEGIKKVFSFYKNKCNESRYIKEFFEEIQFRNICGFPKQAGTSSTDIFDQFNSVDVLLQDPVTSVWNKKVGSKKANIVIIPPATNLPITEACATAGFQPEGFPKLGSGSFFTVQFDPFFSTRFKAHETDDVALLDPTLTLLHEMTHGLHFQKGIANPVNRSGETPAWATTWGRVTGDNDAFKETPMEELLTFNKHTIDDDIEISDHLKSTYIGFLYNGRNEDDPTESVDGVYQNVSSFLNQYRGFEISSDFQHFIESCYGVKYNQESKKFIVNPRNIKRYVQDGFFIDEAKFARILNIKTRSYYTLMPDNLGVWSYRVDILNRLRETFDEDRGLLSQELDFHTALTPVVSENPALELEVAGMQRMVSLPKIKASYLPSDIKIKNFTGQKISHDTILDTNISGIIISKIKYKSDFVVDESMPRSSLNTTNYNLSPIKGTKFETDIRDKTSVKVTVSEITAPMINHVMKLDNSKVLTERPSLNEDLEETFKNTKDVYIPKTTAMMKLKEGADQTLGAVGFAVWSGQILEDLYNLAQKKEVSIDQIKDDLMSILPFYCAYKNLSAEKYEQAFANATLDAFLIFATDGGGFAGLGITVGAIAINSMYAKAETMEAYDSMFGKYVDQYQNDIKNFTLNAYVQWENNILSRLWNESRLAITGFRNMLKTVKTVMEFDATNQAYSEEDRKIIKAKCEEIFSEFPMLMQTFAKNSMTANLENASKIFNDIVWQKIKEELDQYVIDSKKYFLDSLEEAYNNGSISAESYYKYQTEAREKFVSPREVIDLYIAAHDTVVKRKRYIRRYSRRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

SEQ ID NO: 25 - protein, 1304 aa, synthetic construct
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNRIKSSSVLNMRYKNDKYVDTSGYDSNINIGSKVNKYPTNKNQFQLFNLESSEVNISQNDYIIYDNKYKNFSISFWVRIPKYFNSISLNNEYTIINCMRDNNSGWKVSLNYGEIIWTLQDNAGINQKLAFNYSQMINISDYINKWIFVTITNDRLGDSKLYINGNLIDQKSILNLGNIHVSDNILFKIVNCRDTHRYIGIRYFNIFDKELDETEIQTLYSNEPNTNILKDFWGNYLLYDKEYYLLNVLKPNNYIDGRKDSTLSIKGPRGSVMTTNIYLNSSLYRGIKVKIQRVNNSSTNDNLVRKNDQVYINFVASKTGLGPLYADTATTNKEKTIKISSSGNRFNQVVVMNSVGNNCTMNFKNNNGNNIGLLGFKADTVVASTWYYTHMRDHTNSNGCFWNFISEDDGWGERPL

(19) United States
(12) Patent Application Publication
(10) Pub. No.: US 2026/0275322 A1
(43) Pub. Date: Sep. 17, 2026

(54) ENGINEERED TYPE E BOTULINUM NEUROTOXIN FOR THERAPEUTIC AND COSMETIC APPLICATIONS

(71) Applicant: The Regents of the University of California, Oakland, CA (US)

(72) Inventors: Rongsheng Jin, Irvine, CA (US); Zheng Liu, Irvine, CA (US); Linfeng Gao, Irvine, CA (US)

(21) Appl. No.: 19/469,674

(22) PCT Filed: Mar. 27, 2024
(86) PCT No.: PCT/US24/21782

§ 371 Date: Sep. 26, 2025

Related U.S. Application Data
(60) Provisional application No. 63/492,449, filed on Mar. 27, 2023.

(51) Int. Cl. C12N 9/52

(52) U.S. Cl. CPC C12N 9/52; C12Y 304/24069

(57) ABSTRACT

Serotype E botulinum neurotoxin (BoNT/E) is one of the major causes of human botulism and paradoxically also a promising therapeutic agent. However, the molecular mechanism by which BoNT/E recognizes synaptic vesicle glycoprotein 2A (SV2.A) and 2B (SV2B), but no closely related SV2C, as its neuronal receptors has been challenging to resolve. The Inventor, surprisingly, was able to obtain crystal structures of the receptor-binding domain of BoNT/E (H_{C}E) in complex with SV2.A. These structures reveal that H_{C}E engages both specific peptide fragments and an N-glycan of SV2A, as well as SV2B in a similar manner. Structure-based mutagenesis and functional studies demonstrate that both the peptide- and glycan-based associations are essential for SV2A/2B-mediated cell entry of BoNT/E and for its potent neurotoxicity. Thus, the engineering of BoNT/E variants for new clinical applications is described herein.

### CROSS-REFERENCE TO RELATED APPLICATIONS

This application claims benefit of U.S. Provisional Application No. 63/492,449 filed Mar. 27, 2023, the specification of which is incorporated herein in their entirety by reference.

### STATEMENT REGARDING FEDERALLY SPONSORED RESEARCH OR DEVELOPMENT

This invention was made with government support under Grant Nos. R21AI123920, R21AI156092, R01AI158503, R01AI125704, R01AI139087, R01NS080833, R01AI132387, R21AI163178, P30 GM124165, and S10OD021527 awarded by National Institute of Health. The government has certain rights in the invention.

### REFERENCE TO AN ELECTRONIC SEQUENCE LISTING

The contents of the electronic sequence listing (UCI 23_04 PCT.xml; Size: 51,767 bytes; and Date of Creation: Mar. 26, 2024) is herein incorporated by reference in its entirety.

### FIELD OF THE INVENTION

Disclosed herein are enhanced variants of serotype E botulinum neurotoxins (BoNT/E) comprising a plurality of mutations designed to augment BoNT/E potency and finely tune receptor binding specificity. The present invention also encompasses formulations containing these optimized neurotoxins, along with methods for their application.

### BACKGROUND OF THE INVENTION

Botulinum neurotoxins (BoNTs) are the causative agents of the neuroparalytic disease botulism. There are seven major BoNT serotypes (termed BoNT/A-G) including more than 40 subtypes. New BoNT serotypes have been identified recently (e.g., BoNT/HA and BoNT/X) as well as several BoNT-like genes in non-clostridial species such as Enterococcus faecium (BoNT/En) and Weissella oryzae (BoNT/Wo). Among these BoNTs BoNT/A and BoNT/B are approved by the FDA for clinical and aesthetic indications. The extraordinary potency of BoNTS relies on highly specific recognition and uptake by motor neurons. A widely accepted dual-receptor model suggests that the receptor-binding domain (H_{C}) of BoNTs synergistically binds complex gangliosides and specific protein receptors on the neuron surface. Complex gangliosides are present abundantly on nerve cells and serve to enrich toxins, and most BoNTs possess a highly conserved ganglioside-binding “SxWY” motif. However, BoNTs have developed diverse binding strategies for their corresponding protein receptors. For example, BoNT/A and BoNT/B exploit distinct protein receptors, which may contribute to their differences in pharmacological and clinical profiles. BoNT/E has recently emerged as a promising new drug candidate due to its faster onset of action and shorter duration of effect when compared to BoNT/A and BoNT/B. Thus, the present invention features formulations and methodologies incorporating BoNT/E with multiple mutations aimed at enhancing BoNT/E potency and precisely refining receptor binding specificity. Furthermore, the invention serves to expedite therapeutic advancements and the engineering of BoNT/E for innovative neurotoxin products, while also providing insights to guide the development of novel strategies for BoNT inhibitors.

### BRIEF SUMMARY OF THE INVENTION

It is an objective of the present invention to provide compositions that allow for increases in the potency and modulating receptor binding specificity of BoNT/E as well as methods of use, as specified in the independent claims. Embodiments of the invention are given in the dependent claims. Embodiments of the present invention can be freely combined with each other if they are not mutually exclusive.

The present invention features novel receptor-binding domain (H_{C}) of BoNT/E (H_{C}E) variants characterized by enhanced affinities for their human receptors, synaptic vesicle glycoprotein 2 (SV2), which has three homologous isoforms, SV2A, 2B, and 2C. Additionally, through engineering efforts, the modified H_{C}E demonstrates the capability to effectively recognize SV2C—a feat unattainable by the wild-type H_{C}E. This successful retargeting of BoNT/E to SV2C allows for expanded cosmetic and therapeutic applications.

In some embodiments, the present invention features a modified serotype E botulinum neurotoxin (BoNT/E) comprising a plurality of mutations, wherein one or more mutations decrease endogenous BoNT/E receptor binding function; and wherein one or more mutations mimic endogenous botulinum neurotoxin A (BoNT/A) receptor binding. In other embodiments, the present invention features a modified serotype E botulinum neurotoxin (BoNT/E) comprising a sequence according to SEQ ID NO: 2 or SEQ ID NO: 3.

As described herein, key amino acids were identified on BoNT/E that are responsible for binding the protein-moiety and glycan-moiety of SV2A and SV2B, and amino acids that cause BoNT/E not to recognize SV2C. Based on the key amino acids identified, the receptor-binding domain of BoNT/E (H_{C}E) was modified in order to enhance its binding to SV2. Thus, one of the unique and inventive technical features of the present invention is a modified BoNT/E. Without wishing to limit the invention to any theory or mechanism, it is believed that the technical feature of the present invention advantageously provides for enhanced binding to SV2. The general principle of the engineering is to introduce a BoNT/A-like SV2 binding mode to BoNT/E, while removing the endogenous BoNT/E-like SV2 binding. The improved HE could have at least two advantages: (1) it will adopt a BoNT/A-like binding mode that has higher affinity for SV2 than the endogenous BoNT/E-like binding mode, and (2) the engineered H_{C}E will recognize all three SV2 isoforms, while the wild-type H_{C}E doesn't recognize SV2C, which will broaden receptor coverage for BoNT/E. None of the presently known prior references or works have the unique inventive technical feature of the present invention.

Furthermore, the inventive technical features of the present invention contributed to a surprising result. For example, the neurotoxicity of BoNT/A-G and the administered dose of a BoNT product in clinic are largely determined by the affinity of its He domain to the human receptor. Contrastingly, BoNT/E exhibits relatively lower neurotoxicity compared to BoNT/A, necessitating higher protein doses for efficacy. However, elevated doses can precipitate adverse effects associated with unintended toxin dispersion from the injection site, alongside heightened risks of developing neutralizing antibodies, compromising the effectiveness of subsequent treatments. Unexpectedly, the Inventors identified specific mutations within BoNT/E that enhance its potency and specificity, thereby minimizing the required dosage across diverse cosmetic and clinical applications.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, 1C, 1D, 1E, 1F, and 1G shows the structure of the H_{C}E-G6 complex and the designs of SV2A-G6 fusion protein. FIG. 1A shows H_{C}E pre-incubated with or without a nanobody (aka, VHH) G6 was incubated with liposomes containing 70/20/10 mol % BrPC/DOPS/GT1b. After liposomes were pelleted, HE and G6 in the input (I), supernatant(S), and pellet (P) fractions were analyzed by SDS-PAGE and Coomassie blue staining (a representative result is shown, n=3). FIG. 1B shows a cartoon representation of the H_{C}E-G6 complex with H_{C}E and G6. The N- and C-terminal subdomains of H_{C}E are referred to as H_{CN}E and H_{CC}E, respectively. FIG. 1C shows a close-up view of the interface between H_{C}E and G6 highlighted in the box in (FIG. 1B). Key interacting residues are shown as sticks. FIG. 1D shows a G6 occupies the ganglioside-binding pocket on H_{C}E. The G6-bound H_{C}E (top cartoon) is superimposed with the GD1a-bound H_{C}E (bottom cartoon) (PDB: 70VW). G6 and GD1a are shown as a surface model and a stick model, respectively, and two crucial ganglioside-binding residues W1224 and Y1225 are shown as sticks. FIG. 1E shows a schematic diagram showing the design of a SV2A-G6 fusion protein. FIG. 1F shows the G6^{AA} mutant (D100A/D115A) showed no detectable binding to H_{C}E in a pull-down assay with H_{C}E as a prey and the His/SUMO-tagged G6^{WT} or G6^{AA} as a bait. FIG. 1G shows that HE recognizes SV2A in a pH-dependent manner. Biotin-labeled SV2A-G6^{AA} as bait could pull down H_{C}E at pH 7.5, but not pH 4.6 or 5.0. Representative results are shown in panels FIG. 1A, FIG. 1F, and FIG. 1G (n=3).

FIGS. 2A, 2B, 2C, 2D, and 2E shows an engineered SV2A-SV2C chimera maintains a SV2A-like binding to BoNT/E. FIG. 2A shows a schematic diagram showing the designs of SV2A-SV2C chimeras where the sequences of SV2C-L4 and SV2A-L4 are shown as solid and hollow bars, respectively. The structure of SV2C-L4 is shown as a cartoon with residues K518, 1538, N559, and the N559 glycan shown as sticks (PDB: 5JLV). FIG. 2B shows the chimeric SV2Ac-G6^{AA} fusion protein is mono-dispersed in solution based on a gel-filtration analysis. FIG. 2C shows a pull-down assay was performed using H_{C}E as a prey and the His-tagged SV2-L4 or SV2-G6^{AA} fusion proteins as baits. Only SV2A-G6^{AA} and SV2Ac-G6^{AA} were able to pull down HE (indicated by asterisks), but not other SV2-G6^{AA} fusion proteins or the stand alone SV2-L4 variants. A representative result is shown (n=3). FIG. 2D and FIG. 2E shows the full-length SV2A and SV2Ac were expressed via lentiviral transduction in mouse cortical neurons cultured from SV2A/B double knockout (KO) mice. Neurons were exposed to H_{C}E (5 min) (FIG. 2D) or BoNT/E at the indicated concentration (24 hours in medium) (FIG. 2E). Cell lysates were harvested and analyzed by immunoblot assays. The synaptic vesicle protein, Synaptotagmin 1 (Syt-1), was detected as a loading control. The SNAP-25 antibody can detect both the full-length SNAP-25 and the fragment generated after cleavage by BoNT/E (marked with *). SV2A(+/+)/SV2B(−/−) neurons were analyzed in parallel as a positive control. A representative result is shown (n=2).

FIGS. 3A, 3B, 3C, and 3D shows the overall structure of the H_{C}E-SV2Ac-G6 complex. FIG. 3A shows a cartoon representation of the H_{C}E-SV2Ac-G6 complex (H_{C}E, lemon; SV2Ac, orange; G6, pink). H_{C}E engages the protein moiety of SV2Ac mainly through two loops on its H_{CC}E domain (teal and purple boxed areas). The dotted line indicates the flexible linker between SV2Ac and G6 that was invisible in this structure. FIG. 3B shows the H_{C}E-SV2Ac interface viewed from a different angle. FIG. 3C and FIG. 3D shows a close-up view of the interfaces between H_{C}E and SV2Ac that are highlighted boxes in FIG. 3A and FIG. 3B with key interacting residues shown as sticks.

FIGS. 4A, 4B, 4C, 4D, and 4E shows the structure of the sialic acid-bound H_{C}E-SV2Ac complex and a comparison of the glycan-binding modes between BoNT/E and BoNT/A. FIG. 4A shows a cartoon representation of the sialic acid (SIA)-bound H_{C}E-SV2Ac complex with H_{C}E colored in lemon, SV2Ac in orange, and sialic acid in red. An omit electron density map for sialic acid contoured at 1.5 σ was overlaid with the final refined model. FIG. 4B shows a close-up view of the interactions between H_{C}E and sialic acid. One water molecule (W) that mediates the H_{C}E-SIA binding is shown as a cyan sphere. FIG. 4C shows the H_{C}E-SV2Ac-SIA complex and the H_{C}A-SV2C complex (PDB: 5JLV) were superimposed based on HE and H_{C}A. The SIA (sialic acid), NAG (N-acetylglucosamine), and the SV2C glycan are shown as sticks. The SIA-binding and the SV2C-glycan-binding pockets are highlighted in red and blue boxes, respectively. FIG. 4D shows a close-up view into the SV2C-glycan-binding pocket, revealing the differences between H_{C}A and H_{C}E in this area. H_{C}E residue Q1250 would clash with the SV2C glycan based on the superposition. FIG. 4E shows a close-up view into the SIA-binding pocket shows that this pocket is partly conserved between H_{C}E and H_{C}A.

FIGS. 5A and 5B shows simultaneous binding to the protein- and glycan-moiety of SV2A is crucial for neuron binding and neurotoxicity of BoNT/E. FIG. 5A shows rat cortical neurons were exposed to a high K⁺ buffer containing 200 nM H_{C}E for 5 min at 37° C. Cells were washed three times, and binding of the biotinylated H_{C}E variants was detected by immunoblot analysis of cell lysates using Streptavidin-HRP. SNAP-25 was detected as a loading control.

FIG. 5B shows MPN assay showed drastically decreased neurotoxicity of BoNT/E when its protein-protein and protein-sialic acid binding interfaces, respectively, were disrupted by mutagenesis. Graph shows means #s.d. of n=3 biologically independent experiments for triple mutant R1100G/H1158G/F1160G and n=5 for all other mutants.

FIGS. 6A, 6B, and 6C shows BoNT/E differentiates SV2A and SV2B from SV2C. FIG. 6A shows amino acid sequence alignment among human SV2A, SV2B, and SV2C in the L4 region (prepared using MultAlin and ESPript 3.0). Identical residues are indicated with white letters on a red background; conserved residues are in red letters, varied residues are in black letters. The SV2A residues that are recognized by H_{C}E are indicated by orange triangles. Residue numbers of SV2A and the secondary structures of chimeric SV2Ac are shown on the top. FIG. 6B shows the WT SV2A or SV2A^{Y535T/537E} mutant was expressed in SV2A/B KO mouse cortical neurons via lentiviral transduction. Neurons were exposed to the indicated toxins (14 hours in medium). Cell lysates were harvested and analyzed by immunoblot assays. Actin served as a loading control. Left panel: representative immunoblots. Right panel: the percentage of SNAP-25 cleavage by BoNT/E in the left panel was quantified using ImageJ by comparing the amount of cleavage products versus the intact SNAP-25. Error bar represents SD from three independent experiments. FIG. 6C shows the WT SV2C or SV2C^{T521Y/E543Y} mutant was expressed in SV2A/B KO neurons. Neurons were exposed to the indicated concentrations of BoNT/E (24 hours in the medium). Cell lysates were harvested and analyzed by immunoblot assays. A representative result is shown (n=2). The cleaved SNAP-25 is marked with *.

FIG. 7 shows proposed models for simultaneous binding of BoNT/E and BoNT/A to the membrane-anchored SV2 and gangliosides. FIG. 7A shows proposed binding modes of BoNT/E (PDB: 3FFZ) and BoNT/A (PDB: 3BTA) with the membrane-bound SV2 and gangliosides. The holotoxins are positioned based on the structures of the H_{C}E-SV2Ac and the HA-SV2C (PDB: 5JLV) complexes. A representative complex-type N-glycan (sticks, PDB: 3QUM) is modeled to represent the N-glycan of SV2A. The gangliosides GD1a and GT1b are modeled based on the structures of HE-GD1a (PDB: 70VW) and HA-GT1b (PDB: 2VU9.

FIG. 7B shows the structures of the H_{C}E-SV2Ac and the H_{C}A-SV2C (PDB: 5JLV) complexes with the modeled gangliosides are superimposed based on H_{C}E and H_{C}A. The view angle on the left panel is identical to that shown in panel (FIG. 7A). A different view with a rotation ~115° about a vertical axis is shown on the right panel. Two equivalent β-sheets (88) on SV2Ac-L4 and SV2C-L4 are highlighted as a marker to show the similar orientations of SV2Ac and SV2C relative to the membrane in this putative model. FIG. 7C shows a surface representation of BoNT/E holotoxin in complex with SV2A and GD1a. BoNT/E adopts a “closed-wing” conformation, in which H_{C}E and LC/E are located on the same side of the long helical H_{N}E. FIG. 7D shows surface representation of BoNT/A holotoxin in complex with SV2C and GT1b. BoNT/A has an “open-wing” conformation, in which H_{C}A and LC/A are located on the opposite sides of the long helical H_{N}A.

FIGS. 8A, 8B, 8C, and 8D show the receptor-binding modes compared among H_{C}A, H_{C}B, and H_{C}E. FIG. 8A shows amino acid sequence alignment between H_{C}A and H_{C}E focusing on the major area where H_{C}A recognizes the protein moiety of SV2C. Identical residues are indicated with white letters on a red background and similar conserved residues are in red letters. The residues of HA that bind to SV2C protein moiety are indicated by yellow triangles, and the major differences between HA and H_{C}E are highlighted in a red box. Residue numbers and the secondary structures of H_{C}E are shown on the top. FIG. 8B shows G6 occupies the ganglioside-binding pocket on H_{C}E based on comparison of the H_{C}E-G6 complex and the H_{C}A-GT1b complex (PDB: 2VU9): H_{C}A (cartoon); GT1b (sticks); H_{C}E cartoon); G6 (surface and cartoon). Residues “WY” of the highly conserved “SxWY” motif are shown as sticks. FIG. 8C shows a model of H_{C}A in complex with SV2C and GT1b based on the structures of the H_{C}A-SV2C (PDB: 5JLV) and the H_{C}A-GT1b (PDB: 2VU9) complexes: H_{C}A; SV2C; GT1b (sticks). FIG. 8D shows H_{C}B in complex with Syt-II and GD1a (sticks) (PDB: 4KBB).

FIG. 9 shows SV2Ac mediates BoNT/A entry into cultured cortical neurons. The full-length SV2A and SV2Ac were expressed via lentiviral transduction in cortical neurons cultured from SV2A/B double KO mice. Neurons were exposed to BoNT/A (100 μM, 24-hour incubation in medium). Cell lysates were collected and analyzed by immunoblot. The SNAP-25 antibody can detect both the intact SNAP-25 and the smaller fragment of SNAP-25 after cleavage by BoNT/A (marked with *). Syt-1 was detected as a loading control. SV2A(+/+)/SV2B(−/−) neurons were tested in parallel as a positive control. A representative result is shown (n=2).

FIGS. 10A, 10B, 10C, 10D, 10E, and 10F shows the structure of the H_{C}E-SV2Ac-G6 complex and mutagenesis studies on H_{C}E. FIG. 10A shows the structure of the H_{C}E-SV2Ac-G6 complex (H_{C}E, lemon; G6, pink; SV2Ac, orange) is superimposed with the H_{C}E-G6 complex (grey) based on H_{C}E. It shows that the SV2Ac-linked G6 and the stand alone G6 bind to H_{C}E in an identical manner. FIG. 10B shows two different interfaces were observed between SV2Ac and H_{C}E in the crystal of the H_{C}E-SV2Ac-G6 complex (11 and 12). FIGS. 10C and 10D shows the interacting residues of SV2Ac and H_{C}E at 11 and 12 are shown as sticks.

FIG. 10E shows the characterization of the interactions between H_{C}E and SV2A by structure-based mutagenesis. Pull-down assays were performed using H_{C}E variants as preys and the His-tagged SV2A-G6^{AA} as a bait. FIG. 10F shows SV2A^{D514G} (D514 of SV2A was replaced with the equivalent G500 of SV2C) and SV2ANT bind to H_{C}E with similar affinities based on a pull-down assay with H_{C}E as a prey and the equal amount of His-tagged SV2A^{D514G}-G6^{AA} and SV2A^{WT}-G6^{AA} fusion proteins as baits. Representative results are shown in panels FIGS. 10E and 10F (n=3).

FIGS. 11A, 11B, and 11C shows BoNT/E grips the distant tip of SV2A glycan, FIG. 11A shows the electron density of N-acetylglucosamines (NAG) of the SV2Ac-N573 glycan observed in the H_{C}E-SV2Ac-G6 complex. An omit electron density map contoured at 1.5 σ was overlaid with the final refined model. FIG. 11B shows the electron densities for an unknown molecule were observed in a pocket located at the N-terminal sub-domain of H_{C}E (H_{CN}E). An omit electron density map contoured at 1.5 σ and a 2F_{O}-F_{C} electron density map for five selected residues (sticks) in this area contoured at 1 σ were overlaid with the final refined model. FIG. 11C shows structure modeling of H_{C}E binding to the SV2A N573 glycan. Two representative complex-type N-glycans are modeled to represent the SV2A-N573 glycan based on the structures of a glycan from human prostate-specific antigen (upper panel, red sticks, PDB: 3QUM) and a sialylated human IgG Fc (lower panel, pale cyan sticks, PDB: 4BYH). The terminal sialic acid of both glycans could reach the sialic acid-binding pocket on HE (orange dotted area). A schematic representation of a complex-type N-glycan structure is shown at the bottom.

FIG. 12 shows H_{C}E, SV2A-G6^{AA} and SV2C-G6^{AA} variants adopt wild-type-like structures. The thermal stability of proteins was measured using a fluorescence-based thermal shift assay on a StepOne real-time PCR system (ThermoFisher). Protein melting was monitored using a hydrophobic dye, SYPRO Orange (Sigma-Aldrich), as the temperature was increased in a linear ramp from 25° C. to 95° C. The midpoint of the protein-melting curve (Tm) was determined using the software provided by the instrument manufacturer. The data are presented as means #s.d. (n=3). All the H_{C}E, SV2A-G6MA and SV2C-G6^{AA} variants showed Tm values comparable to the wild-type proteins, indicating correct protein folding.

FIG. 13 shows a dose-response curve of the wild-type BoNT/E1 in the mouse phrenic nerve hemidiaphragm assay. To allow comparison of the altered neurotoxicity of mutants with BoNT/E1 wild-type, BoNT/E1 wild-type was tested in three concentrations (2.0 μM, n=6; 4.0 μM, n=8; 8.0 μM, n=5 biologically independent experiments). A logarithmic function (y (BoNT/E1 wt; 2.0, 4.0, 8.0 μM)=−23.61 In(x)+104.11, R2=0.999) was fitted to the data points (mean #SD) and used to convert paralytic-halftimes of BoNT/E1 mutants into concentrations of the wild-type and finally expressed as relative neurotoxicity.

FIGS. 14A and 14B shows Bio-layer interferometry (BLI) analyses of SV2A-G6^{AA} and SV2C-G6^{AA} variants binding to H_{C}E. FIG. 14A shows equal amounts of biotinylated SV2A-G6^{AA} or SV2A^{Y535T/Y657D}-G6^{AA} (400 nM) were immobilized on streptavidin (SA) biosensors as ligands and 1 μM of HE was used as the analyte. FIG. 14B shows equal amounts of biotinylated SV2C-G6^{AA} or SV2C^{T621Y/D543Y}-G6^{AA} were immobilized on streptavidin (SA) biosensors as ligands and 1 μM of HE was used as the analyte.

FIG. 15 shows SV2C (T521Y/E543Y) mediates BoNT/A entry into cultured neurons. The full-length SV2C and SV2C (T521Y/E543Y) were expressed via lentiviral transduction in cortical neurons cultured from SV2A/B KO mice. Neurons were exposed to BoNT/A (1 nM, 12 hours in medium). Cell lysates were harvested and analyzed by immunoblot assays. Cleaved SNAP-25 is marked with *. Expression of SV2C is detected using a polyclonal anti-SV2C antibody. Both SV2C WT and SV2C (T521Y/E543Y) mediated entry of BoNT/A, resulting in cleavage of SNAP-25. A representative result is shown (n=2).

FIG. 16 shows Sequence alignment among twelve BoNT/E subtypes. The amino acid sequences of BoNT/E1-E12 are taken from GenBank: AFV91350 (E1), EF028404 (E2), ABM73980 (E3), EEP52948 (E4), AB037704 (E5), ABY878 (E6), AER11391 (E7), AER11392 (E8), AFV91339 (E9), AII82300 (E10), KF861879 (E11), and KF929215 (E12). Key H_{C}E residues that are recognized by VHH G6 are Indicated by green triangles. H_{C}E residues that interact with SV2Ac peptide or sialic acid are labeled by orange ovals or blue stars, respectively.

FIGS. 17A, 17B, 17C, and 17D shows the structure-based design of H_{C}E (V3.5). FIG. 17A shows the crystal structure of the wild-type H_{C}E in complex with its receptor SV2 (SV2Ac). The structure of H_{C}E is shown as green ribbons and a transparent surface presentation, while SV2Ac is shown as a blue ribbon presentation. The red circle highlights the area that we performed mutagenesis to create a new binding site for SV2 with a higher affinity. FIG. 17B shows the crystal structure of H_{C}E (V3.5) in complex with SV2Ac. FIG. 17C shows a surface presentation of H_{C}E highlighting the mutated amino acids in V3.5. Residues colored in blue are the ones to improve protein-mediated binding; residues colored in green are the ones to improve glycan-mediated binding; and residues colored in cyan are for both protein- and glycan-mediated binding. The three residues colored in red are the ones to disrupt the endogenous SV2-binding site on the WT H_{C}E, so H_{C}E (V3.5) has to use the newly engineered binding site for SV2. FIG. 17D shows the structure of H_{C}E (V3.5) in complex with SV2Ac with the engineered areas highlighted as in (FIG. 17C).

FIGS. 18A and 18B shows H_{C}E-V1 has a gain-of-function to bind SV2C and also has an enhanced binding to SV2A. FIG. 18A shows a pull-down performed using the biotin-labeled SV2C, SV2Ac, and SV2A-G6 (G6^{AA}). SV2Ac is a chimera of SV2A and SV2C, which has a better biochemical behavior compared to the WT SV2A while functionally mimicking SV2A. SV2A-G6 is a fusion protein composed of the WT SV2A and a nanobody G6 (G6^{AA} with D100A/D115A) that weakly binds to H_{C}E and therefore enhances SV2A binding to H_{C}E. All three proteins appear as a range of smear bands because they are glycosylated. Sample #1, HA; #2, H_{C}E-WT; #3, H_{C}E-V1; #4, H_{C}E-V1-2G; #5, H_{C}E-V1-3G. FIG. 18B shows the protein inputs showing equal amounts of H_{C}A or H_{C}E variants were used in this assay. H_{C}E-V1-2G (#4) carries H1158G/F1160G mutations, while HE-V1-3G (#5) carries R1100G/H1158G/F1160G mutations. These mutations are introduced to disrupt the endogenous SV2A-binding site on H_{C}E, but they did not affect H_{C}E-V1 binding to SV2Ac or SV2A-G6. These results thus demonstrate that H_{C}E-V1 in fact uses the engineered HA-binding interface to bind SV2Ac and SV2A-G6.

FIG. 19 shows H_{C}E-V3.5 has a gain-of-function to bind SV2C. In this assay, a biotin-labeled SV2C strongly pull down HA-WT and H_{C}E-V3.5, but not H_{C}E-WT.

FIG. 20 shows SDS-PAGE analysis of the recombinantly expressed and purified BoNT/E-V3.5. The left lane is the reducing condition where the upper band is the H_{N}-H_{C} fragment and the lower band is the LC. The right lane is the non-reducing condition, where BoNT/E-V3.5 runs as a single band since the H_{N}-H_{C} domain and the LC are linked by a disulfide bond. This pattern of BoNT/E-V3.5 is identical to the wild-type BoNT/E.

FIG. 21 shows the determination of biological activity of BoNT/E variants by MPN hemidiaphragm assay. BoNT/E-V3.5 was measured in three protein concentrations of 2.5, 5.0 and 10.0 μM in 4-5 biological replicates. Above diagram shows mean #SD. Comparison of BoNT/E-V3.5 with BoNT/E1 wild-type reveals an increase of 2.15-fold in biological activity and with BoNT/E3 wild-type an increase of 1.65-fold in biological activity.

### TERMS

Disclosed are various peptides, solvents, solutions, carriers, and/or components to be used to prepare compositions to be used within the methods disclosed herein. Also disclosed are the various steps, elements, amounts, routes of administration, symptoms, and/or treatments that are used or observed when performing the disclosed methods, as well as the methods themselves. These and other materials, steps, and/or elements are disclosed herein, and it is understood that when combinations, subsets, interactions, groups, etc. of these materials are disclosed, that while specific reference of each various individual and collective combination and permutation of these compounds may not be explicitly disclosed, each is specifically contemplated and described herein. It is also to be understood that the terminology used herein is for the purpose of describing particular embodiments only and is not intended to be limiting.

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

The terms “polypeptide” and “protein” are used interchangeably to refer to a polymer of amino acid residues, comprising natural or non-natural amino acid residues, and are not limited to a minimum length. Thus, peptides, oligopeptides, dimers, multimers, and the like are included within the definition. Both full-length proteins and fragments thereof are encompassed by the definition. The terms also include post-translational modifications of the polypeptide, including, for example, glycosylation, sialylation, acetylation, and phosphorylation. Furthermore, a “polypeptide” herein also refers to a modified protein such as single or multiple amino acid residue deletions, additions, and substitutions to the native sequence, as long as the protein maintains a desired activity. For example, a serine residue may be substituted to eliminate a single reactive cysteine or to remove disulfide bonding or a conservative amino acid substitution may be made to eliminate a cleavage site. These modifications may be deliberate, as through site-directed mutagenesis, or may be accidental, such as through mutations of hosts, which produce the proteins or errors due to polymerase chain reaction (PCR) amplification.

As used herein, the term “wildtype” refers to a non-mutated version of a gene, allele, genotype, polypeptide, or phenotype, or a fragment of any of these. It may occur in nature or be produced recombinantly. As used herein, the term “variant” refers to a nucleic acid molecule or polypeptide that differs from a referent nucleic acid molecule or polypeptide by single or multiple amino acid substitutions, deletions, and/or additions and substantially retains at least one biological activity of the referent nucleic acid molecule or polypeptide.

The terms “peptide mimetic” or “peptidomimetic” refer to a peptide-like molecule that emulates a sequence derived from a protein or peptide. A peptide mimetic or peptidomimetic may contain amino acids and/or non-amino acid components. Examples of peptidomimetics include chemically modified peptides, peptoids (side chains are appended to the nitrogen atom of the peptide backbone, rather than to the α-carbons), P-peptides (amino group bonded to the β carbon rather than the α carbon), etc.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another: (1) Alanine (A) and Glycine (G); (2) Aspartic acid (D) and Glutamic acid (E); (3) Asparagine (N) and Glutamine (Q); (4) Arginine (R) and Lysine (K); (5) Isoleucine (I). Leucine (L), Methionine (M), and Valine (V); (6) Phenylalanine (F), Tyrosine (Y), and Tryptophan (W); (7) Serine(S) and Threonine (T); and (8) Cysteine (C) and Methionine (M).

Naturally occurring residues may be divided into classes based on common side chain properties, for example: polar positive (histidine (H), lysine (K), and arginine (R)); polar negative (aspartic acid (D), glutamic acid (E)); polar neutral (serine(S), threonine (T), asparagine (N). glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs. Non-conservative substitutions may involve the exchange of a member of one class for a member from another class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects.

“Treatment,” as used herein, covers any administration or application of a cosmetic or a therapeutic for disease, in a mammal, including a human, and includes inhibiting the disease, arresting its development, or relieving the disease, for example, by causing regression, or restoring or repairing a lost, missing, or defective function; or stimulating an inefficient process.

As used herein, “clinical improvement” may refer to a noticeable reduction in the symptoms of a disorder, or cessation thereof.

The terms “manage,” “managing,” and “management” refer to preventing or slowing the progression, spread or worsening of a disease or disorder, or of one or more symptoms thereof. In certain cases, the beneficial effects that a subject derives from a prophylactic or therapeutic agent do not result in a cure of the disease or disorder.

The terms “regress,” “regressing,” and “regression” may refer to a decrease in the size of a tumor or in the extent of cancer in the body. In some embodiments, “regression” may refer to a decrease in severity of the disease and/or decrease in the size of a tumor. In some embodiments, regression may generally refer to lighter symptoms without the disease completely disappearing. In certain cases, the beneficial effects that a subject derives from a prophylactic or therapeutic agent do not result in a cure of the disease or disorder. In some embodiments, symptoms of the disease may return.

The terms “administering” and “administration” refer to methods of providing a pharmaceutical preparation, composition, or formulation to a subject. The compositions described herein can be administered in a number of ways depending on whether local or systemic treatment is desired, and on the area to be treated. Such methods are well known to those skilled in the art and include, but are not limited to, administering the compositions orally, intranasally, parenterally (e.g., intravenously and subcutaneously), by intramuscular injection, by intraperitoneal injection, intrathecally, transdermally, extracorporeally, topically or the like.

As described above, the compositions can be administered to a subject in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

A “pharmaceutically acceptable carrier” refers to a non-toxic solid, semisolid, or liquid filler, diluent, encapsulating material, formulation auxiliary, or carrier conventional in the art for use with a therapeutic agent for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

The pharmaceutical formulation can be administered in a number of ways depending on whether local or systemic treatment is desired, and on the area to be treated. A preferred mode of administration of the composition is parenterally, for example by intramuscular injection. Other modes of administration may be used. The disclosed compounds can be locally administered by e.g., intramuscular routes of administration, such as by injection or use of an implant.

Preparations for parenteral administration include sterile aqueous or non-aqueous solutions, suspensions, and emulsions. Examples of non-aqueous solvents are propylene glycol, polyethylene glycol, vegetable oils such as olive oil, fish oils, and injectable organic-esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like. Another approach for parenteral administration involves use of a slow release or sustained release system such that a constant dosage is maintained

A “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days, weekly, twice weekly, etc. Guidance can be found in the literature for appropriate dosages for given classes of pharmaceutical products.

The exact amount of the compositions required will vary from subject to subject, depending on the species, age, weight, and general condition of the subject, the severity of the disorder being treated, the particular composition used, its mode of administration and the like. Thus, it is not possible to specify an exact amount for every composition. However, an appropriate amount can be determined by one of ordinary skill in the art using only routine experimentation given the teachings herein.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

The present invention addresses the comparatively lower toxicity of BoNT/E compared to BoNT/A (Botox), attributed in part to the reduced binding affinity of H_{C}E to SV2 relative to H_{C}A. By enhancing BoNT/E's capacity to recognize neuronal receptors SV2, the invention aims to reduce the required dosage for both cosmetic and therapeutic applications. The heightened affinity of the refined BoNT/E variant may help to expedite absorption into motor neurons at the injection site. This reduction in dosage not only minimizes undesirable toxin dispersion from the injection site but also mitigates the risk of immune responses that could compromise the efficacy of future treatments.

As described herein, H_{C}E exhibits recognition of SV2A through dual interfaces: the C-terminal sub-domain of H_{C}E establishes extensive hydrogen bonds and hydrophobic interactions with SV2A (termed the protein interface), while the previously undiscovered glycan-binding pocket within the N-terminal sub-domain of H_{C}E interacts with an N-linked glycan on SV2A (referred to as the glycan interface) (see Example 1). Notably, HE employs an analogous binding mode for SV2B. The present invention provides a thorough identification of the amino acids within H_{C}E pivotal for SV2A/2B binding, thereby presenting actionable targets for protein engineering pursuits. These endeavors are geared towards modulating the binding affinity to SV2A/2B as well as precisely refining binding specificity across SV2A, 2B, and 2C, offering promising avenues for enhancing therapeutic efficacy.

In some embodiments, the protein interface, e.g., the BoNT/E amino acids that interact with the protein-moiety of SV2A, comprises a combination of amino acids including R1100, K1102, A1154, T1157, H1158, L1159, F1160. In some embodiments, the glycan interface, e.g., BoNT/E amino acids that interact with the glycan-moiety of SV2A, comprises a combination of amino acids including: Y879, Y881, Y891, R922, N988, Y1041, H1247, G1248.

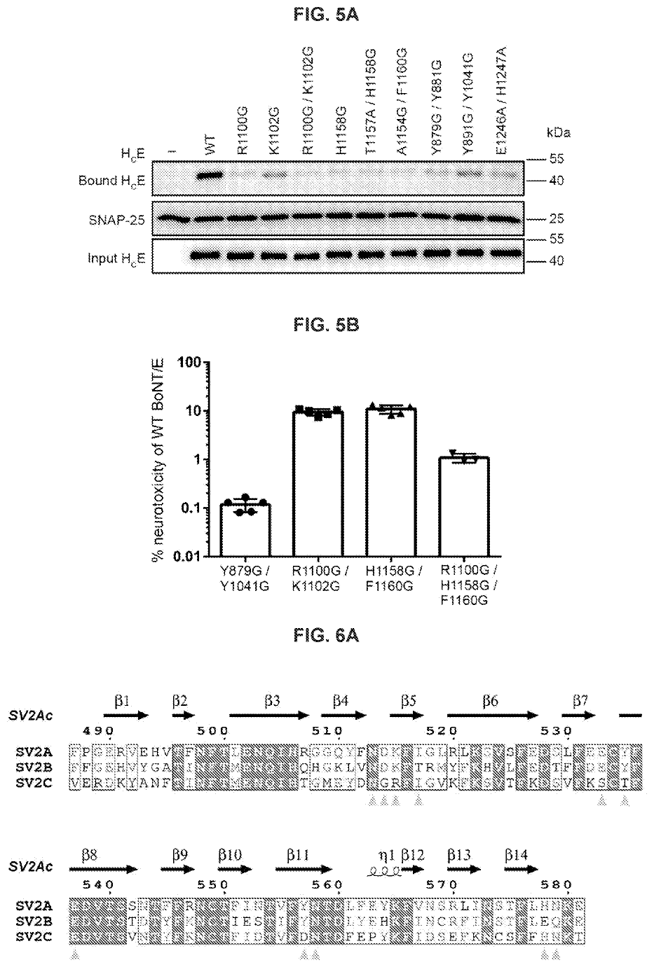

Referring to FIG. 5B, mutating a portion of these aforementioned SV2-binding residues in BoNT/E drastically decreased BoNT/E neurotoxicity. For example, BoNT/E (R1100G/K1102G) and BoNT/E (H1158G/F1160G) that carry mutations at the protein interface showed ~90% decreased toxicity, while BoNT/E (R1100G/H1158G/F1160G) showed a further decrease to ~99%. In some embodiments, endogenous BoNT/E-like SV2 binding is knocked out (e.g., using the mutations R1100G/H1158G/F1160G). Without wishing to limit the present invention to any theory or mechanism it is believed that this is necessary because the endogenous BoNT/E-like SV2 binding site, if not removed, may compete and interfere with the engineered BoNT/A-like SV2 binding site, and thus decrease binding efficiency. Furthermore, BoNT/E^{Y879G/Y1041G} that carries mutations at the glycan interface only retained ~0.1% neurotoxicity despite its intact binding site for the protein moiety of SV2A, strongly supporting the direct involvement of the N-glycan of SV2A and SV2B in BoNT/E binding and function.

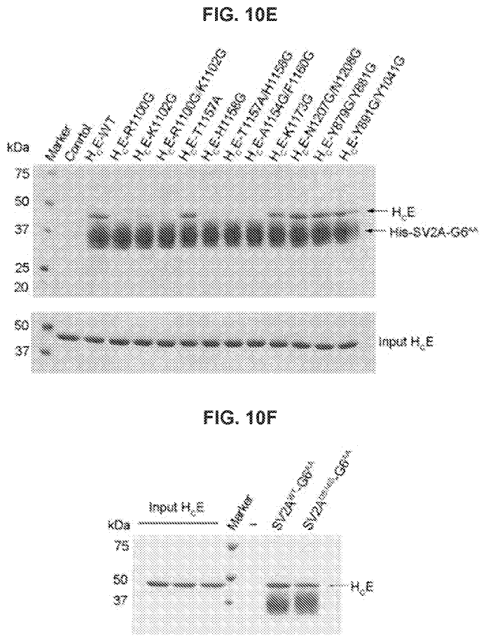

The present invention features a modified serotype E botulinum neurotoxin (BoNT/E) comprising a plurality of mutations. The plurality of mutations may decrease endogenous BoNT/E binding function. Additionally, the plurality of mutations may mimic endogenous botulinum neurotoxin A (BoNT/A) receptor binding. For example, in some embodiments, the modified BoNT/E may comprise one or more mutations (see FIG. 10E or FIG. 5A). In some embodiments, the modified BoNT/E may comprise two or more mutations. In some embodiments, the modified BoNT/E may comprise three or more mutations. In some embodiments, the modified BoNT/E may comprise four or more mutations. In some embodiments, the modified BoNT/E may comprise five or more mutations. In some embodiments, the modified BoNT/E may comprise ten or more mutations. In some embodiments, the modified BoNT/E may comprise twenty or more mutations. The present invention is not limited to the aforementioned mutations and may include any number of mutations that either i) decreases endogenous BoNT/E binding function or ii) mimics endogenous BoNT/A receptor binding.

In some embodiments, the present invention features a modified BoNT/E comprising one or more mutations. In some embodiments, the one or more mutations decrease endogenous BoNT/E binding function and/or mimic endogenous botulinum neurotoxin A (BoNT/A) receptor binding. In other embodiments, the present invention features a modified BoNT/E comprising two or more mutations. In some embodiments, the two or more mutations decrease endogenous BoNT/E binding function and/or mimic endogenous BoNT/A receptor binding. In further embodiments, the present invention features a modified BoNT/E comprising five or more mutations. In some embodiments, the five or more mutations decrease endogenous BoNT/E binding function and/or mimic endogenous BoNT/A receptor binding. In some embodiments, the present invention features a modified BoNT/E comprising ten or more mutations. In some embodiments, the ten or more mutations decrease endogenous BoNT/E binding function and/or mimic endogenous BoNT/A receptor binding.

The present invention may feature a modified serotype E botulinum neurotoxin (BoNT/E) comprising a sequence according to SEQ ID NO: 2, SEQ ID NO: 3, or SEQ ID NO: 4.

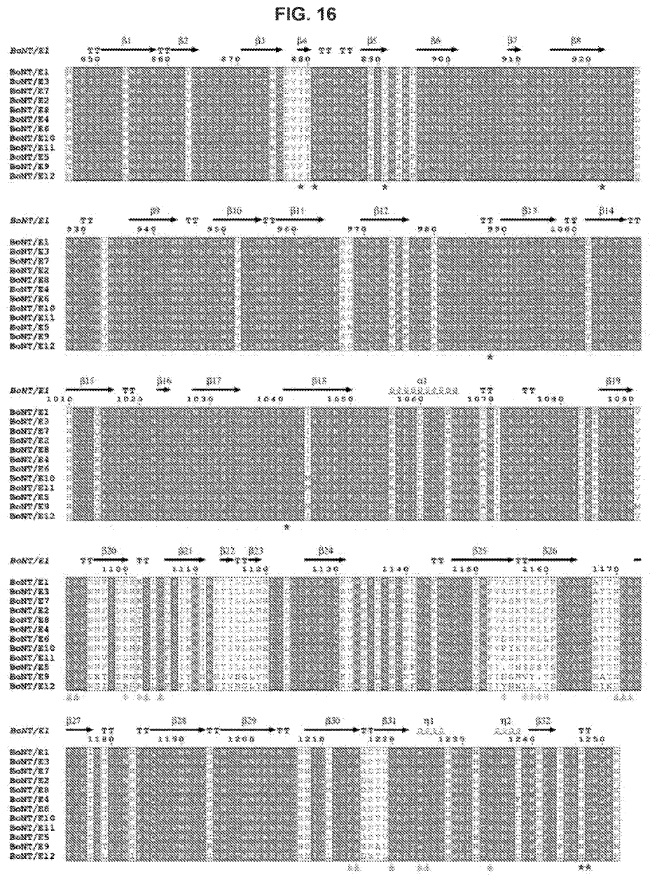

In some embodiments, the modified serotype E botulinum neurotoxin (BoNT/E) is a modified serotype E botulinum neurotoxin subtype 1 (BoNT/E1). In other embodiments, the modified BoNT/E is a modified BoNT/E2, a modified BoNT/E3, a modified BoNT/E4, a modified BoNT/E5, a modified BoNT/E6, a modified BoNT/E7, a modified BoNT/E8, a modified BoNT/E9, a modified BoNT/E10, a modified BoNT/E11, or a modified BoNT/E12. The present invention is not limited to use modifications to the BoNT/E subtype 1 (E1), and may include modifications of any of the aforementioned subtypes. For example, referring to FIG. 16, the modified BoNT/E of any of the aforementioned subtypes may comprise a plurality (e.g., one or more, or two or more, etc . . . ) of homologous mutations. One of ordinary skill in the art would understand which amino acids in E2-E12 are equivalent to the amino acids in E1 and thus be able to make appropriate non-conservative mutations as described herein.

In some embodiments, the one or more mutation that decreases endogenous BoNT/E binding function inhibits binding of BoNT/E to a receptor. The mutations that inhibit binding of BoNT/E to a receptor may comprise mutations at R1100, H1158, F1160, or a combination thereof. In some embodiments, the mutations that inhibit binding of BoNT/E to a receptor may comprise R1100G, H1158G, F1160G, or a combination thereof. However, the present invention is not limited to the mutations mentioned above (e.g., mutations exclusively to a glycine), but rather, it encompasses any non-conservative mutations.

In some embodiment, the one or more mutations that decrease endogenous BoNT/E receptor binding function comprise mutations at a protein interface, a glycan interface, or a combination thereof. In some embodiments, the mutation at the protein interface comprises mutations at R1100, K1102, A1154, T1157, H1158, L1159, F1160, or a combination thereof. In some embodiments, the mutation at the glycan interface comprises mutations at Y879, Y881, Y891, R922, N988, Y1041, H1247, G1248, or a combination thereof.

In some embodiment, the one or more mutations that mimic the endogenous BoNT/A receptor binding comprise mutations at the BoNT/A-like protein interface, BoNT/A-like glycan interface, or a combination thereof (see sequences in Table 1).

In some embodiments, the receptor comprises a synaptic vesicle glycoprotein 2 (SV2), and may additionally include the three isoforms of the receptor, e.g., SV2A, 2B, and 2C.

In some embodiments, the plurality of mutations increases binding affinity of the modified BoNT/E compared to a wild type BoNT/E. In some embodiments, the plurality of mutations modulates receptor binding specificity of the modified BoNT/E compared to a wild type BoNT/E.

TABLE 1

| | | SEQ ID |
|---|---|---|
| | Sequence | NO: |
| Wild type H_{C}E | RIKSSSVLNMRYKNDKYVDTSGYDSNININGDVYKYP | 1 |
| | TNKNQFGIYNDKLSEVNISQNDYIIYDNKYKNFSISFW | |
| | VRIPNYDNKIVNVNNEYTIINCMRDNNSGWKVSLNHN | |
| | EIIWTLQDNAGINQKLAFNYGNANGISDYINKWIFVTIT | |
| | NDRLGDSKLYINGNLIDQKSILNLGNIHVSDNILFKIVNC | |
| | SYTRYIGIRYFNIFDKELDETEIQTLYSNEPNTNILKDF | |
| | WGNYLLYDKEYYLLNVLKPNNFIDRRKDSTLSINNIRS | |
| | TILLANRLYSGIKVKIQRVNNSSTNDNLVRKNDQVYINF | |
| | VASKTHLFPLYADTATTNKEKTIKISSSGNRFNQVVVM | |
| | NSVGNNCTMNFKNNNGNNIGLLGFKADTVVASTWYY | |
| | THMRDHTNSNGCFWNFISEEHGWQEK | |
| H_{C}E-V1 | RIKSSSVLNMRYKNDKYVDTSGYDSNININGDVYKYP | 2 |
| (Bolded residues are the | TNKNQFGIYNDKLSEVNISQNDYIIYDNKYKNFSISFW | |
| ones that were changed | VRIPKYFNSISLNNEYTIINCMRDNNSGWKVSLNYGEII | |
| compared to the WT in | WTLQDNAGINQKLAFNYSQMINISDYINKWIFVTITND | |
| order to enhance | RLGDSKLYINGNLIDQKSILNLGNIHVSDNILFKIVNCRD | |
| protein-mediated receptor | THRYIGIRYFNIFDKELDETEIQTLYSNEPNTNILKDFW | |
| binding. Underlined | GNYLLYDKEYYLLNVLDPNKYIDRRKDSTLSIKGPRGS | |
| residues could affect both | VMTTNIYLNSSLYRGIKVKIQRVNNSSTNDNLVRKNDQ | |
| the protein- and | VYINFVASKTHLFPLYADTATTNKEKTIKISSSGNRFNQ | |
| glycan-mediated receptor | WVMNSVGNNCTMNFKNNNGNNIGLLGFKADTVVAS | |
| binding.) | TWYYTHMRDHTNSNGCFWNFISEEHGWGERPL | |
| H_{C}E-V3.5 | RIKSSSVLNMRYKNDKYVDTSGYDSNINIGSKVNKYP | 3 |
| (Bolded residues are the | TNKNQFQLFNLESSEVNISQNDYIIYDNKYKNFSISFW | |
| ones to improve | VRIPKYFNSISLNNEYTIINCMRDNNSGWKVSLNYGEII | |
| protein-mediated binding; | WTLQDNAGINQKLAFNYSQMINISDYINKWIFVTITND | |
| Italicized residues are the | RLGDSKLYINGNLIDQKSILNLGNIHVSDNILFKIVNCRD | |
| ones to improve | THRYIGIRYFNIFDKELDETEIQTLYSNEPNTNILKDFW | |
| glycan-mediated binding; | GNYLLYDKEYYLLNVLKPNNYIDGRKDSTLSIKGPRGS | |
| Underlined residues could | VMTTNIYLNSSLYRGIKVKIQRVNNSSTNDNLVRKNDQ | |
| affect both the protein- | VYINFVASKTGLGPLYADTATTNKEKTIKISSSGNRFNQ | |
| and glycan-mediated | WVMNSVGNNCTMNFKNNNGNNIGLLGFKADTVVAS | |
| receptor binding. The | TWYYTHMRDHTNSNGCFWNFISEDDGWGERPL | |
| three Gs Bolded and | | |
| underlined are the ones | | |
| to disrupt the endogenous | | |
| SV2-binding interface in | | |
| H_{C}E) | | |
| BoNT/E-V3.5 | MPKINSFNYNDPVNDRTILYIKPGGCQEFYKSFNIMKN | 4 |
| Engineered BoNT/E | IWIIPERNVIGTTPQDFHPPTSLKNGDSSYYDPNYLQS | |
| holotoxin. Underlined | DEEKDRFLKIVTKIFNRINNNLSGGILLEELSKANPYLG | |
| residues are the LC-H_{N} | NDNTPDNQFHIGDASAVEIKFSNGSQDILLPNVIIMGA | |
| domain of the wild-type | EPDLFETNSSNISLRNNYMPSNHGFGSIAIVTFSPEYS | |
| BoNT/E1 and the Bolded | FRFNDNSMNEFIQDPALTLMHELIHSLHGLYGAKGITT | |
| are the sequences of H_{C}E | KYTITQKQNPLITNIRGTNIEEFLTFGGTDLNIITSAQSN | |
| (V3.5; SEQ ID NO: 3). | DIYTNLLADYKKIASKLSKVQVSNPLLNPYKDVFEAKY | |
| | GLDKDASGIYSVNINKFNDIFKKLYSFTEFDLATKFQVK | |
| | CRQTYIGQYKYFKLSNLLNDSIYNISEGYNINNLKVNF | |
| | RGQNANLNPRIITPITGRGLVKKIIRFCKNIVSVKGIRKS | |
| | ICIEINNGELFFVASENSYNDDNINTPKEIDDTVTSNNN | |
| | YENDLDQVILNFNSESAPGLSDEKLNLTIQNDAYIPKY | |
| | DSNGTSDIEQHDVNELNVFFYLDAQKVPEGENNVNLT | |
| | SSIDTALLEQPKIYTFFSSEFINNVNKPVQAALFVSWIQ | |
| | QVLVDFTTEANQKSTVDKIADISIVVPYIGLALNIGNEA | |
| | QKGNFKDALELLGAGILLEFEPELLIPTILVFTIKSFLGS | |
| | SDNKNKVIKAINNALKERDEKWKEVYSFIVSNWMTKI | |
| | NTQFNKRKEQMYQALQNQVNAIKTIIESKYNSYTLEE | |
| | KNELTNKYDIKQIENELNQKVSIAMNNIDRFLTESSISY | |
| | LMKLINEVKINKLREYDENVKTYLLNYIIQHGSILGESQ | |
| | QELNSMVTDTLNNSIPFKLSSYTDDKILISYFNKFFKRI | |
| | KSSSVLNMRYKNDKYVDTSGYDSNINIGSKVNKYPT | |
| | NKNQFQLFNLESSEVNISQNDYIIYDNKYKNFSISFWV | |
| | RIPKYFNSISLNNEYTIINCMRDNNSGWKVSLNYGEII | |
| | WTLQDNAGINQKLAFNYSQMINISDYINKWIFVTITND | |
| | RLGDSKLYINGNLIDQKSILNLGNIHVSDNILFKIVNCR | |
| | DTHRYIGIRYFNIFDKELDETEIQTLYSNEPNTNILKDF | |
| | WGNYLLYDKEYYLLNVLKPNNYIDGRKDSTLSIKGPR | |
| | GSVMTTNIYLNSSLYRGIKVKIQRVNNSSTNDNLVRK | |
| | NDQVYINFVASKTGLGPLYADTATTNKEKTIKISSSGN | |
| | RFNQVVVMNSVGNNCTMNFKNNNGNNIGLLGFKAD | |
| | TVVASTWYYTHMRDHTNSNGCFWNFISEDDGWGER | |
| | PL | |

In some embodiments, SEQ ID NO: 3 (e.g., H_{C}E-V3.5) as described herein may be used to replace the H_{C} of any of the BoNT/E subtypes described herein (e.g., BoNT/E2 (SEQ ID NO: 5). BoNT/E3 (SEQ ID NO: 6), BoNT/E4 (SEQ ID NO: 7), BoNT/E5 (SEQ ID NO: 8). BoNT/E6 (SEQ ID NO: 9), BoNT/E7 (SEQ ID NO: 10), BoNT/E8 (SEQ ID NO: 11), BoNT/E9 (SEQ ID NO: 12), BoNT/E10 (SEQ ID NO: 13), BoNT/E11 (SEQ ID NO: 14), BoNT/E12 (SEQ ID NO: 15)). Without wishing to limit the present invention to any particular theory or mechanism, it is believed that replacing the H_{C} domain of any of the aforementioned subtypes may give these toxins the unique receptor-binding features of H_{C}E-V3.5.

Table 2 shows representative sequences for chimeric designs of BoNT/E2-E12 with their receptor-binding domain replaced with H_{C}E-V3.5. The underlined sequences are that of the wild-type LC-H_{N} fragment of the indicated BoNT/E subtype, and the bolded sequence is H_{C}E-V3.5. (BoNT/E2 (SEQ ID NO: 5) BoNT/E3, BoNT/E4

TABLE 2

| | | SEQ |
|---|---|---|
| | Sequence | ID NO: |
| BoNT/E2 | MPKINSFNYNDPVNDRTILYIKPGGCQEFYKSENIMKNIWIIPE | 5 |
| | RNVIGTTPQDFHPPTSLKNGDSSYYDPNYLQSDEEKDRFLKI | |
| | VTKIFNRINNNLSGGILLEELSKANPYLGNDNTPDNQFHIGDA | |
| | SAVEIKFSNGIQDILLPNVIIMGAEPDLFETNSSNISLRNNYMP | |
| | SNHGFGSIAIVTFSPEYSFRFNDNSMNEFIQDPALTLMHELIH | |
| | SLHGLYGAKGITTKYTITQKQNPLITNIRGTNIEEFLTFGGTDL | |
| | NIITSAQSNDIYTNLLADYKKIASKLSKVQVSNPLLNPYKDVFE | |
| | AKYGLDKDASGIYSVNINKFNDIFKKLYSFTEFDLATKFQVKC | |
| | RQTYIGQYKYFKLSNLLNDSIYNISEGYNINNLKVNFRGQNAN | |
| | LNPRIITPITGRGLVKKIIRFCKNIVSVKGIRKSICIEINNGELFFV | |
| | ASENSYNDDNINTPKEIDDTVTSNNNYENDLDQVILNFNSES | |
| | APGLSDEKLNLTIQNDAYIPKYDSNGTSDIEQHDVNELNVFFY | |
| | LDAQKVPEGENNVNLTSSIDTALLEQPKIYTFFSSEFINNVNK | |
| | PVQAALFVSWIQQVLVDFTTEANQKSTVDKIADISIVVPYIGLA | |
| | LNIGNEAQKGNEKDALELLGAGILLEFEPELLIPTILVFTIKSFL | |
| | GSSDNKNKVIKAINNALKERDEKWKEVYSFIVSNWMTKINTQ | |
| | FNKRKEQMYQALQNQVNAIKTIIESKYNSYTLEEKNELTNKYD | |
| | IKQIENELNQKVSIAMNNIDRFLTESSISYLMKLINEVKINKLRE | |
| | YDENVKTYLLNYIIQHGSILGESQQELNSMVTDTLNNSIPFKL | |
| | SSYTDDKILISYFNKFFKRIKSSSVLNMRYKNDKYVDTSGYD | |
| | SNINIGSKVNKYPTNKNQFQLFNLESSEVNISQNDYIIYDNKY | |
| | KNFSISFWVRIPKYFNSISLNNEYTIINCMRDNNSGWKVSLN | |
| | YGEIIWTLQDNAGINQKLAFNYSQMINISDYINKWIFVTITNDR | |
| | LGDSKLYINGNLIDQKSILNLGNIHVSDNILFKIVNCRDTHRYI | |
| | GIRYFNIFDKELDETEIQTLYSNEPNTNILKDFWGNYLLYDKE | |
| | YYLLNVLKPNNYIDGRKDSTLSIKGPRGSVMTTNIYLNSSLY | |
| | RGIKVKIQRVNNSSTNDNLVRKNDQVYINFVASKTGLGPLYA | |
| | DTATTNKEKTIKISSSGNRFNQVVVMNSVGNNCTMNFKNNN | |
| | GNNIGLLGFKADTVVASTWYYTHMRDHTNSNGCFWNFISE | |
| | DDGWGERPL | |
| BoNT/E3 | MPKINSFNYNDPVNDRTILYIKPGGCQEFYKSFNIMKNIWIIPE | 6 |
| | RNVIGTTPQDFHPPTSLKNGDSSYYDPNYLQSDEEKDRFLKI | |
| | VTKIFNRINNNLSGGILLEELSKANPYLGNDNTPDNQFHIGDA | |
| | SAVEIKFSNGSQHILLPNVIIMGAEPDLFETNSSNISLRNNYMP | |
| | SNHGFGSIAIVTFSPEYSFRFNDNSINEFIQDPALTLMHELIHS | |
| | LHGLYGAKGITTTCIITQQQNPLITNRKGINIEEFLTFGGNDLNII | |
| | TVAQYNDIYTNLLNDYRKIASKLSKVQVSNPQLNPYKDIFQEK | |
| | YGLDKDASGIYSVNINKFDDILKKLYSFTEFDLATKFQVKCRE | |
| | TYIGQYKYFKLSNLLNDSIYNISEGYNINNLKVNFRGQNANLN | |
| | PRIIKPITGRGLVKKIIRFCKNIVSVKGIRKSICIEINNGELFFVAS | |
| | ENSYNDDNINTPKEIDDTVTSNNNYENDLDQVILNFNSESAP | |
| | GLSDEKLNLTIQNDAYIPKYDSNGTSDIEQHDVNELNVEFYLD | |
| | AQKVPEGENNVNLTSSIDTALLEQPKIYTFFSSEFINNVNKPV | |
| | QAALFVSWIQQVLVDFTTEANQKSTVDKIADISIVVPYIGLALN | |
| | IGNEAQKGNEKDALELLGAGILLEFEPELLIPTILVFTIKSFLGS | |
| | SDNKNKVIKAINNALKERDEKWKEVYSFIVSNWMTKINTQFN | |
| | KRKEQMYQALQNQVNAIKTIIESKYNSYTLEEKNELTNKYDIK | |
| | QIENELNQKVSIAMNNIDRFLTESSISYLMKLINEVKINKLREY | |
| | DENVKTYLLNYIIQHGSILGESQQELNSMVTDTLNNSIPFKLS | |
| | SYTDDKILISYFNKFFKRIKSSSVLNMRYKNDKYVDTSGYDS | |
| | NINIGSKVNKYPTNKNQFQLFNLESSEVNISQNDYIIYDNKYK | |
| | NFSISFWVRIPKYFNSISLNNEYTIINCMRDNNSGWKVSLNY | |
| | GEIIWTLQDNAGINQKLAFNYSQMINISDYINKWIFVTITNDRL | |
| | GDSKLYINGNLIDQKSILNLGNIHVSDNILFKIVNCRDTHRYIGI | |
| | RYFNIFDKELDETEIQTLYSNEPNTNILKDFWGNYLLYDKEYY | |
| | LLNVLKPNNYIDGRKDSTLSIKGPRGSVMTTNIYLNSSLYRGI | |
| | KVKIQRVNNSSTNDNLVRKNDQVYINFVASKTGLGPLYADTA | |
| | TTNKEKTIKISSSGNRFNQVVVMNSVGNNCTMNFKNNNGN | |
| | NIGLLGFKADTVVASTWYYTHMRDHTNSNGCFWNFISEDD | |
| | GWGERPL | |
| BoNT/E4 | MPTINSFNYNDPVNNRTILYIKPGGCQQFYKSENIMKNIWIIPE | 7 |
| | RNVIGTIPQDFLPPTSLKNGDSSYYDPNYLQSDQEKDKFLKIV | |
| | TKIFNRINDNLSGRILLEELSKANPYLGNDNTPDGDFIINDASA | |
| | VPIQFSNGSQSILLPNVIIMGAEPDLFETNSSNISLRNNYMPS | |
| | NHGFGSIAIVTFSPEYSFRFKDNSMNEFIQDPALTLMHELIHSL | |
| | HGLYGAKGITTKYTITQKQNPLITNIRGTNIEEFLTFGGTDLNII | |
| | TSAQSNDIYTNLLADYKKIASKLSKVQVSNPLLNPYKDVFEAK | |
| | YGLDKDASGIYSVNINKFNDIFKKLYSFTEFDLATKFQVKCRQ | |
| | TYIGQYKYFKLSNLLNDSIYNISEGYNINNLKVNFRGQNANLN | |
| | PRIITPITGRGLVKKIIRFCKNIVSVKGIRKSICIEINNGELFFVAS | |
| | ENSYNDDNINTPKEIDDTVTSNNNYENDLDQVILNFNSESAP | |
| | GLSDEKLNLTIQNDAYIPKYDSNGTSDIEQHDVNELNVEFYLD | |
| | AQKVPEGENNVNLTSSIDTALLEQPKIYTFFSSEFINNVNKPV | |
| | QAALFVGWIQQVLVDETTEANQKSTVDKIADISIVVPYIGLALN | |
| | IGNEAQKGNEKDALELLGAGILLEFEPELLIPTILVFTIKSFLGS | |
| | SDNKNKVIKAINNALKERDEKWKEVYSFIVSNWMTKINTQFN | |
| | KRKEQMYQALQNQVNALKAIIESKYNSYTLEEKNELTNKYDIE | |
| | QIENELNQKVSIAMNNIDRFLTESSISYLMKLINEVKINKLREY | |
| | DENVKTYLLDYIIKHGSILGESQQELNSMVIDTLNNSIPFKLSS | |
| | YTDDKILISYENKFFKRIKSSSVLNMRYKNDKYVDTSGYDSNI | |
| | NIGSKVNKYPTNKNQFQLFNLESSEVNISQNDYIIYDNKYKN | |
| | FSISFWVRIPKYFNSISLNNEYTIINCMRDNNSGWKVSLNYG | |
| | EIIWTLQDNAGINQKLAFNYSQMINISDYINKWIFVTITNDRLG | |
| | DSKLYINGNLIDQKSILNLGNIHVSDNILFKIVNCRDTHRYIGIR | |
| | YFNIFDKELDETEIQTLYSNEPNTNILKDFWGNYLLYDKEYYL | |
| | LNVLKPNNYIDGRKDSTLSIKGPRGSVMTTNIYLNSSLYRGIK | |
| | VKIQRVNNSSTNDNLVRKNDQVYINFVASKTGLGPLYADTAT | |
| | TNKEKTIKISSSGNRFNQVVVMNSVGNNCTMNFKNNNGNNI | |
| | GLLGFKADTVVASTWYYTHMRDHTNSNGCFWNFISEDDGW | |
| | GERPL | |
| BoNT/E5 | MPKINSFNYNDPVNDRTILYIKPGGCQEFYKSFNIMKNIWIIPE | 8 |
| | RNVIGTTPQDFHPPTSLKNGDSSYYDPNYLQSDEEKDRFLKI | |
| | VTKIFNRINNNLSGGILLEELSKANPYLGNDNTPDNQFHIGDA | |
| | SAVEIKFSNGSQDILLPNVIIMGAEPDLFETNSSNISLRNNYMP | |
| | SNHGFGSIAIVTFSPEYSFRFNDNSMNEFIQDPALTLMHELIH | |
| | SLHGLYGAKGITTKYTITQKQNPLITNIRGTNIEEFLTFGGTDL | |
| | NIITSAQSNDIYTNLLADYKKIASKLSKVQVSNPLLNPYKDVFE | |
| | AKYGLDKDASGIYSVNINKFNDIFKKLYSFTEFDLATKFQVKC | |
| | RQTYIGQYKYFKLSNLLNDSIYNISEGYNINNLKVNFRGQNAN | |
| | LNPRIITPITGRGLVKKIIRFCKNIVSVKGIRKSICIEINNGELFFV | |
| | ASENSYNDDNINTPKEIDDTVTSNNNYENDLDQVILNFNSES | |
| | APGLSDEKLNLTIQNDAYIPKYDSNGTSDIEQHDVNELNVFFY | |
| | LDAQKVPEGENNVNLTSSIDTALLEQPKIYTFFSSEFINNVNK | |
| | PVQAALFVSWIQQVLVDFTTEANQKSTVDKIADISIVVPYIGLA | |
| | LNIGNEAQKGNFKDALELLGAGILLEFVPELLIPTILVFTIKSFL | |
| | GSSDNKNKVIKAINNALKERDEKWKEVYSFIVSNWMTKINTQ | |
| | FNKRKEQMYQALQNQVNALKTIIEFKYNSYTLEEKKELKNNY | |
| | DIEQIENELNQKVSIAMNNIDRFLTESSISYLMKLINEVKINKLR | |
| | EYDENVKTYLLDYIIQHGSILGESQQELNSMVIDTLNNSIPFKL | |
| | SSYTDDKILISYFNKFFKRIKSSSVLNMRYKNDKYVDTSGYD | |
| | SNINIGSKVNKYPTNKNQFQLFNLESSEVNISQNDYIIYDNKY | |
| | KNFSISFWVRIPKYFNSISLNNEYTIINCMRDNNSGWKVSLN | |
| | YGEIIWTLQDNAGINQKLAFNYSQMINISDYINKWIFVTITNDR | |
| | LGDSKLYINGNLIDQKSILNLGNIHVSDNILFKIVNCRDTHRYI | |
| | GIRYFNIFDKELDETEIQTLYSNEPNTNILKDFWGNYLLYDKE | |
| | YYLLNVLKPNNYIDGRKDSTLSIKGPRGSVMTTNIYLNSSLY | |
| | RGIKVKIQRVNNSSTNDNLVRKNDQVYINFVASKTGLGPLYA | |
| | DTATTNKEKTIKISSSGNRFNQVVVMNSVGNNCTMNFKNNN | |
| | GNNIGLLGFKADTVVASTWYYTHMRDHTNSNGCFWNFISE | |
| | DDGWGERPL | |
| BoNT/E6 | SYEEKDRELKIVTKIENRINNNLSGGILLEELSKANPYLGNDNT | 9 |
| | PDNQFHIGDASAVEIKFSNGSQDILLPNVIIMGAEPDLFETNS | |
| | SNISLRNNYMPSNHGFGSIAIVTFSPEYSFRFKDNSMNEFIQD | |
| | PALTLMHELIHSLHGLYGAKGITTQYTITQQQNPLITNIKGTNIE | |
| | EFLTFGGTDLNIITSAQYNDIYTNLLADYKKIASKLSKVQVSNP | |
| | LLNPYKDVFEKKYGLDKDASGIYSVNINKENDIFKKLYSFTEE | |
| | DLATKFQVKCRQTYIGQYKYFKLSNLLNNSIYNISEGYNINTLK | |
| | VNFRGQNTNLNPRIITPLTGRGLVKKIIRFCKNIVESKGIRKSICI | |
| | EINNGELFFVASDNSYNDDNINTPKEIDDTVTSNNNYENDLD | |
| | QVILNENSESAPGLSDEKLNLTIQNDAYIPKYDSNGTSDIEQH | |
| | DVNELNVFFYLDAQKVPEGENNIDFTSSIDTALLEQPKIYTFF | |
| | SSEFINNVNKPVQAALFVSWIQQVLVDETTEANQKSTVDKIA | |
| | DISIVVPYIGLALNIGNEAQKGNEKDALELLGAGILLEFEPELLI | |
| | PTILVFTIKSFLGSSDNKNKVIKAINNALKERDEKWKEVYSFIV | |
| | SNWMTKINTQENKRKEQMYQALQNQVNALKTIIESKYNSYTL | |
| | EEKNELTNKYNIEQIENELNQKVSIAMNNIEIFLTESSISYLMKL | |
| | INEVKINKLREYDENVKTYLLDYIIKHGSILGESQQELNSMVID | |
| | TLNNSIPFKLSSYTDDKILISYENKFFKRIKSSSVLNMRYKNDK | |
| | YVDTSGYDSNINIGSKVNKYPTNKNQFQLFNLESSEVNISQN | |
| | DYIIYDNKYKNFSISFWVRIPKYFNSISLNNEYTIINCMRDNNS | |
| | GWKVSLNYGEIIWTLQDNAGINQKLAFNYSQMINISDYINKW | |
| | IFVTITNDRLGDSKLYINGNLIDQKSILNLGNIHVSDNILFKIVN | |
| | CRDTHRYIGIRYFNIFDKELDETEIQTLYSNEPNTNILKDFWG | |
| | NYLLYDKEYYLLNVLKPNNYIDGRKDSTLSIKGPRGSVMTTN | |
| | IYLNSSLYRGIKVKIQRVNNSSTNDNLVRKNDQVYINFVASKT | |
| | GLGPLYADTATTNKEKTIKISSSGNRFNQVVVMNSVGNNCT | |
| | MNFKNNNGNNIGLLGFKADTVVASTWYYTHMRDHTNSNGC | |
| | FWNFISEDDGWGERPL | |
| BoNT/E7 | MPKINSFNYNDPVNDKTILYIKPGGCQQFYKSFNIMKNIWIIPE | 10 |
| | RNVIGTIPQDFLPPTSLKNGDSSYYDPNYLQSNEEKDRFLKIV | |
| | TKIFNRINDNLSGRILLEELSKANPYLGNDNTPDNQFHIGDAS | |
| | AVEIKFSNGNQSILLPNVIIMGAEPDLFETNSSNISLRNNYMPS | |
| | NHGFGSIAIVTFSPEYSFRFNDNSMNEFIQDPALTLMHELIHS | |
| | LHGLYGAKRITTKYTITQQQNPLITNIRGTNIEEFLTFGGTDLNI | |
| | ITSAQYNDIYTNLLADYKKIASKLSKVQVSNPQLNPYKDIFQE | |
| | KYGLDKNASGIYSVNINKFDDIFKKLYSFTEFDLATKFQVKCR | |
| | QTYIGQYKYFKLSNLLNNSIYNISEGYNINTLKVNFRGQNTNL | |
| | NPRIITQLTGRGLVKKIIRFCKNIVFSKGITKSICIEINNGELFFVA | |
| | SENSYNDDNINTPKEIDDTVTSNNNYENDLDQVILNFNSESA | |
| | PGLSDEKLNLTIQNDAYIPKYDSNGTSDIEQHDVNELNVFFYL | |
| | DAQKVPEGENNVNLTSSIDTALLEQPKIYTFFSSEFINNVNKP | |
| | VQAALFVSWIQQVLVDFTTEANQKSTVDKIADISIVVPYIGLAL | |
| | NIGNEAQKGNFKDALELLGAGILLEFEPELLIPTILVFTIKSFLG | |
| | SSDNKNKVIKAINNALKERDEKWKEVYSFIVSNWMTKINTQF | |
| | NKRKEQMYQALQNQVNAIKTIIESKYNSYTLEEKNELTNKYDI | |
| | KQIENELNQKVSIAMNNIDRFLTESSISYLMKLINEVKINKLRE | |
| | YDENVKTYLLNYIIQHGSILGESQQELNSMVTDTLNNSIPEKL | |
| | SSYTDDKILISYENKFFKRIKSSSVLNMRYKNDKYVDTSGYD | |
| | SNINIGSKVNKYPTNKNQFQLFNLESSEVNISQNDYIIYDNKY | |
| | KNFSISFWVRIPKYFNSISLNNEYTIINCMRDNNSGWKVSLN | |
| | YGEIIWTLQDNAGINQKLAFNYSQMINISDYINKWIFVTITNDR | |
| | LGDSKLYINGNLIDQKSILNLGNIHVSDNILFKIVNCRDTHRYI | |
| | GIRYFNIFDKELDETEIQTLYSNEPNTNILKDFWGNYLLYDKE | |
| | YYLLNVLKPNNYIDGRKDSTLSIKGPRGSVMTTNIYLNSSLY | |
| | RGIKVKIQRVNNSSTNDNLVRKNDQVYINFVASKTGLGPLYA | |
| | DTATTNKEKTIKISSSGNRFNQVVVMNSVGNNCTMNFKNNN | |
| | GNNIGLLGFKADTVVASTWYYTHMRDHTNSNGCFWNFISE | |
| | DDGWGERPL | |
| BoNT/E8 | ACEFMPKINSFNYNDPVNDKTILYIKPGGCQQFYKSFNIMKNI | 11 |
| | WIIPERNVIGTIPQDELPPTSLKNGDSSYYDPNYLQSNEEKDR | |
| | FLKIVTKIFNRINDNLSGRILLEELSKANPYLGNDNTPDNQFHI | |
| | GDASAVEIKFSNGNQSILLPNVIIMGAEPDLFETNSSNISLRNN | |
| | YMPSNHGFGSIAIVTFSPEYSFRFNDNSMNEFIQDPALTLMH | |
| | ELIHSLHGLYGAKRITTKYTITQQQNPLITNIRGTNIEEFLTFGG | |
| | TDLNIITSAQYNDIYTNLLADYKKIASKLSKVQVSNPQLNPYKD | |
| | IFQEKYGLDKNASGIYSVNINKFDDIFKKLYSFTEFDLATKFQV | |
| | KCRQTYIGQYKYFKLSNLLNNSIYNISEGYNINTLKVNFRGQN | |
| | TNLNPRIITQLTGRGLVKKIIRFCKNIVESKGITKSICIEINNGELF | |
| | FVASENSYNDDNINTPKEIDDTVTSNNNYENDLDQVILNENSE | |
| | SAPGLSDEKLNLTIQNDAYIPKYDSNGTSYIEQHDVNELNVFF | |
| | YLDAQKVPEGENNVNLTSSIDTALLEQPKIYTFFSSEFINNVN | |
| | KTVQAALFVSWIQQVLVDFTTEANQKSTVDKIADISIVVPYIGL | |
| | ALNIGNEAQKGNFKDALELLGAGILLEFEPELLIPTILVFTIKSF | |
| | LGSSDNKNKVIKAINNALKERDEKWKEVYSEIVSNWMTKINT | |
| | QFNKRKEQMYQALQNQVNALKTIIESKYNSYTLEEKNELTNK | |
| | YNIEQIENELNQKVSIAMNNIEIFLTESSISYLMKLINEVKINKLR | |
| | EYDENVKTYLLDYIIKHGSILGESQQELNSMVIDTLNNSIPFKL | |
| | SSYTDDKILISYENKFFKRIKSSSVLNMRYKNDKYVDTSGYD | |
| | SNINIGSKVNKYPTNKNQFQLFNLESSEVNISQNDYIIYDNKY | |
| | KNFSISFWVRIPKYFNSISLNNEYTIINCMRDNNSGWKVSLN | |
| | YGEIIWTLQDNAGINQKLAFNYSQMINISDYINKWIFVTITNDR | |
| | LGDSKLYINGNLIDQKSILNLGNIHVSDNILFKIVNCRDTHRYI | |
| | GIRYFNIFDKELDETEIQTLYSNEPNTNILKDFWGNYLLYDKE | |
| | YYLLNVLKPNNYIDGRKDSTLSIKGPRGSVMTTNIYLNSSLY | |
| | RGIKVKIQRVNNSSTNDNLVRKNDQVYINFVASKTGLGPLYA | |
| | DTATTNKEKTIKISSSGNRFNQVVVMNSVGNNCTMNFKNNN | |
| | GNNIGLLGFKADTVVASTWYYTHMRDHTNSNGCFWNFISE | |
| | DDGWGERPL | |
| BoNT/E9 | MPKINSFNYNDPVNDNTILYIKPGGCQQFYKSFNIMKNIWIIPE | 12 |
| | RNVIGTIPQNFLPPTSLKNGDSSYYDPNYLQNDQEKDRFLKI | |
| | VTKVFNRINDNLSGRILLEELSKANPYLGNDNTRDDDFIINDG | |
| | SAVPIQFSNGSQSILLPTVIIMGAEPDLFETNSSNVSLINNYSP | |
| | SNHGFGSIAIVTFSPEYSFRFNDNSMNEFIQDPALTLIHELIHS | |
| | LHGLYGAKGITTKYTITQQQNPLITNIRGINIEEFLTFGGNNLNII | |
| | TSSQLNDIYTNLLDDYKKIASKLSKVQVSNPQLNPYKDVFQE | |
| | KYGLDKNASGIYSVNINKFNDIFKKLYSFTEFDLATKFQVKCR | |
| | ETYIGQYKYFKLSNLLNDSIYNISEGYNINTLNVNFRGQNPNL | |
| | NPRIITPITDRGLVKKIIRFCKNIVSVKGIRKSICIEVNNGDLFFV | |
| | ASEKSYNNDSINIPKEIDDTVTLNNNYENDLDQVILNENSESA | |
| | PGLSDKKLNISIQDDVYIPKYDSNGTSDIEQYDVSELNVFFYL | |
| | DAQKVPEGENNVNLTSSIDTALLEQSKIYTFFSSEFINNVNKP | |
| | VQAALFVGWIQQVLVDETTEATQKSTVDKIADISIVVPYIGLAL | |
| | NIGNESQKGNFKDALELLGAGILLEFVPELLIPTILVFTIKSFLG | |
| | SSDNKNKVIKAINNALKERDEKWKEVYSFIVSNWITKINTQEN | |
| | KRKEQMYQALQNQVNALKTIESKYNSYTLEEKNELTNKYDIE | |
| | QIENELNQKVSIAMNNIDRFLTESSISYLMKLINEVKINKLREY | |
| | DENVKTYLLDYITKHGSILGESQQELNSMIIDTLNNSIPFKLSS | |
| | YTDDKILISYFNKFFKRIKSSSVLNMRYKNDKYVDTSGYDSNI | |
| | NIGSKVNKYPTNKNQFQLFNLESSEVNISQNDYIIYDNKYKN | |
| | FSISFWVRIPKYFNSISLNNEYTIINCMRDNNSGWKVSLNYG | |
| | EIIWTLQDNAGINQKLAFNYSQMINISDYINKWIFVTITNDRLG | |
| | DSKLYINGNLIDQKSILNLGNIHVSDNILFKIVNCRDTHRYIGIR | |
| | YFNIFDKELDETEIQTLYSNEPNTNILKDFWGNYLLYDKEYYL | |
| | LNVLKPNNYIDGRKDSTLSIKGPRGSVMTTNIYLNSSLYRGIK | |
| | VKIQRVNNSSTNDNLVRKNDQVYINFVASKTGLGPLYADTAT | |
| | TNKEKTIKISSSGNRFNQVVVMNSVGNNCTMNFKNNNGNNI | |
| | GLLGFKADTVVASTWYYTHMRDHTNSNGCFWNFISEDDGW | |
| | GERPL | |
| BoNT/E10 | MPKINSFNYNDPVNDKTILYIKPGGCQQFYKSFNIMKNIWIIPE | 13 |
| | RNVIGTIPQDFLPPTSLKNGDSSYYDPNYLQSNEEKDRELKIV | |
| | TKIFNRINDNLSGGILLEELSKANPYLGNDNTPNNQFHIGDAS | |
| | AVEIKFSNGSQSILLPTVIIMGAEPDLFETNSSNISLKNNYMPS | |
| | NHGFGSIAIVTFSPEYSFRFNDNSMNEFIQDPALTLMHELIHS | |
| | LHGLYGAKGITTKYTITQQQNPLITNIRGTNIEEFLTFGGTDLNI | |
| | ITNAQSNDIYTNLLADYKKIASKLSQVQVSNPQLNPYKDIFQE | |
| | KYGLDKNASGIYSVNINKFDDIFKKLYSFTEFDLATKFQVKCR | |
| | QTYIGQYKYFKLSNLLNNSIYNISEGYNINTLKVNFRGQNTNL | |
| | NPRIITQLTGRGLVKKIIRFCKNIVFSKGITKSICIEINNGELFFVA | |
| | SENSYNDDNINTPKEIDDTVTSNNNYENDLDQVILNENSESA | |
| | PGLSDEKLNLTIQNDAYIPKYDSNGTSDIEQHDVNELNVFFYL | |
| | DAQKVPEGENNVNLTSSIDTALLEQPKIYTFFSSEFINNVNKP | |
| | VQAALFVSWIQQVLVDFTTEANQKSTVDKIADISIVVPYIGLAL | |
| | NIGNEAQKGNFKDALELLGAGILLEFEPELLIPTILVFTIKSFLG | |
| | SSDNKNKVIKAINNALKERDEKWKEVYSFIVSNWMTKINTQF | |
| | NKRKEQMYQALQNQVNALKTIIESKYNSYTLEEKNELTNKYNI | |
| | EQIENELNQKVSIAMNNIEIFLTESSISYLMKLINEVKINKLREY | |
| | DENVKTYLLDYIIKHGSILGESQQELNSMVIDTLNNSIPFKLSS | |
| | YTDDKILISYFNKFFKRIKSSSVLNMRYKNDKYVDTSGYDSNI | |
| | NIGSKVNKYPTNKNQFQLFNLESSEVNISQNDYIIYDNKYKN | |
| | FSISFWVRIPKYFNSISLNNEYTIINCMRDNNSGWKVSLNYG | |
| | EIIWTLQDNAGINQKLAFNYSQMINISDYINKWIFVTITNDRLG | |
| | DSKLYINGNLIDQKSILNLGNIHVSDNILFKIVNCRDTHRYIGIR | |
| | YFNIFDKELDETEIQTLYSNEPNTNILKDFWGNYLLYDKEYYL | |
| | LNVLKPNNYIDGRKDSTLSIKGPRGSVMTTNIYLNSSLYRGIK | |
| | VKIQRVNNSSTNDNLVRKNDQVYINFVASKTGLGPLYADTAT | |
| | TNKEKTIKISSSGNRFNQVVVMNSVGNNCTMNFKNNNGNNI | |
| | GLLGFKADTVVASTWYYTHMRDHTNSNGCFWNFISEDDGW | |
| | GERPL | |
| BoNT/E11 | MPKINSFNYNDPVNDKTILYIKPGGCQQFYKSENIMKNIWIIPE | 14 |
| | RNVIGTIPQDFLPPTSLKNGDSSYYDPNYLQSNEEKDRELKIV | |
| | TKIFNRINDNLSGGILLEELSKANPYLGNDNTPNNQFHIGDAS | |
| | AVEIKFSNGSQSILLPTVIIMGAEPDLFETNSSNISLKNNYMPS | |
| | NHGFGSIAIVTFSPEYSFRFNDNSMNEFIQDPALTLMHELIHS | |
| | LHGLYGAKGITTKYTITQQQNPLITNIRGTNIEEFLTFGGTDLNI | |
| | ITNAQSNDIYTNLLDDYKKIASKLSQVQVSNPQLNPYKDVFQE | |
| | KYGLDKDANGIYSVNINKFNDIFKKLYSFTEFDLATKFQVKCR | |
| | KTYIGHHKYFRLSDLLNDSIYNISDGYNINTLKVNFRGQNTNL | |
| | NTRIITPITGRGVRKIIRFCTNIFSPKGIRKSICIEVNNGELFFV | |
| | ASENSYNDDNINTSKEIDDTVTSNNNYENDLDQVILNFNSES | |
| | APGLSDEKLNLTIQDDAYIPKYDSNGTSDIEQYDVSELNVFFY | |
| | LDAQKVPEGENNVDFTSSIDTALLEQPKIYTFFSSKFISNLNKT | |
| | MQAALFVSWIQQVLVDFTTEATQKSTVDKIADISIVVPYIGLAL | |
| | NIGNEAQKGNEKDALELLGAGILLEFEPELLIPIILVFTIKSFLGS | |
| | SDNKNKVIKAINNALKERDENWKEVYSFIVSNWMTKINTQFN | |
| | KRKEQMYQALQNQVNAIKTIIESKYNSYTLEEKNELKNKYDIE | |
| | QIENELNQTVSIAMNNIEIFLTESSISYLMKLINEVKINKLKEYD | |
| | ENVKTYLLDYIIKHGSILGESQQELNSMVIDTLNNSIPEKLSSY | |
| | TDDKILISYENKFFKRIKSSSVLNMRYKNDKYVDTSGYDSNIN | |
| | IGSKVNKYPTNKNQFQLFNLESSEVNISQNDYIIYDNKYKNF | |
| | SISFWVRIPKYFNSISLNNEYTIINCMRDNNSGWKVSLNYGEI | |
| | IWTLQDNAGINQKLAFNYSQMINISDYINKWIFVTITNDRLGD | |
| | SKLYINGNLIDQKSILNLGNIHVSDNILFKIVNCRDTHRYIGIRY | |
| | FNIFDKELDETEIQTLYSNEPNTNILKDFWGNYLLYDKEYYLL | |
| | NVLKPNNYIDGRKDSTLSIKGPRGSVMTTNIYLNSSLYRGIK | |
| | VKIQRVNNSSTNDNLVRKNDQVYINFVASKTGLGPLYADTAT | |
| | TNKEKTIKISSSGNRFNQVVVMNSVGNNCTMNFKNNNGNNI | |
| | GLLGFKADTVVASTWYYTHMRDHTNSNGCFWNFISEDDGW | |
| | GERPL | |
| BoNT/E12 | MPKINSFNYNDPVNDRTILYIKPGGCQQFYKSFNIMKNIWIIPE | 15 |
| | RNVIGTIPQDFQPPTSLKNGDSSYYDPNYLQSNEEKDRFLKI | |
| | VTKIFNRINDNLSGGILLEELSKANPYLGNDNTPDGDFIINDAS | |
| | AVPIQFSNGSQSILLPNVIIMGAEPDLFETNSSNISLINNYRPS | |
| | NHGFGSIAIVTFSPEYSFRFNDNSMNEFIQDPALTLMHELIHS | |
| | LHGLYGAKGITTKYTITQQQNSLITNIRGINIEEFLTFGGNDLNII | |
| | TSSQFNDIYTNLLDDYKKIASKLSQVRVSNPQLNPYKDVFQE | |
| | KYGLDKDASGIYSVNINKFNDIFKKLYSFTEFDLATKFQVKCR | |
| | ETYIGQYKYFQLSNLLNDSIYNISEGYNINNLKVNFRGQNANL | |
| | NPRIITPITGRGLVKKIIRFCKNIVSVKGIRKSICIEVNNGELFFV | |
| | ASENSYNDDNINTPKEIDDTVTSNNNYENDLDQVILNFNSES | |
| | APGLSDEKLNLTIQDDAYIPKYDSNGTSDIEQHDVNELNVFFY | |
| | LDAQKVPEGENNVNLTSSIDTALLEQPKIYTFFSSEFINNVNK | |
| | PVQAVLFVSWIQQVLVDFTTEATQKSTVDKIADISIVVPYIGLA | |
| | LNIGNEAQKGNFKDALELLGAGILLEFVPELLIPTILVFTIKSFL | |
| | GSSDNKNKIIKAINNALKERDEKWKEVYSFIVSNWITKINTQF | |
| | NKRKEQMYQALQNQVNAIKTIIESKYNSYTLEEKNELTNKYDI | |
| | KQIENELNQKVSIAMNNIDRFLTESSISYLMKLINEVKINKLRE | |
| | YDENVKTYLLNYIIQHGSTLGESQQELNSMVINTLNNSIPFKL | |
| | SSYTDDKILISYFNKFFKRIKSSSVLNMRYKNDKYVDTSGYD | |
| | SNINIGSKVNKYPTNKNQFQLFNLESSEVNISQNDYIIYDNKY | |
| | KNFSISFWVRIPKYFNSISLNNEYTIINCMRDNNSGWKVSLN | |
| | YGEIIWTLQDNAGINQKLAFNYSQMINISDYINKWIFVTITNDR | |
| | LGDSKLYINGNLIDQKSILNLGNIHVSDNILFKIVNCRDTHRYI | |
| | GIRYFNIFDKELDETEIQTLYSNEPNTNILKDFWGNYLLYDKE | |
| | YYLLNVLKPNNYIDGRKDSTLSIKGPRGSVMTTNIYLNSSLY | |
| | RGIKVKIQRVNNSSTNDNLVRKNDQVYINFVASKTGLGPLYA | |
| | DTATTNKEKTIKISSSGNRFNQVVVMNSVGNNCTMNFKNNN | |
| | GNNIGLLGFKADTVVASTWYYTHMRDHTNSNGCFWNFISE | |
| | DDGWGERPL | |

In some other embodiments, SEQ ID NO: 3 (e.g., H_{C}E-V3.5) as described herein may be used to replace the H_{C} of any of the BoNT serotypes described herein (e.g., BoNT/A1 (SEQ ID NO: 16), BoNT/B1 (SEQ ID NO: 16), BoNT/C (SEQ ID NO: 17), BoNT/CD (SEQ ID NO: 18), BoNT/D (SEQ ID NO: 19), BoNT/DC (SEQ ID NO: 20), BoNT/F1 (SEQ ID NO: 21), BoNT/G (SEQ ID NO: 22), BoNT/HA (SEQ ID NO: 23), BoNT/Wo (SEQ ID NO: 24), BoNT/X (SEQ ID NO: 25), and BoNT/En (SEQ ID NO: 26)). Without wishing to limit the present invention to any particular theory or mechanism, it is believed that replacing the H_{C} domain of any of the aforementioned serotypes may give these toxins the unique receptor-binding features of H_{C}E-V3.5.

Table 3 shows representative sequences for chimeric designs of other BoNT serotypes with their endogenous receptor-binding domain replaced with H_{C}E-V3.5. The underlined sequences are that of the wild-type LC-H_{N} fragment of the indicated BoNT serotype, and the bolded sequence is H_{C}E-V3.5. As an example, the undermentioned sequences include only one representative subtype from each BoNT serotype. The sequences included in Table 3 are not the only designs the present invention encompasses. For example, the region that connects the LC-H_{N} fragment of the indicated BoNT serotype and H_{C}E-V3.5 adopts a flexible structure and thus, the amino acid compositions in this region can be varied without affecting its activity.

TABLE 3

| | | SEQ ID |
|---|---|---|
| | Sequence | NO: |
| BoNT/A1 | MPFVNKQFNYKDPVNGVDIAYIKIPNAGQMQPVKAFKIHNKIWVIP | 16 |
| | ERDTFTNPEEGDLNPPPEAKQVPVSYYDSTYLSTDNEKDNYLKG | |
| | VTKLFERIYSTDLGRMLLTSIVRGIPFWGGSTIDTELKVIDTNCINVI | |
| | QPDGSYRSEELNLVIIGPSADIIQFECKSFGHEVLNLTRNGYGSTQ | |
| | YIRFSPDFTFGFEESLEVDTNPLLGAGKFATDPAVTLAHELIHAGH | |
| | RLYGIAINPNRVFKVNTNAYYEMSGLEVSFEELRTFGGHDAKFID | |
| | SLQENEFRLYYYNKEKDIASTLNKAKSIVGTTASLQYMKNVEKEK | |
| | YLLSEDTSGKFSVDKLKFDKLYKMLTEIYTEDNFVKFFKVLNRKTY | |
| | LNEDKAVEKINIVPKVNYTIYDGFNLRNTNLAANENGQNTEINNMN | |
| | FTKLKNFTGLFEFYKLLCVRGIITSKTKSLDKGYNKALNDLCIKVN | |
| | NWDLFFSPSEDNFTNDLNKGEEITSDTNIEAAEENISLDLIQQYYL | |
| | TENEDNEPENISIENLSSDIIGQLELMPNIEREPNGKKYELDKYTM | |
| | FHYLRAQEFEHGKSRIALTNSVNEALLNPSRVYTFFSSDYVKKVN | |
| | KATEAAMFLGWVEQLVYDFTDETSEVSTTDKIADITIIIPYIGPALNI | |
| | GNMLYKDDFVGALIFSGAVILLEFIPEIAIPVLGTFALVSYIANKVLTV | |
| | QTIDNALSKRNEKWDEVYKYIVTNWLAKVNTQIDLIRKKMKEALE | |
| | NQAEATKAIINYQYNQYTEEEKNNINFNIDDLSSKLNESINKAMINI | |
| | NKFLNQCSVSYLMNSMIPYGVKRLEDEDASLKDALLKYIYDNRGT | |
| | LIGQVDRLKDKVNNTLSTDIPFQLSKYVDNQRLLSTFTEYIKRIKS | |
| | SSVLNMRYKNDKYVDTSGYDSNINIGSKVNKYPTNKNQFQLFNL | |
| | ESSEVNISQNDYIIYDNKYKNFSISFWVRIPKYFNSISLNNEYTIIN | |
| | CMRDNNSGWKVSLNYGEIIWTLQDNAGINQKLAFNYSQMINISD | |
| | YINKWIFVTITNDRLGDSKLYINGNLIDQKSILNLGNIHVSDNILFKI | |
| | VNCRDTHRYIGIRYFNIFDKELDETEIQTLYSNEPNTNILKDFWGN | |
| | YLLYDKEYYLLNVLKPNNYIDGRKDSTLSIKGPRGSVMTTNIYLN | |
| | SSLYRGIKVKIQRVNNSSTNDNLVRKNDQVYINFVASKTGLGPLY | |
| | ADTATTNKEKTIKISSSGNRFNQVVVMNSVGNNCTMNFKNNNG | |
| | NNIGLLGFKADTVVASTWYYTHMRDHTNSNGCFWNFISEDDGW | |
| | GERPL | |
| BoNT/B1 | MPVTINNENYNDPIDNNNIIMMEPPFARGTGRYYKAFKITDRIWIIP | 17 |
| | ERYTFGYKPEDFNKSSGIFNRDVCEYYDPDYLNTNDKKNIFLQT | |
| | MIKLFNRIKSKPLGEKLLEMIINGIPYLGDRRVPLEEFNTNIASVTV | |
| | NKLISNPGEVERKKGIFANLIIFGPGPVLNENETIDIGIQNHFASRE | |
| | GFGGIMQMKFCPEYVSVFNNVQENKGASIFNRRGYFSDPALILM | |
| | HELIHVLHGLYGIKVDDLPIVPNEKKFFMQSTDAIQAEELYTFGGQ | |
| | DPSIITPSTDKSIYDKVLQNFRGIVDRLNKVLVCISDPNININIYKNK | |
| | FKDKYKFVEDSEGKYSIDVESFDKLYKSLMFGFTETNIAENYKIKT | |
| | RASYFSDSLPPVKIKNLLDNEIYTIEEGFNISDKDMEKEYRGQNKA | |
| | INKQAYEEISKEHLAVYKIQMCKSVKAPGICIDVDNEDLFFIADKNS | |
| | FSDDLSKNERIEYNTQSNYIENDFPINELILDTDLISKIELPSENTES | |
| | LTDFNVDVPVYEKQPAIKKIFTDENTIFQYLYSQTFPLDIRDISLTSS | |
| | FDDALLESNKVYSFFSMDYIKTANKVVEAGLFAGWVKQIVNDEVI | |
| | EANKSNTMDKIADISLIVPYIGLALNVGNETAKGNFENAFEIAGASI | |
| | LLEFIPELLIPVVGAFLLESYIDNKNKIIKTIDNALTKRNEKWSDMYG | |
| | LIVAQWLSTVNTQFYTIKEGMYKALNYQAQALEEIIKYRYNIYSEK | |
| | EKSNINIDFNDINSKLNEGINQAIDNINNFINGCSVSYLMKKMIPLA | |
| | VEKLLDFDNTLKKNLLNYIDENKLYLIGSAEYEKSKVNKYLKTIMPF | |
| | DLSIYTNDTILIEMFNKYNSRIKSSSVLNMRYKNDKYVDTSGYDS | |
| | NINIGSKVNKYPTNKNQFQLFNLESSEVNISQNDYIIYDNKYKNF | |
| | SISFWVRIPKYFNSISLNNEYTIINCMRDNNSGWKVSLNYGEIIWT | |
| | LQDNAGINQKLAFNYSQMINISDYINKWIFVTITNDRLGDSKLYIN | |
| | GNLIDQKSILNLGNIHVSDNILFKIVNCRDTHRYIGIRYFNIFDKEL | |
| | DETEIQTLYSNEPNTNILKDFWGNYLLYDKEYYLLNVLKPNNYID | |
| | GRKDSTLSIKGPRGSVMTTNIYLNSSLYRGIKVKIQRVNNSSTND | |
| | NLVRKNDQVYINFVASKTGLGPLYADTATTNKEKTIKISSSGNRF | |
| | NQVVVMNSVGNNCTMNFKNNNGNNIGLLGFKADTVVASTWYY | |
| | THMRDHTNSNGCFWNFISEDDGWGERPL | |
| BoNT/C | MPITINNFNYSDPVDNKNILYLDTHLNTLANEPEKAFRITGNIWVIP | 18 |
| | DRFSRNSNPNLNKPPRVTSPKSGYYDPNYLSTDSDKDTFLKEIIK | |
| | LFKRINSREIGEELIYRLSTDIPFPGNNNTPINTFDFDVDFNSVDVK | |
| | TRQGNNWVKTGSINPSVIITGPRENIIDPETSTEKLTNNTFAAQEG | |
| | FGALSIISISPRFMLTYSNATNDVGEGRFSKSEFCMDPILILMHELN | |
| | HAMHNLYGIAIPNDQTISSVTSNIFYSQYNVKLEYAEIYAFGGPTID | |
| | LIPKSARKYFEEKALDYYRSIAKRLNSITTANPSSFNKYIGEYKQKL | |
| | IRKYRFVVESSGEVTVNRNKFVELYNELTQIFTEFNYAKIYNVQNR | |
| | KIYLSNVYTPVTANILDDNVYDIQNGFNIPKSNLNVLFMGQNLSRN | |
| | PALRKVNPENMLYLFTKFCHKAIDGRSLYNKTLDCRELLVKNTDL | |
| | PFIGDISDVKTDIFLRKDINEETEVIYYPDNVSVDQVILSKNTSEHG | |
| | QLDLLYPSIDSESEILPGENQVFYDNRTQNVDYLNSYYYLESQKL | |
| | SDNVEDFTFTRSIEEALDNSAKVYTYFPTLANKVNAGVQGGLFL | |
| | MWANDVVEDFTTNILRKDTLDKISDVSAIIPYIGPALNISNSVRRGN | |
| | FTEAFAVTGVTILLEAFPEFTIPALGAFVIYSKVQERNEIIKTIDNCLE | |
| | QRIKRWKDSYEWMMGTWLSRIITQFNNISYQMYDSLNYQAGAIK | |
| | AKIDLEYKKYSGSDKENIKSQVENLKNSLDVKISEAMNNINKFIRE | |
| | CSVTYLFKNMLPKVIDELNEFDRNTKAKLINLIDSHNIILVGEVDKL | |
| | KAKVNNSFQNTIPFNIFSYTNNSLLKDIINEYFNRIKSSSVLNMRY | |
| | KNDKYVDTSGYDSNINIGSKVNKYPTNKNQFQLFNLESSEVNIS | |
| | QNDYIIYDNKYKNFSISFWVRIPKYFNSISLNNEYTIINCMRDNNS | |
| | GWKVSLNYGEIIWTLQDNAGINQKLAFNYSQMINISDYINKWIFV | |
| | TITNDRLGDSKLYINGNLIDQKSILNLGNIHVSDNILFKIVNCRDTH | |
| | RYIGIRYFNIFDKELDETEIQTLYSNEPNTNILKDFWGNYLLYDKE | |
| | YYLLNVLKPNNYIDGRKDSTLSIKGPRGSVMTTNIYLNSSLYRGI | |
| | KVKIQRVNNSSTNDNLVRKNDQVYINFVASKTGLGPLYADTATTN | |
| | KEKTIKISSSGNRFNQVVVMNSVGNNCTMNFKNNNGNNIGLLGF | |
| | KADTVVASTWYYTHMRDHTNSNGCFWNFISEDDGWGERPL | |
| BoNT/CD | MPITINNENYSDPVDNKNILYLDTHLNTLANEPEKAFRIIGNIWVIP | 19 |
| | DRFSRDSNPNLNKPPRVTSPKSGYYDPNYLSTDSEKDTFLKEIIK | |
| | LFKRINSREIGEELIYRLATDIPFPGNNNTPINTFDFDVDFNSVDVK | |
| | TRQGNNWVKTGSINPSVIITGPRENIIDPETSTFKLTNNTFAAQEG | |
| | FGALSIISISPRFMLTYSNATNNVGEGRFSKSEFCMDPILILMHELN | |
| | HAMHNLYGIAIPNDQRISSVTSNIFYSQYKVKLEYAEIYAEGGPTID | |
| | LIPKSARKYFEEKALDYYRSIAKRLNSITTANPSSFNKYIGEYKQKL | |
| | IRKYRFVVESSGEVAVDRNKFAELYKELTQIFTEFNYAKIYNVQNR | |
| | KIYLSNVYTPVTANILDDNVYDIQNGFNIPKSNLNVLFMGQNLSRN | |
| | PALRKVNPENMLYLFTKFCHKAIDGRSLYNKTLDCRELLVKNTDL | |
| | PFIGDISDIKTDIFLSKDINEETEVIDYPDNVSVDQVILSKNTSEHG | |
| | QLDLLYPIIEGESQVLPGENQVFYDNRTQNVDYLNSYYYLESQKL | |
| | SDNVEDFTFTTSIEEALDNSGKVYTYFPKLADKVNTGVQGGLFL | |
| | MWANDVVEDFTTNILRKDTLDKISDVSAIIPYIGPALNISNSVRRGN | |
| | FTEAFAVTGVTILLEAFQEFTIPALGAFVIYSKVQERNEIIKTIDNCL | |
| | EQRIKRWKDSYEWMIGTWLSRITTQFNNISYQMYDSLNYQADAI | |
| | KDKIDLEYKKYSGSDKENIKSQVENLKNSLDIKISEAMNNINKFIRE | |
| | CSVTYLFKNMLPKVIDELNKFDLKTKTELINLIDSHNIILVGEVDRLK | |
| | AKVNESFENTIPFNIFSYTNNSLLKDIINEYFNRIKSSSVLNMRYKN | |
| | DKYVDTSGYDSNINIGSKVNKYPTNKNQFQLFNLESSEVNISQN | |
| | DYIIYDNKYKNFSISFWVRIPKYFNSISLNNEYTIINCMRDNNSGW | |
| | KVSLNYGEIIWTLQDNAGINQKLAFNYSQMINISDYINKWIFVTIT | |
| | NDRLGDSKLYINGNLIDQKSILNLGNIHVSDNILFKIVNCRDTHRYI | |
| | GIRYFNIFDKELDETEIQTLYSNEPNTNILKDFWGNYLLYDKEYYL | |
| | LNVLKPNNYIDGRKDSTLSIKGPRGSVMTTNIYLNSSLYRGIKVKI | |
| | QRVNNSSTNDNLVRKNDQVYINFVASKTGLGPLYADTATTNKEK | |
| | TIKISSSGNRFNQVVVMNSVGNNCTMNFKNNNGNNIGLLGFKA | |
| | DTVVASTWYYTHMRDHTNSNGCFWNFISEDDGWGERPL | |
| BoNT/D | MTWPVKDFNYSDPVNDNDILYLRIPQNKLITTPVKAFMITQNIWVI | 20 |
| | PERFSSDTNPSLSKPPRPTSKYQSYYDPSYLSTDEQKDTFLKGII | |
| | KLFKRINERDIGKKLINYLVVGSPFMGDSSTPEDTFDFTRHTTNIA | |
| | VEKFENGSWKVTNIITPSVLIFGPLPNILDYTASLTLQGQQSNPSF | |
| | EGFGTLSILKVAPEFLLTESDVTSNQSSAVLGKSIFCMDPVIALMH | |
| | ELTHSLHQLYGINIPSDKRIRPQVSEGFFSQDGPNVQFEELYTFG | |
| | GLDVEIIPQIERSQLREKALGHYKDIAKRLNNINKTIPSSWISNIDKY | |
| | KKIFSEKYNFDKDNTGNFVVNIDKENSLYSDLTNVMSEVVYSSQY | |
| | NVKNRTHYFSRHYLPVFANILDDNIYTIRDGFNLTNKGFNIENSGQ | |
| | NIERNPALQKLSSESVVDLFTKVCLRLTKNSRDDSTCIKVKNNRL | |
| | PYVADKDSISQEIFENKIITDETNVQNYSDKFSLDESILDGQVPINP | |
| | EIVDPLLPNVNMEPLNLPGEEIVFYDDITKYVDYLNSYYYLESQKL | |
| | SNNVENITLTTSVEEALGYSNKIYTELPSLAEKVNKGVQAGLELN | |
| | WANEVVEDFTTNIMKKDTLDKISDVSVIIPYIGPALNIGNSALRGNF | |
| | NQAFATAGVAFLLEGFPEFTIPALGVFTFYSSIQEREKIIKTIENCLE | |
| | QRVKRWKDSYQWMVSNWLSRITTQFNHINYQMYDSLSYQADAI | |
| | KAKIDLEYKKYSGSDKENIKSQVENLKNSLDVKISEAMNNINKFIR | |
| | ECSVTYLFKNMLPKVIDELNKFDLRTKTELINLIDSHNIILVGEVDR | |
| | LKAKVNESFENTMPFNIFSYTNNSLLKDIINEYFNRIKSSSVLNMR | |
| | YKNDKYVDTSGYDSNINIGSKVNKYPTNKNQFQLFNLESSEVNI | |
| | SQNDYIIYDNKYKNFSISFWVRIPKYFNSISLNNEYTIINCMRDNN | |
| | SGWKVSLNYGEIIWTLQDNAGINQKLAFNYSQMINISDYINKWIF | |
| | VTITNDRLGDSKLYINGNLIDQKSILNLGNIHVSDNILFKIVNCRDT | |
| | HRYIGIRYFNIFDKELDETEIQTLYSNEPNTNILKDFWGNYLLYDK | |
| | EYYLLNVLKPNNYIDGRKDSTLSIKGPRGSVMTTNIYLNSSLYRG | |
| | IKVKIQRVNNSSTNDNLVRKNDQVYINFVASKTGLGPLYADTATT | |
| | NKEKTIKISSSGNRFNQVVVMNSVGNNCTMNFKNNNGNNIGLL | |
| | GFKADTVVASTWYYTHMRDHTNSNGCFWNFISEDDGWGERPL | |
| BoNT/DC | MTWPVKDENYSDPVNDNDILYLRIPQNKLITTPVKAFMITQNIWVI | |
| | PERFSSDINPSLSKPPRPTSKYQSYYDPSYLSTDEQKDTFLKGII | |
| | KLFKRINERDIGKKLINYLVVGSPFMGDSSTPEDTFDFTRHTTNIA | |
| | VEKFENGSWKVTNIITPSVLIEGPLPNILDYTASITLQGQQSNPSE | |
| | EGFGTLSILKVAPEFLLTFSDVTSNQSSAVLGKSIFCMDPVIALMH | |
| | ELTHSLHQLYGINIPSDKRIRPQVSEGFFSQDGPNVQFEELYTFG | |
| | GSDVEIIPQIERLQLREKALGHYKDIAKRLNNINKTIPSSWSSNIDK | |
| | YKKIFSEKYNEDKQNTGNFVVNIDKFNSLYSDLTNVMSEVVYSSQ | |
| | YNVKNRTHYFSKHYLPVFANILDDNIYTIINGFNLTTKGFNIENSGQ | |
| | NIERNPALQKLSSESVVDLFTKVCLRLTRNSRDDSTCIQVKNNTL | |
| | PYVADKDSISQEIFESQIITDETNVENYSDNFSLDESILDAKVPTNP | |
| | EAVDPLLPNVNMEPLNVPGEEEVFYDDITKDVDYLNSYYYLEAQ | |
| | KLSNNVENITLTTSVEEALGYSNKIYTELPSLAEKVNKGVQAGLFL | |
| | NWANEVVEDFTTNIMKKDTLDKISDVSAIIPYIGPALNIGNSALRGN | |
| | FKQAFATAGVAFLLEGFPEFTIPALGVFTFYSSIQEREKIIKTIENCL | |
| | EQRVKRWKDSYQWMVSNWLSRITTQFNHISYQMYDSLSYQADA | |
| | IKAKIDLEYKKYSGSDKENIKSQVENLKNSLDVKISEAMNNINKFIR | |
| | ECSVTYLFKNMLPKVIDELNKFDLKTKTELINLIDSHNIILVGEVDRL | |
| | KAKVNESFENTIPFNIFSYTNNSLLKDMINEYFNRIKSSSVLNMRY | |
| | KNDKYVDTSGYDSNINIGSKVNKYPTNKNQFQLFNLESSEVNIS | |
| | QNDYIIYDNKYKNFSISFWVRIPKYFNSISLNNEYTIINCMRDNNS | |
| | GWKVSLNYGEIIWTLQDNAGINQKLAFNYSQMINISDYINKWIFV | |
| | TITNDRLGDSKLYINGNLIDQKSILNLGNIHVSDNILFKIVNQRDTH | |
| | RYIGIRYFNIFDKELDETEIQTLYSNEPNTNILKDFWGNYLLYDKE | |
| | YYLLNVLKPNNYIDGRKDSTLSIKGPRGSVMTTNIYLNSSLYRGI | |
| | KVKIQRVNNSSTNDNLVRKNDQVYINFVASKTGLGPLYADTATTN | |
| | KEKTIKISSSGNRFNQVVVMNSVGNNCTMNFKNNNGNNIGLLGF | |
| | KADTVVASTWYYTHMRDHTNSNGCFWNFISEDDGWGERPL | |
| BoNT/F1 | MPVVINSFNYNDPVNDDTILYMQIPYEEKSKKYYKAFEIMRNVWII | 21 |
| | PERNTIGIDPSDEDPPASLENGSSAYYDPNYLTTDAEKDRYLKTT | |
| | IKLFKRINSNPAGEVLLQEISYAKPYLGNEHTPINEFHPVTRTTSVN | |
| | IKSSTNVKSSIILNLLVLGAGPDIFENSSYPVRKLMDSGGVYDPSN | |
| | DGEGSINIVTESPEYEYTENDISGGYNSSTESFIADPAISLAHELIH | |
| | ALHGLYGARGVTYKETIKVKQAPLMIAEKPIRLEEFLTFGGQDLNII | |
| | TSAMKEKIYNNLLANYEKIATRLSRVNSAPPEYDINEYKDYFQWK | |
| | YGLDKNADGSYTVNENKENEIYKKLYSETEIDLANKEKVKQRNTY | |
| | FIKYGFLKVPNLLDDDIYTVSEGENIGNLAVNNRGQNIKLNPKIIDSI | |
| | PDKGLVEKIVKFCKSVIPRKGTKAPPRLCIRVNNRELFFVASESSY | |
| | NENDINTPKEIDDTTNLNNNYRNNLDEVILDYNSETIPQISNQTLN | |
| | TLVQDDSYVPRYDSNGTSEIEEHNVVDLNVFFYLHAQKVPEGET | |
| | NISLTSSIDTALSEESQVYTFFSSEFINTINKPVHAALFISWINQVIR | |
| | DFTTEATQKSTFDKIADISLVVPYVGLALNIGNEVQKENFKEAFEL | |
| | LGAGILLEFVPELLIPTILVFTIKSFIGSSENKNKIIKAINNSLMERET | |
| | KWKEIYSWIVSNWLTRINTQFNKRKEQMYQALQNQVDAIKTVIEY | |
| | KYNNYTSDERNRLESEYNINNIREELNKKVSLAMENIERFITESSIE | |
| | YLMKLINEAKVSKLREYDEGVKEYLLDYISEHRSILGNSVQELNDL | |
| | VTSTLNNSIPFELSSYTNDKILILYFNKLYKRIKSSSVLNMRYKNDK | |
| | YVDTSGYDSNINIGSKVNKYPTNKNQFQLFNLESSEVNISQNDYII | |
| | YDNKYKNFSISFWVRIPKYFNSISLNNEYTIINCMRDNNSGWKVS | |
| | LNYGEIIWTLQDNAGINQKLAFNYSQMINISDYINKWIFVTITNDR | |
| | LGDSKLYINGNLIDQKSILNLGNIHVSDNILFKIVNCRDTHRYIGIR | |
| | YFNIFDKELDETEIQTLYSNEPNTNILKDFWGNYLLYDKEYYLLN | |
| | VLKPNNYIDGRKDSTLSIKGPRGSVMTTNIYLNSSLYRGIKVKIQ | |
| | RVNNSSTNDNLVRKNDQVYINFVASKTGLGPLYADTATTNKEKTI | |
| | KISSSGNRFNQVVVMNSVGNNCTMNFKNNNGNNIGLLGFKADT | |
| | VVASTWYYTHMRDHTNSNGCFWNFISEDDGWGERPL | |
| BoNT/G | MPVNIKNFNYNDPINNDDIIMMEPENDPGPGTYYKAFRIIDRIWIV | 22 |
| | PERFTYGFQPDQFNASTGVFSKDVYEYYDPTYLKTDAEKDKFLK | |
| | TMIKLFNRINSKPSGQRLLDMIVDAIPYLGNASTPPDKFAANVANV | |
| | SINKKIIQPGAEDQIKGLMTNLIIFGPGPVLSDNFTDSMIMNGHSPI | |
| | SEGFGARMMIRFCPSCLNVFNNVQENKDTSIFSRRAYFADPALTL | |
| | MHELIHVLHGLYGIKISNLPITPNTKEFFMQHSDPVQAEELYTEGG | |
| | HDPSVISPSTDMNIYNKALQNFQDIANRLNIVSSAQGSGIDISLYK | |
| | QIYKNKYDFVEDPNGKYSVDKDKFDKLYKALMFGFTETNLAGEY | |
| | GIKTRYSYESEYLPPIKTEKLLDNTIYTQNEGFNIASKNLKTEFNG | |
| | QNKAVNKEAYEEISLEHLVIYRIAMCKPVMYKNTGKSEQCIVNNE | |
| | DLFFIANKDSFSKDLAKAETIAYNTQNNTIENNFSIDQLILDNDLSS | |
| | GIDLPNENTEPFTNFDDIDIPVYIKQSALKKIFVDGDSLFEYLHAQT | |
| | FPSNIENLQLTNSLNDALRNNNKVYTFFSTNLVEKANTVVGASLF | |
| | VNWVKGVIDDFTSESTQKSTIDKVSDVSIIIPYIGPALNVGNETAKE | |
| | NFKNAFEIGGAAILMEFIPELIVPIVGFFTLESYVGNKGHIIMTISNA | |
| | LKKRDQKWTDMYGLIVSQWLSTVNTQFYTIKERMYNALNNQSQ | |
| | AIFKIFDQYNRYSEEDKMNINIFNDIDFKLNQSINLAINNIDDFIN | |
| | QCSISYLMNRMIPLAVKKLKDFDDNLKRDLLEYIDTNELYLLDEVNI | |
| | LKSKVNRHLKDSIPFDLSLYTKDTILIQVFNNYISRIKSSSVLNMRY | |
| | KNDKYVDTSGYDSNINIGSKVNKYPTNKNQFQLFNLESSEVNIS | |
| | QNDYIIYDNKYKNFSISFWVRIPKYFNSISLNNEYTIINCMRDNNS | |
| | GWKVSLNYGEIIWTLQDNAGINQKLAFNYSQMINISDYINKWIFV | |
| | TITNDRLGDSKLYINGNLIDQKSILNLGNIHVSDNILFKIVNCRDTH | |
| | RYIGIRYFNIFDKELDETEIQTLYSNEPNTNILKDFWGNYLLYDKE | |
| | YYLLNVLKPNNYIDGRKDSTLSIKGPRGSVMTTNIYLNSSLYRGI | |
| | KVKIQRVNNSSTNDNLVRKNDQVYINFVASKTGLGPLYADTATTN | |
| | KEKTIKISSSGNRFNQVVVMNSVGNNCTMNFKNNNGNNIGLLGF | |
| | KADTVVASTWYYTHMRDHTNSNGCFWNFISEDDGWGERPL | |
| BoNT/HA | MPVVINSFNYDDPVNDNTIIYIRPPYYETSNTYEKAFQIMDNVWIIP | 23 |
| | ERYRLGIDPSLFNPPVSLKAGSDGYFDPNYLSTNTEKNKYLQIMI | |
| | KLFKRINSKPAGQILLEEIKNAIPYLGNSYTQEEQFTTNNRTVSFN | |
| | VKLANGNIVQQMANLIIWGPGPDLTTNKTGGIIYSPYQSMEATPY | |
| | KDGFGSIMTVEFSPEYATAFNDISIASHSPSLFIKDPALILMHELIHV | |
| | LHGLYGTYITEYKITPNVVQSYMKVTKPITSAEFLTFGGRDRNIVP | |
| | QSIQSQLYNKVLSDYKRIASRLNKVNTATALINIDEFKNLYEWKYQ | |
| | FAKDSNGVYSVDLNKFEQLYKKIYSFTEFNLAYEFKIKTRLGYLAE | |
| | NFGPFYLPNLLDDSIYTEVDGFNIGALSINYQGQNIGSDINSIKKLQ | |
| | GQGVVSRVVRLCSNSNTKNSLCITVNNRDLFFIASQESYGENTIN | |
| | TYKEIDDTTTLDPSFEDILDKVILNFNEQVIPQMPNRNVSTDIQKD | |
| | NYIPKYDYNRTDIIDSYEVGRNYNTFFYLNAQKFSPNESNITLTSS | |
| | FDTGLLEGSKVYTFFSSDFINNINKPVQALLFIEWVKQVIRDFTTE | |
| | ATKTSTVDKLKDISLVVPYIGLALNIGDEIYKQHFAEAVELVGAGLL | |
| | LEFSPEFLIPTLLIFTIKGYLTGSIRDKDKIIKTLDNALNVRDQKWKE | |
| | LYRWVVSKWLTTINTQFNKRKEQMYKALKNQATAIKKIIENKYNNY | |
| | TTDEKSKIDSSYNINEIERTLNEKINLAMKNIEQFITESSIAYLINIINN | |
| | ETIQKLKSYDDLVRRYLLGYIRNHSSILGNSVEELNSKVNNHLDN | |
| | GIPFELSSYTNDSLLIRYFNKNYGRIKSSSVLNMRYKNDKYVDTS | |
| | GYDSNINIGSKVNKYPTNKNQFQLFNLESSEVNISQNDYIIYDNK | |
| | YKNFSISFWVRIPKYFNSISLNNEYTIINCMRDNNSGWKVSLNYG | |
| | EIIWTLQDNAGINQKLAFNYSQMINISDYINKWIFVTITNDRLGDS | |
| | KLYINGNLIDQKSILNLGNIHVSDNILFKIVNCRDTHRYIGIRYFNIF | |
| | DKELDETEIQTLYSNEPNTNILKDFWGNYLLYDKEYYLLNVLKPN | |
| | NYIDGRKDSTLSIKGPRGSVMTTNIYLNSSLYRGIKVKIQRVNNS | |
| | STNDNLVRKNDQVYINFVASKTGLGPLYADTATTNKEKTIKISSS | |
| | GNRFNQVVVMNSVGNNCTMNFKNNNGNNIGLLGFKADTVVAS | |
| | TWYYTHMRDHTNSNGCFWNFISEDDGWGERPL | |
| BoNT/Wo | MDVLEMFDVNYESPILESFDSTTQSLNDVHVFMSRIQMSAYDAD | 24 |
| | GEGRIEYRNLKLYEISSGIFISTDRLDTGASGVEDDHEMVDYYSS | |
| | ARLTREFLGESLDSQKSDYFEGIKKVFSFYKNKCNESRYIKEFFE | |
| | EIQFRNICGFPKQAGTSSTDIFDQFNSVDVLLQDPVTSVWNKKV | |
| | GSKKANIVIIPPATNLPITEACATAGFQPEGFPKLGSGSFFTVQFD | |
| | PFFSTRFKAHETDDVALLDPTLTLLHEMTHGLHFQKGIANPVNRS | |
| | GETPAWATTWGRVTGDNDAFKETPMEELLTFNKHTIDDDIEISDH | |
| | LKSTYIGFLYNGRNEDDPTESVDGVYQNVSSFLNQYRGFEISSDF | |
| | QHFIESCYGVKYNQESKKFIVNPRNIKRYVQDGFFIDEAKFARILNI | |
| | KTRSYYTIMPDNLGVWSYRVDILNRLRETFDEDRGLLSQELDFH | |
| | TALTPVVSENPALELEVAGMQRMVSLPKIKASYLPSDIKIKNFTGQ | |
| | KISHDTILDTNISGIIISKIKYKSDFVVDESMPRSSLNTTNYNLSPIK | |
| | GTKFETDIRDKISVKVTVSEITAPMINHVMKLDNSKVLTERPSLNE | |
| | DLEETFKNTKDVYIPKTTAMMKLKEGADQTLGAVGFAVWSGQILE | |
| | DLYNLAQKKEVSIDQIKDDLMSILPFYCAYKNLSAEKYEQAFANAT | |
| | LDAELIFATDGGGFAGLIGITVGAIAINSMYAKAETMEAYDSMFGKY | |
| | VDQYQNDIKNFTLNAYVQWENNILSRLWNESRLAITGFRNMLKTV | |
| | KTVMEFDATNQAYSEEDRKIIKAKCEEIFSEFPMLMQTFAKNSMT | |
| | ANLENASKIFNDIVWQKIKEELDQYVIDSKKYFLDSLEEAYNNGSI | |
| | SAESYYKYQTEAREKFVSPREVIDLYIAAHDTVVKRKRYIRRYSR | |
| | RIKSSSVLNMRYKNDKYVDTSGYDSNINIGSKVNKYPTNKNQFQ | |
| | LFNLESSEVNISQNDYIIYDNKYKNFSISFWVRIPKYFNSISLNNE | |
| | YTIINCMRDNNSGWKVSLNYGEIIWTLQDNAGINQKLAFNYSQM | |
| | NISDYINKWIFVTITNDRLGDSKLYINGNLIDQKSILNLGNIHVSDNI | |
| | LFKIVNCRDTHRYIGIRYFNIFDKELDETEIQTLYSNEPNTNILKDF | |
| | WGNYLLYDKEYYLLNVLKPNNYIDGRKDSTLSIKGPRGSVMTTN | |
| | IYLNSSLYRGIKVKIQRVNNSSTNDNLVRKNDQVYINFVASKTGL | |
| | GPLYADTATTNKEKTIKISSSGNRFNQVVVMNSVGNNCTMNFKN | |
| | NNGNNIGLLGFKADTVVASTWYYTHMRDHTNSNGCFWNFISED | |
| | DGWGERPL | |
| BoNT/X | MKLEINKFNYNDPIDGINVITMRPPRHSDKINKGKGPEKAFQVIKNI | 25 |
| | WIVPERYNFTNNTNDLNIPSEPIMEADAIYNPNYLNTPSEKDEFLQ | |
| | GVIKVLERIKSKPEGEKLLELISSSIPLPLVSNGALTLSDNETIAYQE | |
| | NNNIVSNLQANLVIYGPGPDIANNATYGLYSTPISNGEGTLSEVSF | |
| | SPFYLKPFDESYGNYRSLVNIVNKFVKREFAPDPASTLMHELVHV | |
| | THNLYGISNRNFYYNFDTGKIETSRQQNSLIFEELLTFGGIDSKAIS | |
| | SLIIKKIIETAKNNYTTLISERLNTVTVENDLLKYIKNKIPVQGRLGNF | |
| | KLDTAEFEKKLNTILFVLNESNLAQRFSILVRKHYLKERPIDPIYVNI | |
| | LDDNSYSTLEGFNISSQGSNDFQGQLLESSYFEKIESNALRAFIKI | |
| | CPRNGLLYNAIYRNSKNYLNNIDLEDKKTTSKTNVSYPCSLLNGC | |
| | EVENKDLFLISNKDSLNDINLSEEKIKPETTVFFKDKLPPQDITLSN | |
| | YDFTEANSIPSISQQNILERNEELYEPIRNSLFEIKTIYVDKLTTFHF | |
| | LEAQNIDESIDSSKIRVELTDSVDEALSNPNKVYSPFKNMSNTINSI | |
| | ETGITSTYIFYQWLRSIVKDFSDETGKIDVIDKSSDTLAIVPYIGPLL | |
| | NIGNDIRHGDFVGAIELAGITALLEYVPEFTIPILVGLEVIGGELARE | |
| | QVEAIVNNALDKRDQKWAEVYNITKAQWWGTIHLQINTRLAHTY | |
| | KALSRQANAIKMNMEFQLANYKGNIDDKAKIKNAISETEILLNKSV | |
| | EQAMKNTEKFMIKLSNSYLTKEMIPKVQDNLKNFDLETKKTLDKFI | |
| | KEKEDILGTNLSSSLRRKVSIRLNKNIAFDINDIPFSEFDDLINQYK | |
| | NRIKSSSVLNMRYKNDKYVDTSGYDSNINIGSKVNKYPTNKNQF | |
| | QLFNLESSEVNISQNDYIIYDNKYKNFSISFWVRIPKYFNSISLNN | |
| | EYTIINCMRDNNSGWKVSLNYGEIIWTLQDNAGINQKLAFNYSQ | |
| | MINISDYINKWIFVTITNDRLGDSKLYINGNLIDQKSILNLGNIHVS | |
| | DNILFKIVNQRDTHRYIGIRYFNIFDKELDETEIQTLYSNEPNTNILK | |
| | DFWGNYLLYDKEYYLLNVLKPNNYIDGRKDSTLSIKGPRGSVMT | |
| | TNIYLNSSLYRGIKVKIQRVNNSSTNDNLVRKNDQVYINFVASKT | |
| | GLGPLYADTATTNKEKTIKISSSGNRFNQVVVMNSVGNNCTMNF | |
| | KNNNGNNIGLLGFKADTVVASTWYYTHMRDHTNSNGCFWNFIS | |
| | EDDGWGERPL | |
| BoNT/En | MVTINDLHYSDPIDEDNIINMRIPLYDLEVDDQFINHNVPDLKAFQ | 26 |
| | VFPNVWVVPERYTFYSTMKNLDAPANPSRSSYYDPTYLQSDAEK | |
| | EVFLQQMILLFKRINSTQEGQQFLNLLSRSIPVPYESNGDVAMGT | |
| | TQVIKQMDDKGNVLKHRRAHIIIYGPGPDLMAKGSKALTKSRETG | |
| | RGCMAEIYFSPMYHKTYSTKLINKNSLVDKSVQEFVPDPAVTLIH | |
| | ELCHGIHALYGIDIGNVGSWEFNSNPNSLFSSWFSSKEAVNFEE | |
| | VMTFGGEDVKVIKSEIDKKIPGILNLIKTTVEPIINKITDPHDEMLQC | |
| | LQSKYPSLKGTLGQFFFDDTQLEKDIRDLWMVMNETMFAENLKA | |
| | LTRARYLVPKVENIVQVDILSPNVYTIDKGFNHLSKGFKGQSVSQ | |
| | SYFRKISALARGAVVRACPNPHFSSQRGLSSCIEILEDDLFIMSSK | |
| | DSFTDTDFSEPSVGPVSYKAKKGADTILDSTLSNYDFSKEINFTS | |
| | TVPIITVEDPLETDEDVPVISEDRTVYVDDYTTFHFLEAQKIGKEV | |
| | VPTQTKVVFTTNMEEALFDSKKVYTVFENTASRINEAGTGIANGM | |
| | MFYQWLKGIVQDFTEEATQKDTFDKISDVTMIVPYLGNILNIGNDI | |
| | RKGDFMGAVELGGVTILLEAIPELTLPVLIGLTIIEDELEKEQVSQT | |
| | VYNVLDKRDEKWEEVYGFVKQQWWWMVHTQFETRILHAYQAL | |
| | NHQVEAIKANMTYQLANYRGNQEDKELLEKAIDDTLQSLYYAVDQ | |
| | AMHNIKRFLIQSSKSYLLNQMLPKTKEQLLAFDQQTLRNVNDFIN | |
| | KNQGVLGESLAKDLKKKVEKRLTSLPVFNLEDLPISEFEDLIHSHE | |
| | IRIKSSSVLNMRYKNDKYVDTSGYDSNINIGSKVNKYPTNKNQF | |
| | QLFNLESSEVNISQNDYIIYDNKYKNFSISFWVRIPKYFNSISLNN | |
| | EYTIINCMRDNNSGWKVSLNYGEIIWTLQDNAGINQKLAFNYSQ | |
| | MINISDYINKWIFVTITNDRLGDSKLYINGNLIDQKSILNLGNIHVS | |
| | DNILFKIVNDRDTHRYIGIRYFNIFDKELDETEIQTLYSNEPNTNILK | |
| | DFWGNYLLYDKEYYLLNVLKPNNYIDGRKDSTLSIKGPRGSVMT | |
| | TNIYLNSSLYRGIKVKIQRVNNSSTNDNLVRKNDQVYINFVASKT | |
| | GLGPLYADTATTNKEKTIKISSSGNRFNQVVVMNSVGNNCTMNF | |
| | KNNNGNNIGLLGFKADTVVASTWYYTHMRDHTNSNGCFWNFIS | |
| | EDDGWGERPL | |

In some embodiments, the following BoNT/E residues can be modified to manipulate SV2 isoform selectivity: R1100, A1154, and F1160. These BoNT/E residues favorably interact with two key residues on SV2A (Y535 and Y557) and SV2B (Y478 and Y500), but are not compatible with the equivalent residues on SV2C (T521 and D543).

### Methods of Use:

The present invention may also feature a method for treating a therapeutic or cosmetic condition. For example, the method may comprise administering to a subject a composition comprising a modified serotype E botulinum neurotoxin (BoNT/E), e.g., a neurotoxin composition.

The compositions described herein may be administered using, for example, a needle or a needleless device. In certain embodiments, the method comprises subdermally injecting the composition into the subject. In some embodiments, the method comprises administering a composition comprising a modified serotype E botulinum neurotoxin (BoNT/E).

Injection of the compositions can be carried out by syringe, catheters, needles and other appropriate means. The injection can be performed on any area of the mammal's body that is in need of treatment, including, but not limited to, face, neck, torso, arms, hands, legs, and feet. The injection can be into any position in the specific area such as epidermis, dermis, fat, muscle, or subcutaneous layer.

In certain embodiments, the neurotoxin composition of the present invention can be used for cosmetic purposes such as to treat wrinkles, e.g., glabellar lines or canthal lines.

In certain embodiments, the neurotoxin composition of the present invention can be used for non-cosmetic purposes. Specifically, the compositions described herein can be used in the treatment of diverse diseases and disorders, including but not limited to chronic migraine, foot pain, idiopathic overactive bladder, detrusor overactivity associated with neurological conditions, upper limb spasticity, cervical dystonia, primary axillary hyperhidrosis, blepharospasm, strabismus, and similar ailments

However, the present invention should not be limited to only the diseases, disorders, or conditions disclosed herein. Rather, the invention encompasses the versatile application of the disclosed neurotoxin composition in treating any ailment where botulinum toxin has demonstrated utility.

### Example 1

The following is a non-limiting example of the present invention. It is to be understood that said example is not intended to limit the present invention in any way. Equivalents or substitutes are within the scope of the present invention.

At the molecular level, BoNT/E recognizes synaptic vesicle glycoprotein 2 (SV2), a family of 12-transmembrane domain proteins that also serve as receptors for BoNT/A, BoNT/D, BoNT/F, and the related tetanus neurotoxin. SV2 comprises three homologous isoforms, SV2A, 2B, and 2C, in mammals. Despite their similar primary sequences (~60% identity), only SV2A and SV2B, but not SV2C, are able to mediate the cell entry of BoNT/E into cultured hippocampal and cortical neurons, although it remains to be validated whether SV20 in motor neurons may still function as a receptor for BoNT/E. This is in sharp contrast to BoNT/A, which is able to use all three SV2 isoforms for cell entry. As the three SV2 isoforms have different tissue distributions in humans, the different specificities toward SV2 isoforms between BoNT/E and BoNT/A can contribute to potential differences in pharmacological and therapeutic features.

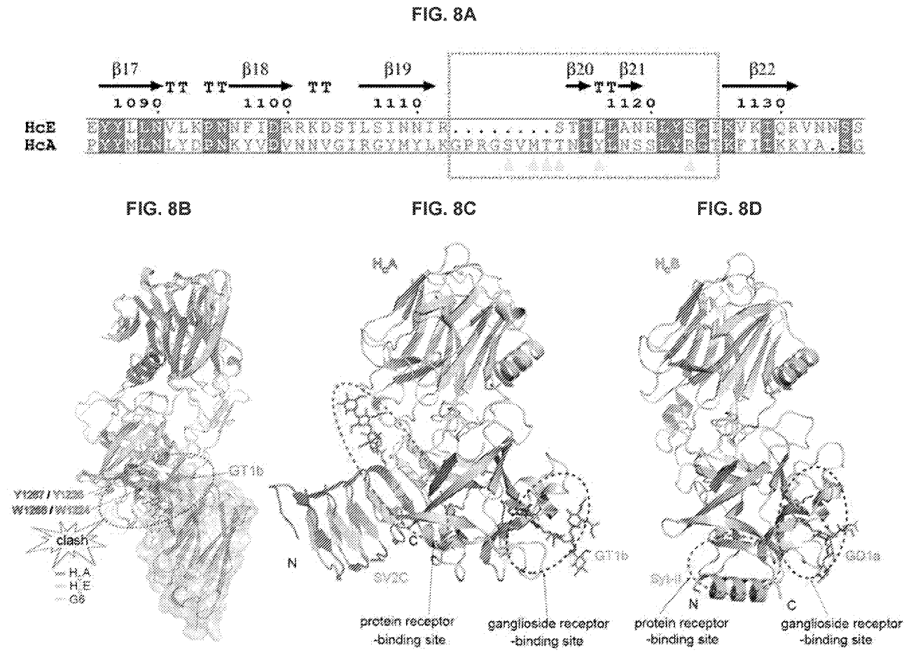

How BoNT/E manages to distinguish SV2C from SV2A and SV2B remains a mystery. Prior studies reveal that the receptor-binding domain of BoNT/A (H_{C}A) recognizes the open edge of the most C-terminal β-strand of the quadrilateral b-helix fold of SV2C luminal domain as well as the core saccharides of a neighboring N-glycan of SV2C, which together form a composite binding site for H_{C}A. However, the receptor-binding domain of BoNT/E (H_{C}E) has an 8-amino acid deletion and many substitutions at the homologous H-A-like SV2-binding site (FIG. 8A), suggesting that BoNT/E exploits a distinct yet unknown mechanism to recognize SV2A and SV2B.

Described herein, the Inventors designed and characterized a fusion protein composed of the luminal domain of human SV2A and SV2C (named SV2Ac) that faithfully maintains a SV2A-like binding capacity to BoNT/E. SV2Ac has better biochemical behavior in comparison to SV2A, which is needed to facilitate subsequence biochemical and structural studies. A fusion protein was also designed consisting of SV2Ac and a single-domain camelid antibody (a.k.a. VHH or nanobody, named G6) that binds to the ganglioside-binding site on H_{C}E and acts as a ganglioside surrogate to enhance H_{C}E-SV2A association. Two crystal structures were then determined of H_{C}E in complexes with SV2Ac-VHH-G6 and SV2Ac-VHH-G6-sialic-acid. These structures revealed that BoNT/E simultaneously recognizes both the specific protein fragments and an N-glycan of SV2A at two separated H_{C}E sites. Complementary biophysical, cellular, and functional studies demonstrated that BoNT/E specifically recognizes SV2A and SV2B, but not SV2C, via the protein-protein interface, while it also grips the tip of the SV2 glycan at a distant site that strengthens association. Both of the protein-protein and protein-glycan binding modes between BoNT/E and SV2A are distinct from that between BoNT/A and SV2C. These findings provide the structural basis to facilitate the therapeutic development and engineering of BoNT/E for novel neurotoxin products, as well as to inform new strategies for developing BoNT inhibitors.

VHH-G6 blocks ganglioside binding of BoNT/E: The fourth luminal domain of human SV2A (residues F487-E581, referred to as SV2A-L4) was expressed and purified, which was previously identified as the BoNT/E-binding fragment, as a secreted and glycosylated protein from human embryonic kidney 293 cells (HEK293). However, HE was found to poorly bind SV2A-L4 and their interaction was barely detectable using a pull-down assay, making it unfeasible for structural studies. It was hypothesized that the low affinity between H_{C}E and SV2A-L4 was due to the lack of co-receptor gangliosides that are known to be essential for the cell entry of BoNT/E. Complex gangliosides are present abundantly on nerve cell surfaces and serve to enrich toxins during the crucial early stage of cell binding. As BoNT/E-receptor recognition on cell surfaces relies on two receptors, described herein are strategies to enhance SV2A binding to HE by conjugating SV2A-L4 with a ganglioside-mimicking component in order to facilitate structural studies. Such an engineered protein should then bind H_{C}E in a manner resembling the dual receptors of BoNT/E. To this end, a VHH (G6) was identified that recognizes the ganglioside-binding site on BoNT/E as a surrogate for gangliosides.

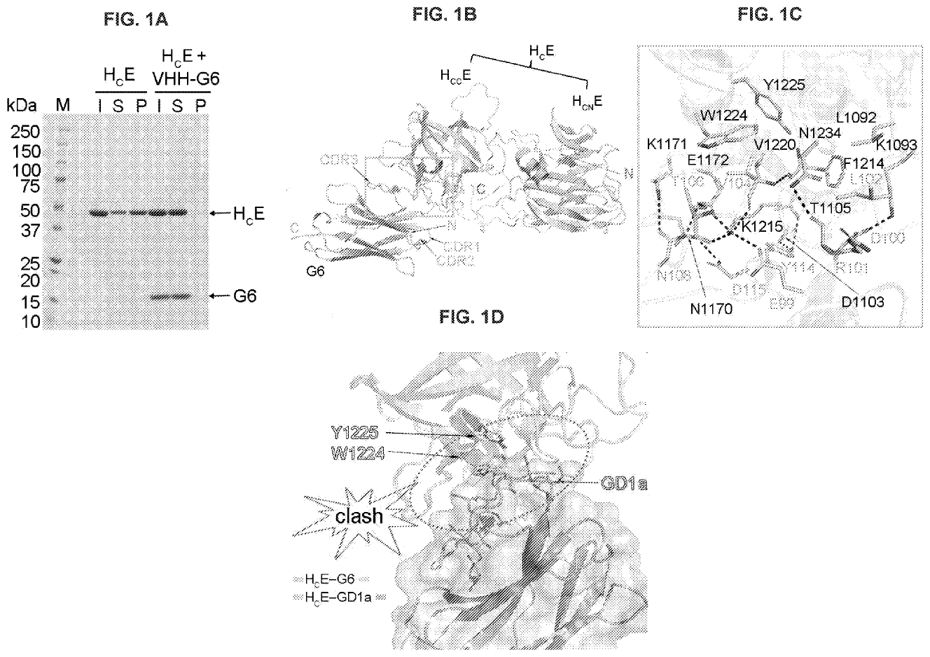

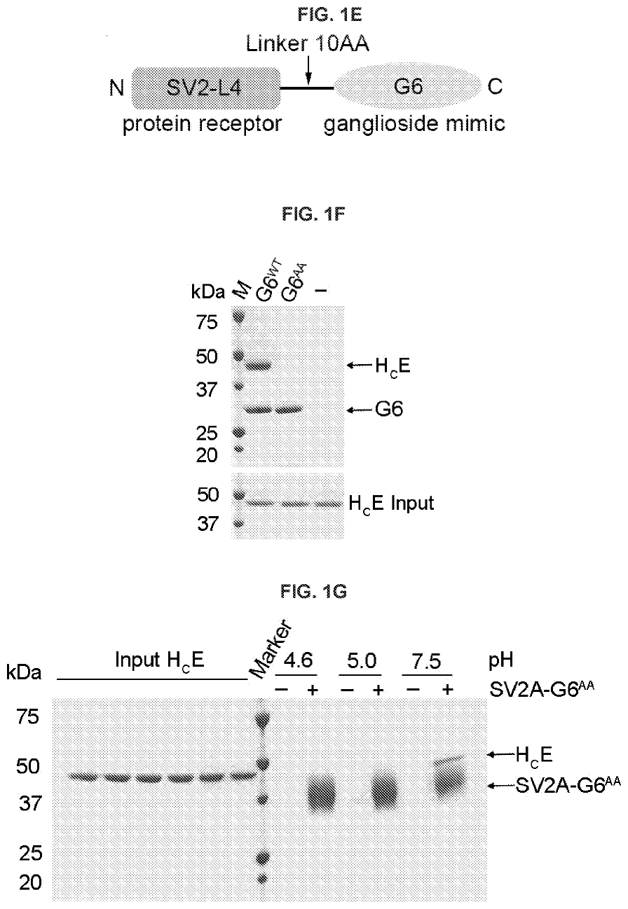

The Inventors have developed numerous BoNT-binding VHHs as reagents and countermeasures, especially for BoNT/A, B, and E which cause the majority of human intoxications. Among many BoNT/E-targeting VHHs, VHH-JLE-G6 (referred to as G6) was focused on, which neutralizes BoNT/E toxicity and displays high affinity binding to H_{C}E. G6 markedly reduced the binding of H_{C}E to liposomes containing complex ganglioside GT1b in a co-sedimentation assay, suggesting that G6 likely competes with GT1b for H_{C}E binding (FIG. 1A). To better understand the neutralizing mechanism of G6, the co-crystal structure was determined of an H_{C}E-G6 complex at 3.23 Å resolution (FIG. 1B and Table 4). This structure reveals that the complementarity-determining region 3 (CDR3) of G6 forms extensive interactions with the C-terminal subdomain of H_{C}E (H_{CC}E) via a network of hydrogen bonds complemented with salt bridges and hydrophobic interactions, while the CDR1 and CDR2 do not directly bind H_{C}E (FIG. 1C). Since the ganglioside-binding modes are highly conserved among different BoNT serotypes, structural modeling was done based on the published structures of H_{C}A and H_{C}B in complex with gangliosides and found that G6 residues V104 and L102 binds to HE at sites that should otherwise accommodate Gal4 and Sia5 of GT1b. As a result, H_{C}E residues W1224 and Y1225, which are part of the highly conserved ganglioside-binding “SxWY” motif, are blocked from binding gangliosides (FIG. 8B). This finding is also consistent with the structure of an H_{C}E-ganglioside complex (PDB: 70VW) (FIG. 1D). Furthermore, mutating H_{C}E-W1224 was sufficient to abolish its ganglioside binding. These results demonstrate that G6 occupies the ganglioside-binding site on H_{C}E and blocks ganglioside binding, leading to BoNT/E neutralization.

TABLE 4
Data collection, phasing and refinement statistics.

| | | | | | H_{C}E-SV2Ac-G6 | |
|---|---|---|---|---|---|---|
| | H_{C}E-G6 | | H_{C}E-SV2Ac-G6 | | (soaking SIA) | |
| | (PDB: 7UIE) | | (PDB: 7UIA) | | (PDB: 7UIB) | |
| Data collection | | | | | | |
| Space group | P 21 21 21 | | C 2 2 21 | | C 2 2 21 | |
| Cell dimensions | | | | | | |
| a, b, c (Å) | 88.74, 174.63, 214.44 | | 140.74, 172.34, 137.16 | | 141.37, 172.18, 137.05 | |
| a, b, g (°) | 90.00, 90.00, 90.00 | | 90.00, 90.00, 90.00 | | 90.00, 90.00, 90.00 | |
| Wavelength (Å) | 0.97918 | | 0.97918 | | 0.97918 | |
| Resolution (Å) | 174.63-3.23 | (3.32-3.23) * | 137.16-2.59 | (2.67-2.59) * | 137.16-2.59 | (2.67-2.59) * |
| R_{merge} (%) | 22.4 | (>100) | 9.8 | (>100) | 9.0 | (55.8) |
| I/σI | 8.6 | (1.0) | 19.9 | (2.6) | 14.6 | (3.2) |
| CC_{1/2} | 0.987 | (0.333) | 0.999 | (0.894) | 0.993 | (0.900) |
| Completeness (%) | 99.6 | (99.7) | 100 | (100) | 99.8 | (99.9) |
| Redundancy | 4.1 | (4.2) | 12.1 | (12.5) | 6.2 | (6.8) |
| Refinement | | | | | | |
| Resolution (Å) | 135.41-3.23 | (3.32-3.23) * | 109.01-2.59 | (2.67-2.59) * | 109.26-2.77 | (2.87-2.77) * |
| No. reflections | 53969 | | 52103 | | 42703 | |
| R_{work}/R_{free} | 23.0/27.3 | | 22.5/25.0 | | 23.5/26.1 | |
| No. atoms | | | | | | |
| Protein | 21293 | | 10169 | | 10189 | |
| Ligand/ion | 0 | | 182 | | 202 | |
| Water | 0 | | 159 | | 178 | |
| B-factors | | | | | | |
| Protein | 91.95 | | 57.21 | | 55.52 | |
| Ligand/ion | — | | 80.87 | | 80.78 | |
| Water | — | | 43.24 | | 44.03 | |
| Ramachandran | | | | | | |
| plot | | | | | | |
| Favored (%) | 95.15 | | 94.6 | | 95.4 | |
| Allowed (%) | 4.85 | | 5.4 | | 4.6 | |
| Outliers (%) | 0 | | 0 | | 0 | |
| R.m.s deviations | | | | | | |

| | | | |
|---|---|---|---|
| Bond lengths (Å) | 0.002 | 0.005 | 0.003 |
| Bond angles (°) | 0.48 | 1.138 | 0.814 |

* Values in parentheses are for highest-resolution shell.
The dataset was derived from a single crystal.

SV2A-G6 fusion protein mimics the dual receptors of BoNT/E: Earlier studies focusing on H_{C}A and H_{C}B demonstrate that the binding sites for their protein receptors and gangliosides are located in two separated but neighboring areas on H_{C} (FIGS. 8C and 8D). The structure of the G6-bound H_{C}E is virtually identical to that in complex with GD1a (root mean square deviation, r.m.s.d. ~0.38 Å over 359 aligned Ca pairs). Therefore, G6 could be used as a ganglioside surrogate to facilitate SV2A binding to H_{C}E when G6 and SV2A are properly connected with a flexible peptide linker because such a fusion protein would allow synergistic binding of G6 and SV2A to H_{C}E in a way resembling the dual receptor binding. Guided by the structure of the H_{C}E-G6 complex, a fusion protein was designed in which G6 (residues Q1-S129) was linked to the C-terminus of SV2A-L4 because the N-terminus of G6 is closer to HE than its C-terminus (FIG. 1E). A 10-amino acid flexible linker that should have sufficient length for SV2A-L4 to sample a large area on H_{C}E surface for binding was employed. To validate this design, a mutated G6 was designed that carries double mutations D100A/D115A on its CDR3 (termed G6^{AA}) to drastically weaken its binding to H_{C}E (FIG. 1F). A properly designed SV2A-G6^{AA} fusion protein that structurally allows the simultaneous binding of both weak binders would display substantially enhanced avidity due to the bivalent binding, while an improperly designed fusion protein in which only one component could bind would display poor affinity. Using a pull-down assay, the glycosylated SV2A-G6^{AA} expressed in HEK293 cells strongly interacted with H_{C}E (FIG. 1G), suggesting that SV2A-G6^{AA} successfully mimics the dual-receptor binding to HE in vitro. Interestingly, SV2A-G6^{AA} robustly bound H_{C}E at neutral pH (e.g. 7.5) but not at acidic pH (e.g. 4.6 and 5.0) (FIG. 1G), which is similar to BoNT/A binding of SV2C but different from the pH-independent binding between BoNT/B and its receptor synaptotagmin.

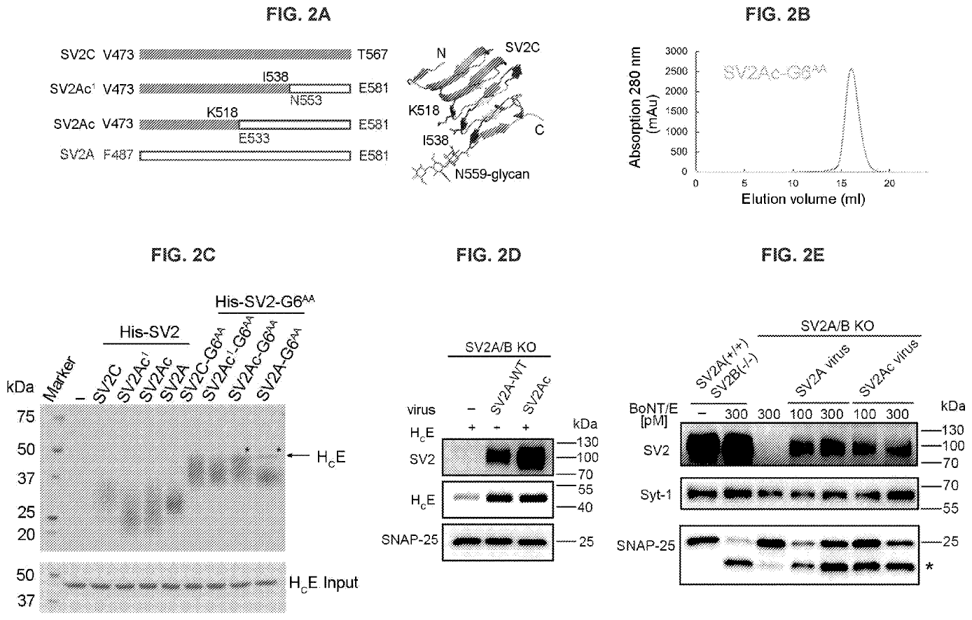

Engineering a SV2A-SV2C chimera capable of recognizing H_{C}E: Systematic screenings were carried out of co-crystallization of H_{C}E in complex with SV2A-G6 in which the wild-type G6 was used to further enhance complex stability. However, despite extensive efforts, high-quality crystals for diffraction studies were unable to be obtained, which was identified largely due to the tendency of SV2A-L4 to aggregate in solution. Interestingly, the recombinant SV2C-L4 is mono-dispersed and has excellent biochemical behavior. As SV2A-L4 and SV2C-L4 are homologous to each other, an SV2A-SV2C chimera was sought to be developed that has improved biochemical behavior over SV2A-L4 while maintaining the SV2A-like binding with H_{C}E. To this end, a series of SV2A-SV2C chimeras were designed in the context of the SV2A-G6^{AA} fusion protein by swapping fragments of SV2A and SV2C, expressed them in HEK293 cells, and then examined their biochemical features and interaction with H_{C}E (FIG. 2A). A chimera composed of the N-terminal segment of SV2C-L4 (V473-K518) and the C-terminal segment of SV2A-L4 (E533-E581) maintained SV2A-like binding to H_{C}E based on the pull-down assay (termed SV2Ac-G6^{AA}), and it was mono-dispersed in solution (FIGS. 2B and 2C). In comparison, another chimera composed of residues V473-1538 of SV2C and N553-E581 of SV2A (SV2Ac1-G6^{AA}), as well as the stand-alone SV2C, SV2Ac, SV2Ac1, SV2A, or SV2C-G6^{AA} did not show detectable binding to H_{C}E in this assay (FIG. 2C). These results suggest that most of the H_{C}E-interacting region is located in the middle to C-terminal portion of SV2A-L4.

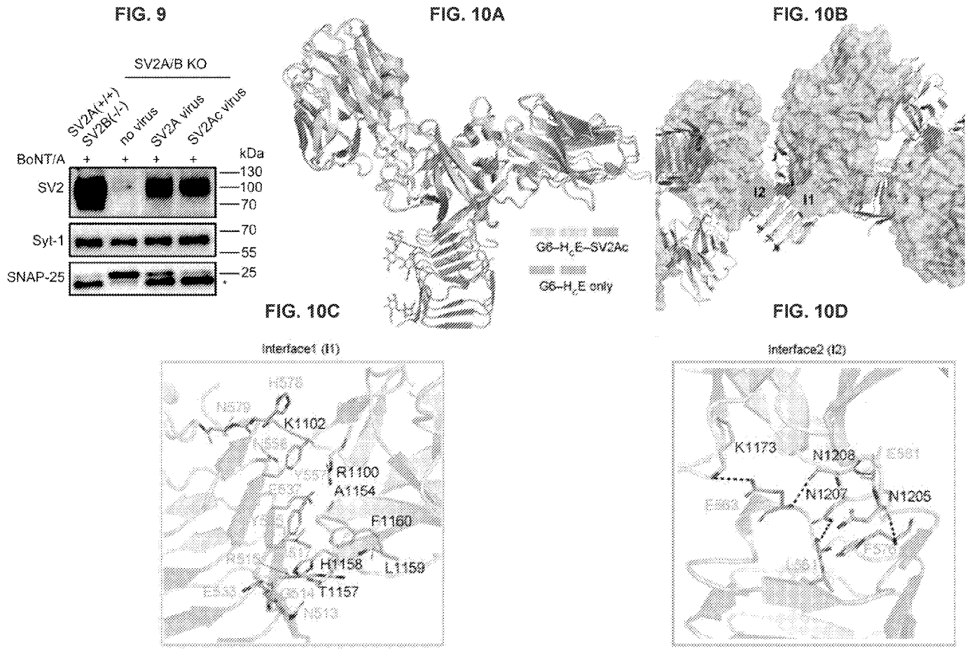

To further validate this finding with full-length SV2 in neurons, SV2A was expressed containing either the wild-type SV2A-L4 or SV2Ac-L4 via lentiviral transduction in cortical neurons cultured from SV2A/B double knockout (KO) mice. These neurons mainly express SV2A and SV2B, but not SV2. The expression of SV2A and SV2Ac mediated similar levels of H_{C}E binding to neurons (FIG. 2D). Furthermore, both SV2A and SV2Ac were able to mediate cell entry of BoNT/E and BoNT/A, resulting in cleavage of their neuronal substrate SNAP-25 (FIG. 2E and FIG. 9). Taken together, these results demonstrate that SV2Ac maintains a SV2A-like binding capacity to BoNT/E on neurons.

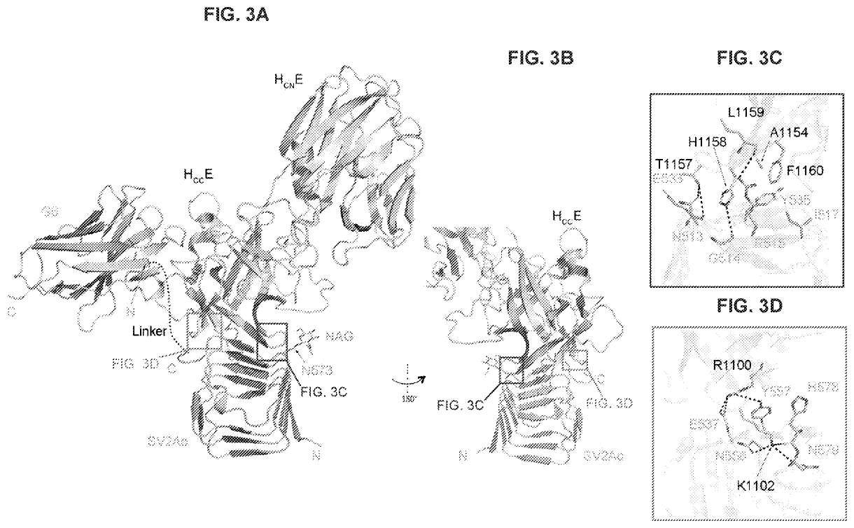

The structure of H_{C}E in complex with SV2A: After prolonged efforts to rationally design and optimize a unique molecule that mimics the dual receptors of BoNT/E, the crystal structure of H_{C}E in complex with SV2Ac-G6 at 2.59 Å resolution was successfully determined (Table 4). There are two pairs of identical H_{C}E-SV2Ac-G6 complexes in one asymmetric unit, with each H_{C}E bound with one molecule of SV2Ac and one G6 (FIGS. 3A and 3B). The peptide linker between SV2Ac and G6 has no visible electron density, indicating a highly flexible conformation. G6, in the context of SV2Ac-G6 fusion protein, binds H_{C}E in the same manner as the stand alone G6, which further demonstrates that the peptide linker did not constrain SV2Ac and G6 association with H_{C}E (FIG. 10A).

The structure of SV2Ac-bound HE is virtually identical to that in the context of BoNT/E holotoxin (PDB: 3FFZ, r.m.s.d. ~0.4 Å over 357 aligned Ca pairs), suggesting the SV2-binding interface is largely pre-organized on BoNT/E. SV2Ac adopts a right-handed, quadrilateral b-helix fold, which is highly similar to SV2C-L4 observed in the H_{C}A-SV2C complex with a r.m.s.d. of ~0.3 Å between comparable Ca atoms (PDB: 5JLV). However, H_{C}E binds to the side of the β-helical bundle of SV2Ac, which is in contrast to HA, which recognizes the open edge of the C-terminal β-strand of SV2C-L4 (FIG. 8C). This binding mode is consistent with the biochemical data showing that replacing the N-terminal region of SV2A with SV2C-like residues did not affect H_{C}E binding (FIG. 2C).

The SV2Ac-binding interface on HE is ~544 Å2, which is completely located on H_{CC}E. It is composed of a central core interface involving extensive hydrogen bonds and hydrophobic interactions that are mediated by residues A1154, T1157, H1158, L1159, and F1160 of H_{C}E and residues N513, G514, R515, 1517, E533, and Y535 of SV2Ac (FIG. 3C), and a separated interface where HE residues R1100 and K1102 establish hydrogen bonds with SV2Ac residues E537, Y557, N558, H578, and N579 (FIG. 3D and FIG. 10C and Table 5). Consistent with the structural findings, mutating H_{C}E residues associated with this interface, such as HER1100G H_{C}E^{R1100G/K1102G}, H_{C}E^{H1158G}, H_{C}E^{T1157A/H1168G}. and H_{C}E^{A1154G/F1160G} abrogated binding to SV2Ac-G6^{AA} in pull-down assays, which will be further discussed in a later section (FIG. 10E). A second interface between H_{C}E and SV2Ac was attributed to a non-physiological crystal packing effect based on observation that mutating key H_{C}E residues at this interface, such as K1173, N1207, and N1208, did not affect its interaction with SV2Ac-G6^{AA} in pull-down assays (FIGS. 10B, 10D, and 10E). On SV2Ac, all H_{C}E-interacting residues are located on one side of the β-helical bundle, and they are all native SV2A residues except for two amino acids located on the SV2C part of the chimera. Specifically, residue G514^{SV2Ac} (equivalent to D514^{SV24} and G500^{SV29}) forms a main-chain-mediated hydrogen bond with H_{C}E-H1158; R515^{SV2Ac} (equivalent to R5015V29), which forms a hydrogen bond with H_{C}E-L1159, has a homologous substitution K515 on SV2A (FIG. 3C), When D514^{SV2A} was replaced with a SV2C-like Gly, D514G^{SV2A} could still maintain WT-like binding to H_{C}E (FIG. 10F), and a structural modeling showed that a Lys at R515^{SV2Ac} would not affect HE binding. Therefore, SV2Ac mimics the WT SV2A when recognizing H_{C}E.

TABLE 5
Protein-protein interactions in the BoNT/E-SV2Ac complex. The amino
acids on BoNT/A that are structurally equivalent to the SV2A-binding
residues on BoNT/E are listed for comparison, while the BoNT/E-
interacting residues on human SV2A and SV2B, and the equivalent
residues on SV2C are also listed. The conserved residues are in
normal text, the homologous substitutions are italicized, while
non-homologous replacements are bold italicized. The underlined
residues are involved in main-chain-mediated interactions.

| | | Type of | | | | |
|---|---|---|---|---|---|---|
| | | inter- | | | | |
| BoNT/A | BoNT/E | action | SV2Ac | SV2A | SV2B | SV2C |
| E1190 | T1157 | HB | N513 | N513 | N456 | N499 |
| Y1191 | H1158 | HB | G514 | D514 | D457 | G500 |
| | | | (mc) | | | |
| R1192 | L1159 | HB | R515 | K515 | K458 | R501 |
| | (mc) | | | | | |
| L1193 | F1160 | vdW | I517 | I517 | T460 | I503 |
| E1190 | T1157 | HB | E533 | E533 | E476 | S519 |
| | | | (mc) | | | |
| L1193/ | A1154/ | vdW | Y535 | Y535 | Y478 | T521 |
| K1187 | F1160 | | | | | |
| V1125 | R1100 | SB | E537 | E537 | E480 | E523 |
| | | HB | Y557 | Y557 | Y500 | D543 |
| N1127 | K1102 | HB | N558 | N558 | N501 | N544 |
| | | HB | H578 | H578 | E521 | H564 |
| | | | (mc) | | | |
| | | HB | N579 | N579 | Q522 | N565 |
| | | | (mc) | | | |

“SB”, “vdW”, and “HB” stand for salt bridge, van der Waals interaction, and hydrogen bond, respectively. “mc” indicates the main-chain-mediated contacts, and all the other contacts are mediated by side-chain atoms.

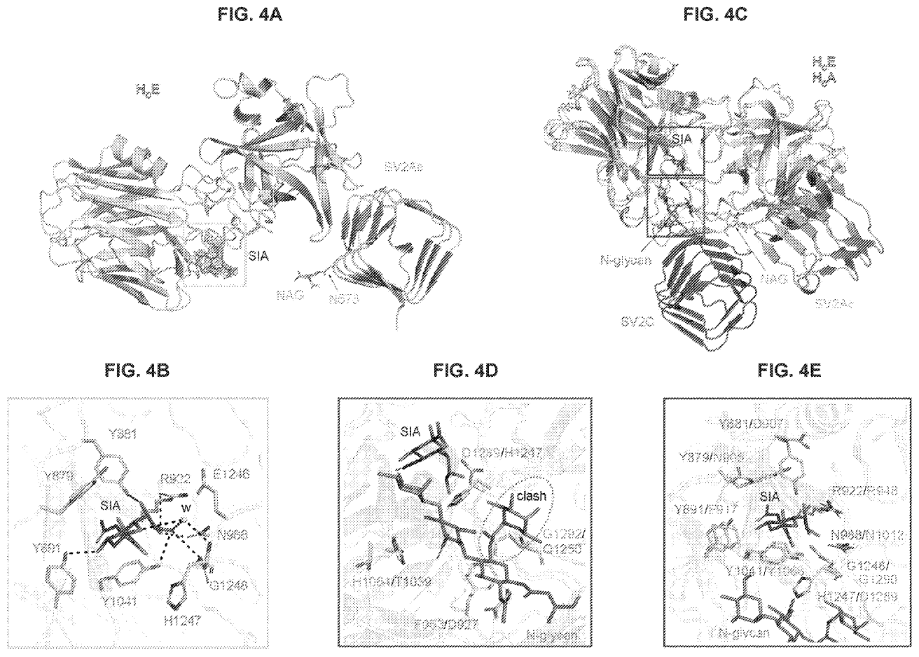

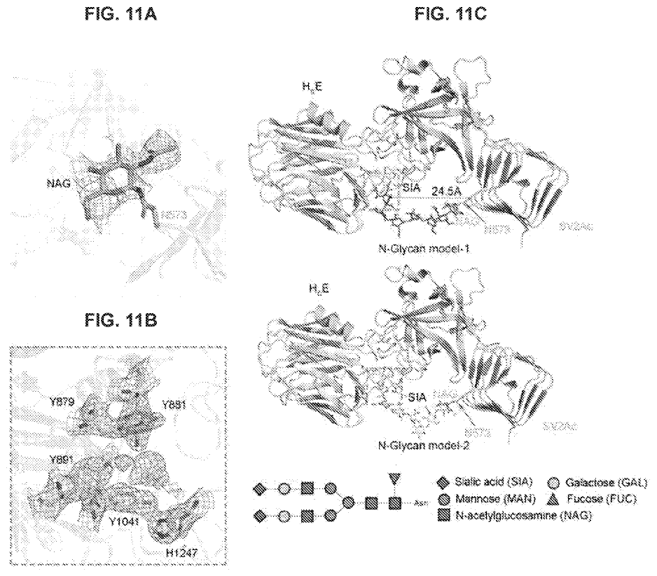

BoNT/E grips the sialic acid of SV2A glycan: An N-glycan that is highly conserved on SV2A (N573), SV2B (N516), and SV2C (N559) across vertebrates is crucial for cell entry of BoNT/A and BoNT/E. In the structure of the H_{C}E-SV2Ac-G6 complex, only the electron density for the core N-acetylglucosamine (NAG) of this crucial glycan linked to SV2A-N573 was observed (FIG. 11A). Notably, this NAG is pointing away from the protein-protein interface between H_{C}E and SV2A (FIG. 4A), and given this binding mode, the rest of SV2A-N573 glycan core is unlikely able to interact with the neighboring H_{C}E residues. This is in sharp contrast to the glycan-binding mode of H_{C}A in which the quadruple-saccharide core of the N-glycan attached to SV2C-N559 is located next to the protein-protein interface where it can be conveniently gripped by HA via extensive interactions to enhance protein-based H-A-SV2C binding (FIG. &C). These findings suggest that the SV2A-N573 glycan may adopt an unconventional H_{C}E-binding mode that is technically challenging to be defined by co-crystallization. This study was further complicated by the appearance of SV2Ac-G6 as smeared bands on SDS-PAGE gels representing heterogeneous glycoforms (FIG. 2C). This was not unexpected as recombinant glycoproteins expressed in HEK293 cells typically contain heterogeneous glycosylation under over-expression conditions.

During late stage structure refinement, electron densities for an unknown molecule located in a pocket formed by four tyrosine residues (e.g. Y879, Y881, Y891, and Y1041) at the N-terminal sub-domain of H_{C}E (H_{CN}E), which is about ~25 Å away from N573 of SV2A was noticed (FIG. 11B). Coincidentally, mutating residues Y879 or Y1041 on BoNT/E to an alanine led to a dramatic 99% reduction of its neurotoxicity. That observation could not be readily explained by any known mechanism, because all the known protein receptor- and ganglioside-binding sites are located on H_{CC}E. Based on structural modeling studies, a complex type of N-glycan attached to N573 of SV2A given the structure of the H_{C}E-SV2Ac complex could reach this distant pocket on HONE (FIG. 11C). Moreover, besides the four Tyr residues, it was noticed that there are several other hydrophobic H_{C}E residues (e.g., Y926 and H1247) in this area that are also well suited for carbohydrate binding. Taken together, this unknown density could represent the distant portion of the SV2A-N573 glycan chain, which had a low occupancy at this remote site partly due to glycan heterogeneity.

The obscure electron density observed for this putative glycan indicated weak interactions that would be impractical to be characterized by direct binding studies. Therefore systematic crystal soaking screens were carried out using component sugars of a typical complex type N-glycan, including monosaccharides sialic acid (Neu5Ac), N-acetylglucosamines (GlcNAc), galactose (Gal), and a disaccharide N-acetyl-D-lactosamine (Galβ1-4GlcNAc, LacNAc) that is the smallest repeating unit in most N-glycans. Based on a 2.77 Å resolution structure of a sialic acid soaked H_{C}E-SV2Ac-G6 crystal, a sialic acid could clearly be seen occupying this mysterious pocket on HONE (FIG. 4A and Table 4). The electron densities at this site for all other sugars that were carried out in parallel crystal soaking studies were similar to the un-soaked crystals and could not be modeled. Structurally, this sialic acid is sandwiched between Y879 and Y1041 on H_{CN}E, surrounded by Y881, Y891, and H1247, and with associations further strengthened by several hydrogen bonds with R922 and N988 on H_{CN}E and G1248 of H_{CC}E (FIG. 4B). These BoNT/E residues are discontinued in the primary sequence, but converge in 3D to form a pocket that accommodates a sialic acid that is frequently found to cap the termini of oligosaccharide chains of N-glycans. These structural findings suggest that BoNT/E appears to grip the terminal sialic acid of the SV2A-N573 glycan at a site that is distant (~25 Å away from N573 of SV2A) from the main protein-protein interface.

BoNT/E and BoNT/A exploit distinct glycan-binding modes: A structural comparison between the glycan-bound H_{C}E and HA revealed that the sialic acid-binding site on H_{C}E is located close to the glycan-binding site on H_{C}A (FIG. 4C). The glycan-binding residues on H_{C}A are not preserved on H_{C}E. For example, H-A-G1292 is substituted by H_{C}E-Q1250 whose large side chain would clash with the SV2C glycan (FIG. 4D). This may preclude H_{C}E from using a H_{C}A-like glycan-binding mode. Therefore, this region of HE should be modified in order to allow the engineered HE adopts a H-A-like receptor-binding mode (for example, amino acid “QEK” of the wild-type HE was replaced with “GERPL” in H_{C}E-V3.5, (SEQ ID NO: 3). On the other hand, the sialic acid-binding site on H_{C}E is partially conserved on HA (FIG. 4E), raising the possibility that BoNT/A might use this H_{C}E-like site to recognize the terminal sialic acid of the SV2C glycan. However, the structural modeling reveals that the glycan anchoring residue N559 of the H_{C}A-bound SV2C is located very close to this hypothetic HE-like sialic acid-binding site (FIG. 4C), and as a result, the terminal sialic acid of the H_{C}A-bound SV2C N559 glycan would be located beyond this hypothetical sialic acid-binding pocket on H_{C}A. In an earlier mutagenesis studies on BoNT/A, mutating H_{C}A residues N905, F917, and D1289, which are equivalent to the sialic acid-binding residues Y879, Y891, and H1247 on H_{C}E, displayed only moderately decreased neurotoxicity. These results suggest that H_{C}A does not use a H_{C}E-like glycan-binding mode, while the core saccharide of the SV2C-N559 glycan together with the protein moiety of SV2C play a dominant role in mediating BoNT/A binding.

Simultaneous binding to the protein- and glycan-moiety of SV2A is crucial for BoNT/E function: Structure-based mutagenesis studies were then carried out to validate the structural findings and to further characterize the functional role of BoNT/E-SV2A interplays. Guided by the crystal structures, H_{C}E variants were designed that carry two different types of mutations: (1) mutations that weaken H_{C}E binding to SV2A protein moiety, including H_{C}E^{R1100G} H_{C}E^{K1102G}, H_{C}E^{R100G/K102G}, H_{C}E^{T1157A}, H_{C}E^{T1157A/H1158G}, H_{C}E^{A1154G/F1160G}, and(2) mutations that disrupt H_{C}E association with sialic acid, including H_{C}E^{Y879G/Y881G}, H_{C}E^{Y891G/Y1041G}, and H_{C}E^{E1246A/H1247A}. First it was confirmed that all these mutations did not alter H_{C}E folding and stability based on thermal denaturation experiments (FIG. 12). Then two sets of studies were carried out to examine how these H_{C}E mutants recognized SV2A-G6^{AA} in vitro using pull-down assays (FIG. 10E) and endogenous SV2A and SV2B in cultured rat cortical neurons (FIG. 5A). H_{C}E^{R1100G}, H_{C}E^{R1100G/K1102G}, H_{C}E^{H1158G}, H_{C}E^{T1157A/H1158G}, and H_{C}E^{A1154G/F1160G} that have the disrupted protein-based SV2A-binding interface showed largely abolished binding to SV2A-G6^{AA} in vitro and endogenous SV2 on rat cortical neurons. Furthermore, mutating the sialic acid-binding residues in H_{C}E^{Y879G/Y881G}, H_{C}E^{Y891G/Y1041G}, and H_{C}E^{E1246A/H1247A} also largely reduced their binding to SV2 on neurons (FIG. 5A). A detectable change of binding between SV2A-G6MA and H_{C}E^{Y891G/Y1041G} or H_{C}E^{Y891G/Y1041G} in vitro using pull-down assays was not observed (FIG. 10E), which could be due to the heterogeneous glycosylation of the recombinant SV2A that is different from the glycosylation pattern of SV2A on neurons. Therefore, the in vitro pull-down assay was mostly detecting the protein-mediated interactions. Taken together, these data suggest that both the protein- and the glycan-mediated associations are necessary for H_{C}E-SV2A recognition on neuronal surfaces.

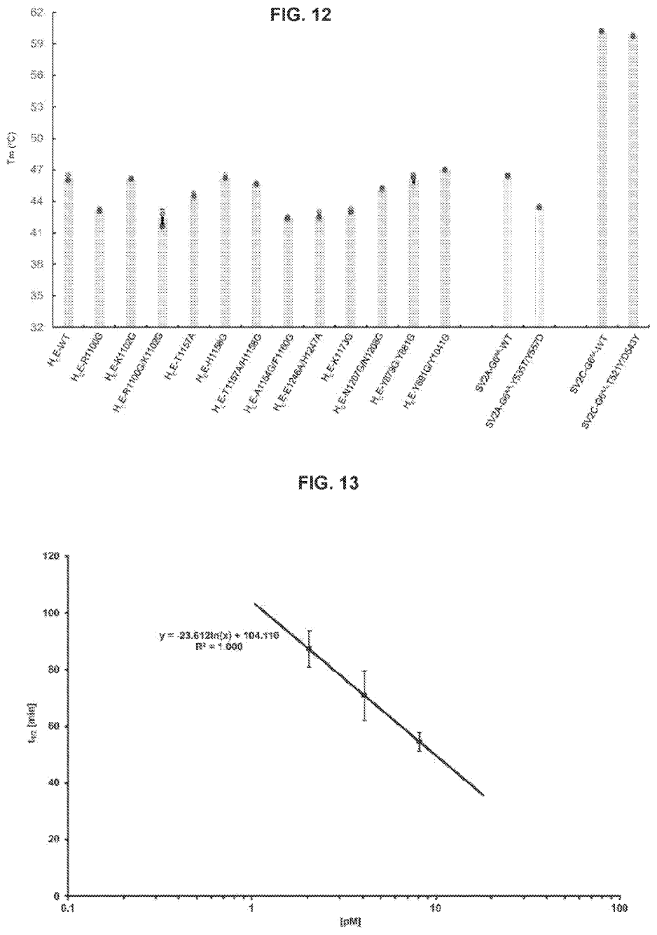

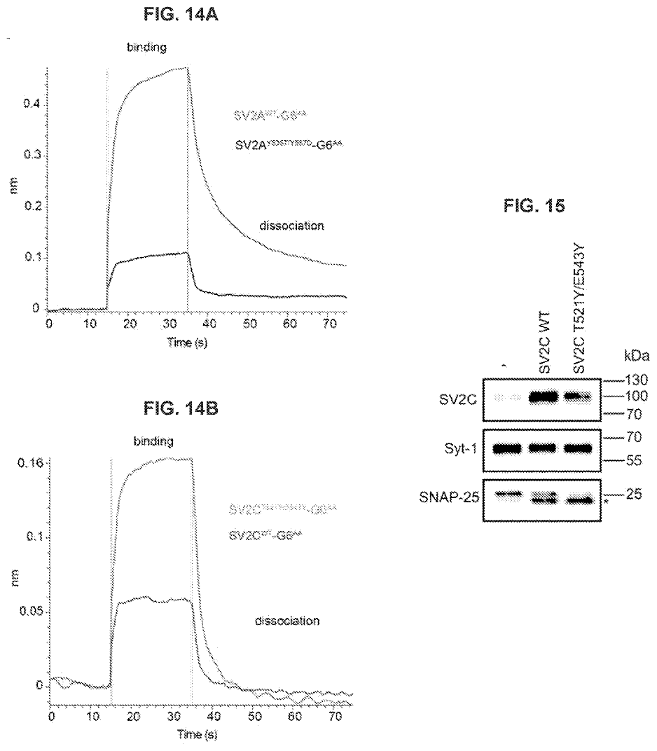

To further establish the physiological relevance of the protein- and glycan-mediated H_{C}E-SV2A interactions, four SV2A-binding deficient mutants of the full-length BoNT/E were produced based on the results of the mutagenesis studies on H_{C}E described above, and examined their neurotoxicity at motor nerve terminals using an ex vivo mouse phrenic nerve hemi-diaphragm (MPN) assay (FIG. 5B and FIG. 13). BoNT/E^{R1100G/K1102G} and BoNT/E^{H158G/F1160G}, which have mutations at two separated sites of the protein-protein interface with SV2A, showed ~90% decreased toxicity, while BoNT/E^{R1100G/H1158G/F1160G} that carries mutations at both sites showed a further decrease to ~99% (FIG. 5B). To examine the functional role of glycan-mediated interactions, BoNT/E^{Y879G/1041G} were designed based on H_{C}E^{Y879G/Y881G} and H_{C}E^{Y891G/Y1041G}, in which both Y879 and Y1041 of BoNT/E that sandwich the sialic acid were mutated. Remarkably, BoNT/E^{Y879G/Y1041G} only retained ~0.1% neurotoxicity despite its intact binding site for the protein moiety of SV2A, strongly supporting the direct involvement of the N-glycan of SV2A and SV2B in BoNT/E binding and function. The destructive effects of mutations at the glycan-binding site of BoNT/E were stronger as revealed by the MPN assay in comparison to the results of neuron binding assay based on H_{C}E (FIG. 5A), which is likely due to the different functional read out sensitivity of the two assays and the different amount of BoNT/E (WT at 2-8 μM and mutants at 20 μM-6 nM) and H_{C}E (200 nM) used. Together, these data demonstrate that both the protein and glycan moieties of SV2A are essential for the neurotoxicity of BoNT/E at motor nerve terminals.

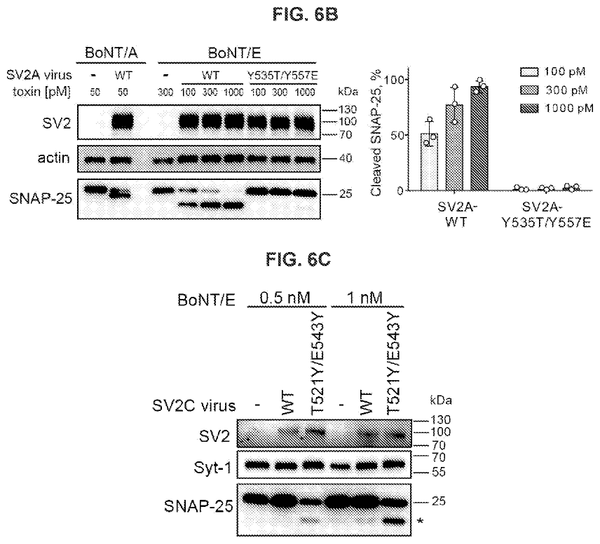

BoNT/E selectively recognizes SV2A and SV2B, but not SV2C: BoNT/E can utilize SV2A and SV2B, but not the closely related SV2C, as receptors in hippocampal and cortical neurons. Since both SV2A/SV2B and SV2C have a conserved N-glycan at the same location (N573^{SV2A}, N516^{SV2B}, and N559^{SV2C}), HE may distinguish SV2A/SV2B from SV2C mainly based on amino acid differences at the protein-protein interface. Structure-based sequence analyses revealed that the BoNT/E-binding residues are mostly identical between SV2A and SV2B except for three subtle amino acid substitutions, which are H578^{SV2A} and N579^{SV2A} that form main-chain-mediated hydrogen bonds with H_{C}E-K1102, and I517^{SV2A} that packs against the hydrophobic H_{C}E-F1160. These three SV2A residues are replaced by E521^{SV2B}, Q522^{SV2B}, T460^{SV2B}, respectively, which should not have a major effect on H_{C}E binding (FIG. 6A and Table 5). In contrast, Y535^{SV2A}/Y478^{SV2B} located at the core of BoNT/E-SV2 protein-protein interface is replaced with T521^{SV2C}, which will weaken the hydrophobic packing with H_{C}E-F1160, and the interaction between Y557^{SV2A}/Y500^{SV2B} and H_{C}E-R1100 will be disrupted by the corresponding residue D543^{SV2C} in human/mouse (homologous E543^{SV2C} on rat) (FIG. 6A and Table 5).

To test these predictions, residues Y535 and Y557 were swapped on SV2A-L4 with the corresponding residues on SV2C-L4 to generate a “SV2C-like” SV2A^{Y535T/Y557D} and vice versa to generate a “SV2A-like” SV2C^{T521Y/O643Y}. G6^{AA} was first linked with them and examined how they recognized HSE in vitro using a bio-layer interferometry (BLI) assay. SV2A^{Y535T/Y567D}-G6^{AA} showed a markedly decreased binding to H_{C}E vs. SV2A-G6^{AA}, while SV2C^{T521Y/O543Y}-G6^{AA} showed a clearly improved binding to H_{C}E vs. SV2C-G6^{AA} (FIGS. 14A and 14B). To better understand the physiological relevance of this structural finding, these two mutants were expressed as full-length SV2 (SV2A^{Y335T/Y567E} and SV2C^{T521Y/E543Y}, the rat SV2 genes that has E543 on SV2C were used in this experiment) in cortical neurons cultured from SV2A/SV2B KO mice via lentivirus transduction. Using the wild type BoNT/E, the SV2A^{Y535T/Y557E} mutant lost its function to mediate toxin entry at three different toxin doses tested (FIG. 6B). Expression of SV2C^{T521Y/E543Y} mediated a low level of entry of BoNT/E, resulting in a minor cleavage of SNAP-25 at two toxin doses tested, whereas over-expression of WT SV2C did not mediate entry of BoNT/E (FIG. 6C). Both SV2C^{T521Y/E543Y} and WT SV2C mediated entry of BoNT/A (FIG. 13). These results suggest that SV2C^{T521Y/E543Y} gained the capability to mediate BoNT/E entry, albeit at a low efficacy. Additional mutations might be needed to further enhance binding of BoNT/E to SV2C^{T521Y/E543Y}. These findings suggest that BoNT/E is able to detect the subtle differences in the primary sequences of SV2A/2B and SV2C, even though the overall structures of SV2A and SV2C are similar. In contrast, BoNT/A recognizes all three SV2 isoforms because there are mostly backbone-to-backbone interactions between BoNT/A and SV2 at the protein-protein interface that tolerate residue changes across SV2 isoforms.

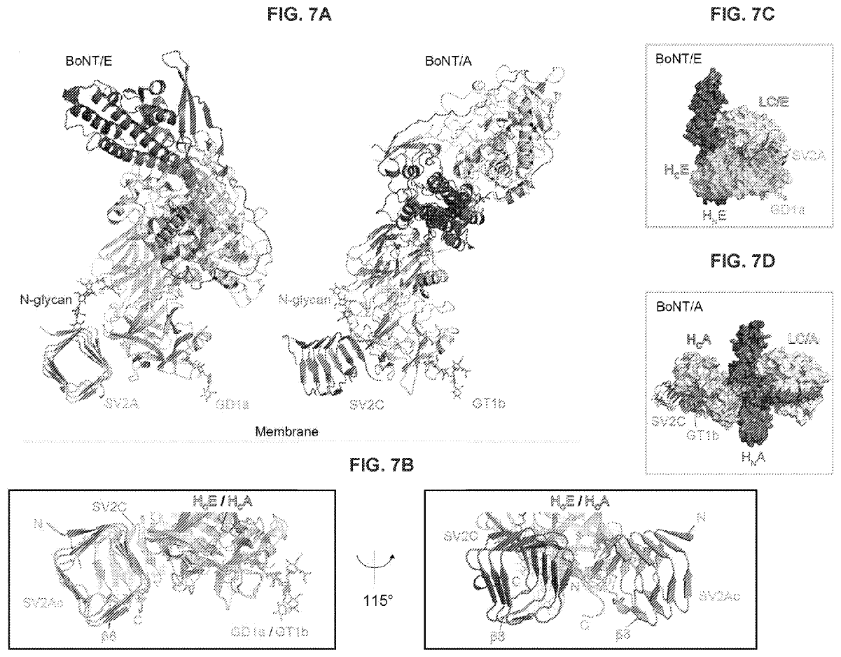

BoNT/E together with BoNT/A and BoNT/B are the major causes of human botulism. Paradoxically BoNT/A and BoNT/B are also approved drugs for a myriad of therapeutic and aesthetic uses. Due to its unique pharmacological and clinical profiles, BoNT/E has attracted growing therapeutic interests and is currently in clinical trials for new indications that may benefit from BoNT/E's faster onset of action and shorter duration. Here, the crystal structure of H_{C}E was determined in complex with a fusion protein of human SV2A and SV2C (SV2Ac), which reveals two distant receptor-binding sites that are well separated on the two subdomains of H_{C}E: the major interface is between H_{CC}E and the side of the quadrilateral β-helix of SV2A-L4, while the SVZA-N573 glycan extends toward HONE with one of its terminal sialic acids buried in a hydrophobic pocket on H_{CN}E (FIG. 7A). This is distinct from BoNT/A, which uses a composite interface located between H_{CN}A and H_{CC}A to recognize both the protein component of SV2C and the core saccharides of a neighboring N-glycan (FIG. 7A and FIG. 8C). Further structure-based mutagenesis and functional studies demonstrate that both the protein- and N-glycan-based engagements are crucial for SV2A-mediated binding and entry of BoNT/E into neurons (FIGS. 5A and 5B and FIG. 10E).

In contrast to viruses and other toxins that typically use multivalent-binding modes to compensate for the weak association at each individual protein-glycan interface, BoNT/E makes use of an independent protein-protein interface to not only enhance glycan-mediated binding to SV2, but also simultaneously provide the crucial specificity information to determine its tissue tropism (FIG. 7A-7D). There are several amino acid substitutions at the protein-mediated SV2A-binding interface on H_{C}E among twelve known BoNT/E subtypes (BoNT/E1-E12). For example, a key SV2A-binding residue R1100 is replaced with S1100 on subtype BoNT/E10 and E11, which could weaken receptor binding and may be partly responsible for the reported lower toxicity of culture supernatants containing BoNT/E10 and E11 besides other factors such as growth rate and toxin secretion (FIG. 16). Therefore, mutating S1100 to Arg or a similar amino acid on BoNT/E10 and E11 is expected to improve their potency. However, the SV2 glycan-binding sites are highly conserved in all twelve BoNT/E subtypes (FIG. 16), which should allow certain tolerance for amino acid changes at the SV2 protein-binding site of BoNT/E during evolution. Furthermore, this glycan-binding site on BoNT/E is partially preserved on BoNT/A, which is very close to the known glycan-binding site on H_{C}A that accommodates the core saccharides of the SV2C N559 glycan, suggesting possible additional interactions between BoNT/A and this SV2C glycan.

It is well accepted that the docking orientations of BoNTs, with each toxin composed of a light chain (LC), the translocation domain (H_{N}), and the receptor-binding H_{C}, on the neuronal surface are largely constrained by simultaneous binding of the H_{C} to the membrane-anchored gangliosides and protein receptors. Even though SV2 binds to distinct sites on H_{C}E and H_{C}A and uses different glycan-binding modes, the putative docking orientations of H_{C}E and H_{C}A on cell surface are similar and the relative orientations of the quadrilateral b-helix of SV2A-LC and SV2C-L4 are also similar (FIGS. 7A and 7B). However, in the context of the holo toxins, the LC-H_{N} moiety of BoNT/E and BoNT/A are oriented differently relative to the H, and the membrane. This is because BoNT/A displays a linear “open-wing”-like arrangement where the H_{C} and LC are located on opposite sides of the long helical H_{N}, while the H, and the LC-H_{N} of BoNT/E fold toward each other resulting in a “closed-wing” conformation (FIGS. 7C and 7D). This finding provides the structural basis to inform future studies on how BoNTs may reorganize their three domains after receptor-mediated binding on neuron surface and proceed to transmembrane delivery of the LC to the cytosol, as prior studies suggested that the translocation process is more rapid in BoNT/E than BoNT/A.

SV2A is expressed in a subset of motor neurons, whereas both SV2B and SV2C are detected in the majority of motor neurons. BoNT/E cannot utilize SV2C as a receptor in cultured hippocampal and cortical neurons. Studies described herein provide a structural basis to understand the selectivity of BoNT/E towards SV2A/2B over SV2C, particularly involving their protein sequences, which suggest that SV2C in motor neurons cannot function as a receptor for BoNT/E. The structures suggest a potentially important role of the conserved SV2 N-glycan in mediating BoNT/E interactions, which may also contribute to differences between SV2A/2B and SV2C as BoNT/E receptors.

As the three SV2 isoforms have different tissue distributions in human, the variations in binding affinity toward SV2 isoforms between BoNT/E and BoNT/A may contribute to their distinct pharmacological and therapeutic features. Based on the knowledge revealed in this study showing how HE selectively recognizes SV2A/SV2B, but not SV2C, and the known mechanisms by which H_{C}A recognizes all three SV2 isoforms, it is possible to engineer new H_{C}E variants that have H A-like binding to SV2A/2B/2C with a higher binding affinity. At the same time, one could engineer new H_{C}A variants with H_{C}E-like receptor-binding features. BoNT/E and BoNT/A holotoxin carrying these engineered H_{C}E or H_{C}A variants can be used for new clinical developments. Notably, recent structure-based engineering of BoNT/B successfully enhanced its binding to human receptor synaptotagmin-II and led to improved clinical efficacy. At the same time, the highly conserved glycan-binding pocket among all BoNT/E subtypes is of particular Interest for future development of epitope-focused antibodies for countermeasure of botulism or reversal of muscle paralysis in clinic.

Ethics statement: All animal studies were performed in the Min Dong lab and approved by the Boston Children's Hospital Institutional Animal Care and Use Committee (Protocol Number: 18-10-3794R). All procedures were approved by the Institute of Biosafety Committees at Boston Children's Hospital (Protocol Number: IBC-P00000501). The MPN assay (project license 2018/209) was performed in the Andreas Rummel lab (Hannover Medical School) according to § 4 Abs. 3 (killing of animals for scientific purposes, German animal protection law (TSchG)). Number of animals sacrificed by trained personnel before dissection of organs were reported yearly to the animal welfare officer of the Central Animal Laboratory and to the local authority, Veterinäramt Hannover.

Cloning, expression, and purification of recombinant proteins: The genes encoding H_{C}E (residues R846-K1252) and VHH G6 (residues Q1-S129) were cloned into a modified pET28a vector with a 6×His/SUMO (Saccharomyces cerevisiae Smt3p) tag introduced to the N-terminus. The core regions of human SV2A-L4 (residues F487-E581) and human SV2C-L4 (residues V473-T567) were cloned into a modified pcDNA vector for mammalian cell expression, and a human IL2 signal sequence (MYRMQLLSCIALSLALVTNS), a 9×His tag, a factor Xa-cleavage site and a human rhinovirus 3C protease cleavage site were added to the N-terminus. The chimeric SV2A-SV2C-L4 constructs were generated by two-step PCR and verified by DNA sequencing. Specifically, SV2Ac1 was made by replacing SV2A amino acids F487-1552 with the corresponding SV2C amino acids V473-1538 and SV2Ac was made by replacing SV2A residues F487-E532 with the corresponding SV2C residues V473-K518. The chimeras were cloned into the modified pcDNA vector for expression. For the SV2Ac-G6 fusion protein, G6 or the G6A (D100A/D115A) mutant were covalently linked to the C terminus of SV2Ac through a 10-amino acid peptide linker (GTSPSASGGS) and cloned into the modified pcDNA vector for expression. The other fusion constructs, including SV2A-G6^{AA}, SV2Ac1-G6^{AA} and SV2C-G6^{AA}, were generated in a similar manner. All site-specific mutations were generated by two-step PCR and verified by DNA sequencing.

H_{C}E and VHH G6 (WT and mutations) were expressed in E. coli strain BL21-Star (DE3) (Invitrogen). Bacteria were cultured at 37° C. in LB medium containing kanamycin. The temperature was reduced to 18° C. when OD6 reached ~0.8. Expression was induced with 1 mM IPTG (isopropyl-b-D-thiogalactopyranoside) and continued at 18° C. overnight. The cells were harvested by centrifugation and stored at −80° C. until use.

The 6×His/SUMO-tagged H_{C}E and G6 (WT and mutations) were purified using Ni²⁺-NTA (nitrilotriacetic acid, Qiagen) affinity resins in a buffer containing 50 mM Tris, pH 7.5, 400 mM NaCl, and 40 mM imidazole. The proteins were eluted with a high-imidazole buffer (50 mM Tris, pH 7.5, 400 mM NaCl, and 300 mM imidazole) then exchanged into a buffer containing 50 mM Tris, pH 7.5, and 400 mM NaCl. The 6×His/SUMO tags of H_{C}E and G6 were cleaved by SUMO protease. HE was further purified by MonoS ion-exchange chromatography (GE Healthcare) in a buffer containing 20 mM MES, pH 6.0, 150 mM NaCl, and 1 mM TCEP, and eluted with a NaCl gradient. The peak fractions of H_{C}E were pooled and subjected to Ni²⁺-NTA re-binding, and the flow through was concentrated, frozen in liquid nitrogen, and kept at −80° C. G6 was also further purified by Ni²⁺-NTA re-binding. To obtain the H_{C}E-G6 complex for crystallization, the purified H_{C}E was mixed with G6 for 1-hour incubation, then purified by Superdex-200 SEC (GE Healthcare) in a buffer containing 20 mM HEPES, pH 7.5, and 150 mM NaCl, and the peak fractions were concentrated to ~10 mg/ml for crystallization.

SV2A, SV2Ac¹, SV2Ac, SV2C, SV2A-G6A, SV2Ac¹-G6^{AA}, SV2Ac-G6A, SV2Ac-G6, SV2C-G6^{AA}, and their mutations were expressed and secreted from FreeStyle HEK 293 cells (ThermoFisher) and purified directly from cell culture media using Ni²⁺-NTA resins. To prepare the H_{C}E-SV2Ac-G6 complex for crystallization, the purified H_{C}E and 9×His-tagged SV2Ac-G6 were mixed at a molar ratio of ~2:1 for 2 hours at 12° C. The complex was isolated using Ni²⁺-NTA resins and further purified by Superdex-200 SEC (GE Healthcare) in a buffer containing 10 mM HEPES, pH 7.5, 150 mM NaCl, and 1 mM TCEP. The N-terminal 9×His tag of SV2Ac-G6 in the complex was removed by 3C protease, and the complex was further purified by Ni²⁺-NTA re-binding and concentrated to ~8 mg/ml for crystallization.

The wild-type and mutated recombinant full-length activated BoNT/E1 including BoNT/E-V3.5 were produced under biosafety level 2 containment (project number GAA A/Z 40654/3/123/3) recombinantly in E. coli BL21 DE3 strain in Dr. Rummel's lab in Germany as described previously. All mutations were generated by two-step PCR and verified by DNA sequencing. BoNT/E and mutants carrying C-terminal His6-tag were purified on Co²⁺-Talon matrix (Takara Bio Europe S.A.S., France) and eluted with 50 mM Tris-HCl (pH 8.0), 150 mM NaCl, and 250 mM imidazole. For proteolytic activation and removal of affinity tag, BoNT/E variants were incubated for 16 hours at room temperature with 0.01 U bovine thrombin (Sigma-Aldrich Chemie GmbH, Germany) per μg of BoNT. Subsequent gel filtration (Superdex-200 SEC; GE Healthcare, Germany) was performed in phosphate buffered saline (pH 7.4). For storage, BoNT/E and mutants were shock-frozen in liquid nitrogen and stored at −80° C.

Crystallization: Initial crystallization screens of the H_{C}E-G6 and the H_{C}E-SV2Ac-G6 complex were carried out at 18° C. using a Gryphon crystallization robot (Art Robbins Instruments) with high-throughput crystallization screening kits (Hampton Research and Qiagen). The original crystals of the H_{C}E-G6 complex were obtained in a reservoir containing 0.2 M NaCl and 20% PEG 3350. And the H_{C}E-SV2Ac-G6 complex was originally crystallized in a reservoir containing 0.2 M potassium sulfate and 20% PEG 3350. Extensive manual optimization was then performed using the hanging-drop vapor-diffusion method via mixing the protein with reservoir solution at 1:1 ratio. For the H_{C}E-G6 complex, the best crystals were obtained in a reservoir containing 0.1 M HEPES, pH 7.0, 0.2 M NaCl, and 18% PEG 3350, and the crystals were cryoprotected in the mother liquor supplemented with 20% (v/v) ethylene glycol. The best crystals for the H_{C}E-SV2Ac-G6 complex were obtained in a reservoir containing 0.1 M HEPES, pH 7.5, 0.2 M potassium sulfate, 20% PEG 3350, and 5% PEG 400. Streak-seeding was necessary to obtain single crystals. For the sugar soaking studies, the crystals of the H_{C}E-SV2Ac-G6 complex were soaked in the mother liquor supplemented with 100 mM sialic acid (Neu5Ac), N-acetylglucosamines (GlcNAc), galactose (Gal), or N-acetyl-D-lactosamine (Galβ1-4GlcNAc, LacNAc) at 18° C. overnight. The crystals were then cryoprotected in buffers containing 0.1 M HEPES, PH 7.5, 23% PEG3350, 12% glycerol, 0.16 M potassium sulfate, and the corresponding sugars, and flash-frozen in liquid nitrogen for diffraction studies.

Data collection and structure determination: The X-ray diffraction data were collected at 100 K at the NE-CAT beamline 24-ID, Advanced Photon Source (APS). The data were processed with XDS as implemented in RAPD. The complex structures were solved by molecular replacement software PHENIX.Phaser using the structures of H_{C}E (PDB: 3FFZ), VHH (PDB: 6GLW), and SV2C-L4 (PDB: 5JLV) as the search models. The crystals of the H_{C}E-G6 complex belong to space group P2₁ 2₁ 2₁, and there are five pairs of the H_{C}E-G6 complexes in one asymmetric unit. The crystals of the H_{C}E-SV2Ac-G6 complex belong to space group C2 2 2₁ with two pairs of the H_{C}E-SV2Ac-G6 complexes in the asymmetric unit. The initial atomic models were refined with Phenix.Refinement. Further structural modeling and refinement were carried out iteratively using COOT and Phenix.Refinement or Refmac5 refinement. The structure of the sialic acid-bound H_{C}E-SV2Ac-G6 complex was solved using the H_{C}E-SV2Ac-G6 complex as a model, and sialic acid was modeled based on the F_{O}-F_{C} electron density maps. All the refinement progresses were monitored with the free R value using a 5% randomly selected test set and the structures were validated by MolProbity. Data collection and structural refinement statistics are listed in Table 4. All structure figures were prepared using Pymol (DeLano Scientific).

Liposome co-sedimentation assay: Large unilamellar vesicles (LUV) were prepared as previously described. Briefly, lipids (1,2-dioleoyl-sn-glycero-3-phospho-L-serine (DOPS) and 1-palmitoyl-2-(9,10-dibromostearoyl)phosphatidylcholine (BrPC)) (Avanti Polar Lipid) were dissolved in chloroform while GT1b trisodium salt (Santa Cruz Biotechnology) was dissolved in methanol. The lipids (70/20/10 mol % BrPC/DOPS/GT1b) were mixed, dried under nitrogen gas, and then placed under vacuum for overnight. The dried lipids were rehydrated and subjected to 5-10 rounds of freezing and thawing cycles. Liposomes were prepared by extrusion through a 200 nm pore membrane using an Avanti Mini Extruder according to the manufacturer's instructions.

The H_{C}E-GT1b binding experiment was conducted by mixing 1 UM of H_{C}E or H_{C}E pre-incubated with 2 μM of G6 with 200 UM of liposomes. The protein-liposome mixture was then incubated in a buffer containing 100 mM NaCl and 20 mM HEPES (pH 7.0) at room temperature for 1 hour followed by spinning progressively at 4,000×, 9,000×, and 16,000×g for 30 min each. Supernatant and pellet were separated and analyzed by SDS-PAGE.

Pulldown assay: For the structure-based mutagenesis studies, pulldown assays were performed with NI²⁺-NTA resins in 1 ml buffer containing 50 mM Tris, pH 7.5, 400 mM NaCl, 20 mM imidazole, and 0.1% Tween-20. His-tagged G6, SV2, or SV2-G6^{AA} variants served as the baits, and H_{C}E (WT and variants) served as the preys. To prepare the pull down, SV2 (5 μg) or SV2-G6^{AA} (10 μg) were pre-incubated with Ni²⁺-NTA resins at 12° C. for 1 hour. After washing away the unbound proteins, the resins were mixed with H_{C}E (32 μg, ~2-fold molar excess over the bait) at 12° C. for 1 hour. The resins were then washed twice, and the bound proteins were released from the resins with 400 mM imidazole and subjected to SDS-PAGE.

To examine the interactions between H_{C}E and SV2A-G6^{AA} at various pH, the pull down assays were carried out using Strep-Tactin resins (IBA Lifesciences) in three different buffers: 50 mM Tris, pH 7.5, 400 mM NaCl, and 0.1% Tween-20, or 50 mM sodium acetate, pH 5.0 or 4.6, 400 mM NaCl, and 0.1% Tween-20. The His-tagged SV2A-G6^{AA} (10 μg) that was first biotinylated using EZ-Link NHS-PEG4-Biotin (Thermo Fisher Scientific) served as the bait and H_{C}E (32 μg) served as the prey. The pull down assays were carried out at 12° C. for 1 hour. The resins were then washed twice, and the bound proteins were released from the resins with 50 mM biotin and subjected to SDS-PAGE.

Protein melting assay: The thermal stability of H_{C}E or SV2-G6^{AA} variants were measured using a fluorescence-based thermal shift assay on a StepOne real-time PCR machine (Life Technologies). Each protein (~0.5 mg/ml) was mixed with the fluorescent dye SYPRO Orange (Sigma-Aldrich) and heated from 25° C. to 90° C. in a linear ramp. The midpoint of the protein-melting curve (Tm) was determined using the analysis software provided by the instrument manufacturer. Data obtained from three independent experiments were averaged to generate the bar graph.

Biolayer interferometry assay. The binding between H_{C}E and SV2A-G6^{AA}, SV2A^{Y535T/Y557G}-G6^{AA}, SV2C-G6^{AA}, and SV2C^{T521Y/D543Y}-G6^{AA} were examined by BLI assays using an OctetRED96 (ForteBio). Briefly, equal amounts of biotinylated SV2-G644 variants (400 nM) was immobilized onto the Dip and Read Streptavidin (SA) Biosensors (ForteBio) and balanced with the buffer (50 mM Tris, pH 7.5, 400 mM NaCl, 0.5% BSA, and 0.1% Tween 20). The biosensors were then exposed to 1 μM H_{C}E (binding phase), followed by washing with the buffer (dissociation phase).

Antibodies and constructs: The following antibodies were purchased from the indicated vendors: rabbit monoclonal antibody against β-actin (ABcional, AC038); mouse monoclonal antibodies against SNAP-25 (Synaptic systems, Cl 71.1) or Syt-1 (Synaptic systems, #105011); rabbit polyclonal antibody against SV2C (Synaptic systems, #119202). SV2 mouse monoclonal antibody (pan-SV2) was generously provided by E. Chapman (Madison, WI) and is available from Developmental Studies Hybridoma Bank (AB_2315387). Secondary antibodies were purchased from the following vendors: goat anti-rabbit-HRP (Bio-Rad, 1705046) and goat anti-mouse-HRP (Abcam, ab97023).

BoNT/E utilized for cell-based assays was purchased from Metabiologics or List Biologics (#141A) by the Dong lab. No recombinant BoNT/E was imported into the United States. All active BoNTs are stored in a locked freezer. Used toxins and contaminated media/reagents/containers are exposed to 10% bleach solution for decontamination. Lentiviral constructs (in Lox-Syn-Syn vector) encoding full-length rat SV2A and SV2C were previously described. Rat SV2Ac chimera was generated by replacing F487-E532 of SV2A with V473-K518 of SV2C using Gibson assembly and subcloned into Lox-Syn-Syn vector. SV2A (Y535T/Y557E) and SV2C (T521Y/E543Y) were generated by site-directed mutagenesis through overlapping PCR. All constructs were confirmed by sequencing (Genewiz).

Mouse lines and pregnant rats: Sv2a- and Sv2b-knockout mice (strain B6; 129P2-Sv2a^{tm1Sud}Sv2b^{tm1Sud}/J, stock No: 006383; cryo recovery) were obtained from the Jackson Laboratory. Mice heterozygous for both Sv2a and Sv2b were bred together to generate Sv2a+/−Sv2b−/− mice. Three primers were used to genotype Sv2a or 2b. Primers for Sv2a: mutant: GAG CGC GCG CGG CGG AGT TGT TGA C; wild type: GTT GAC TGA GAG TGA GAT GAG C; common: GAG TTA GGG ATG AGT GTT CTG G. Primers for Sv2b: mutant: GAG CGC GCG CGG CGG AGT TGT TGA C; wild type: TCA TCC AGA TGA TGT CAA GTC TAA GC; common: GGC ACT CAG CCA CTA ACT CTC AGT ACA). Once Sv2a+/−Sv2b−/− were established, they were bred to generate sv2a/sv2b double KO pups as mice homozygous for sv2a/sv2b double KO were not viable. Timed pregnant rats (Sprague Dawley strain) were purchased from Charles River.

Neuron culture and lentivirus transduction: Rat cortical neurons were prepared from E18-19 embryos dissected from pregnant rats. Mouse Sv2a/2b double KO neurons were prepared from postnatal day 1 pups as previously described. The pups were genotyped using Sv2a primers within 24 hours after the pups were born. Sv2a−/−Sv2b−/−pups were used to culture the neurons. Dissected cortex was digested with papain for 1 hour with tapping every 10-15 min, according to the manufacturer's instructions (Worthington Biochemical). Neurons were plated on poly-D-lysine-coated 24-well plates. Experiments were carried out generally with DIV (days in vitro) 13-15 neurons. Lentiviruses were prepared from HEK293T cells, as previously described. 2.5 UM of arabinosylcytosine C (AraC) was added to neurons at DIV4, while lentiviruses were added at DIV5-6.

H_{C}E binding to neurons: Neurons were exposed to 200 nM of biotinylated H_{C}E variants in high-K⁺ buffer containing 87 mM NaCl, 56 mM KCl, 1.5 mM KH₂PO₄, 8 mM Na₂HPO₄, 0.5 mM MgCl, and 1 mM CaCh, for 5 min at 37° C. Neurons were then washed three times with each 2.5 mL of phosphate-buffered saline (PBS). Neurons were harvested in a lysis buffer (PBS with 1% Triton X-100, 0.05% SDS and protease-inhibitor cocktail (Roche), 40 μL per well in 24-well plates). Lysates were centrifuged for 10 min at 4° C., and the supernatants were subjected to SDS-PAGE and western blot analysis. Binding of HE was detected with a Streptavidin-tagged horseradish peroxidase (HRP) (Cell signaling technology, 3999s), which recognizes biotinylated H_{C}E. All experiments were repeated three times independently.

Entry of BoNT/E into neurons: BoNT/E was pre-activated with trypsin (Sigma, Type XIII-TPCK treated) for 30 min at 37° C., and quenched with soybean trypsin inhibitor (Sigma, Type I-S, T6522, toxin: trypsin: inhibitor=10:1:10 in molar ratio). Neurons were exposed to 200 μM activated BoNT/E in medium. Cells were incubated for 14-20 hours at 37° C. Neuron lysates were then harvested and subjected to western blot analysis to detect cleavage of SNAP-25 through chemiluminescence (SuperSignal West Pico Plus, Thermo Scientific).

Mouse phrenic nerve hemidiaphragm assay. The MPN assay was performed employing 20-30 g swiss mice (Janvier SA, France) as described previously. Mice were euthanized by CO₂ anesthesia and subsequently exsanguinated. The phrenic nerve hemidiaphragm tissue was explanted, placed into an organ bath and continuously stimulated at 5-25 mA with a frequency of 1 Hz and a 0.1 ms pulse duration. Isometric contractions were transformed using a force transducer and recorded with VitroDat Online software (FMI GmbH, Germany). The time required to decrease the amplitude to 50% of the starting value (paralytic half-time) was determined. To allow comparison of the altered neurotoxicity of mutants with BoNT/E1 wild-type (displaying a specific activity of 0.41×10⁸ LD₅₀/mg), its MPN assay dose-response-curve logarithmic function (y (BoNT/E1 wild-type; 2.0, 4.0, 8.0 μM)=−23.61 In(x)+104.11, R2=0.999) consisting of three concentrations determined in 5-8 technical replicates as described previously was employed. Mean (n=3-5) of resulting paralytic half-times of the BoNT/E1 mutants were converted to concentrations of the wild-type employing the above function and finally expressed as relative neurotoxicity. Similar studies were performed to compare the potency of BoNT/E-V3.5 and the wild-type BoNT/E1 and BoNT/E3.

### Example 2

The following is a non-limiting example of the present invention. It is to be understood that said example is not intended to limit the present invention in any way. Equivalents or substitutes are within the scope of the present invention.

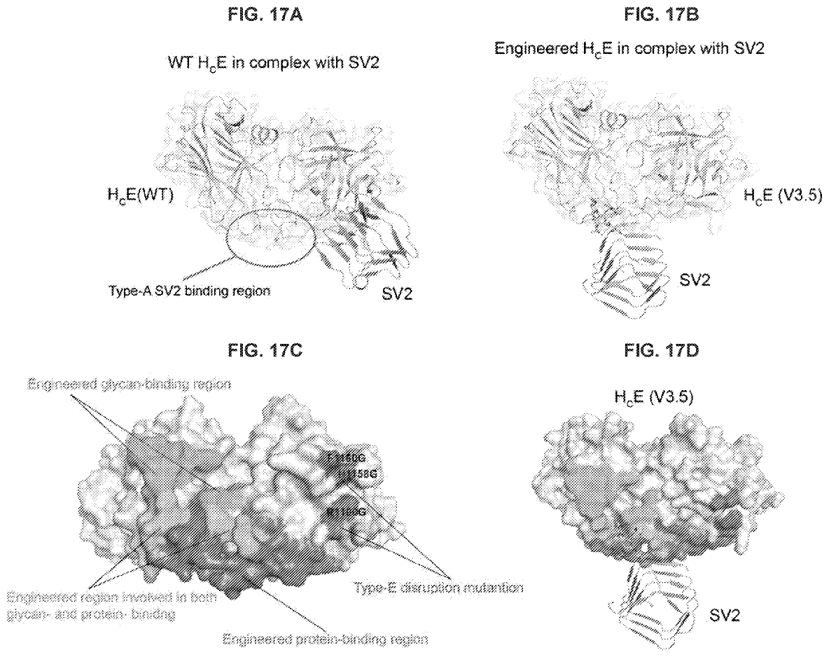

Structure-based protein engineering was performed to craft a new SV2-binding site onto H_{C}E, which is located at a site different from the endogenous SV2-binding site (FIG. 17A). This newly introduced SV2-binding site on H_{C}E mimics the SV2-binding mode of H_{C}A.

In early stage of the engineering (e.g. H_{C}E-V1, SEQ ID NO: 2), mutagenesis was focused on mutations that improved SV2-protein-mediated binding (bolded amino acid) and both protein/glycan-mediated binding (underlined).

Further optimizations were performed based on H_{C}E-V1. For example, in H_{C}E-V3.5 (SEQ ID NO: 3), SV2-protein-mediated binding was further improved (bolded amino acid), additional mutations were introduced to improve the SV2-glycan-mediated binding (italicized), and mutations that improve both protein/glycan-mediated binding were kept (underlined) (FIG. 17C).

In H_{C}E-V3.5, the endogenous SV2-binding site on HE was abolished by introducing three mutations, R1100G/H1158G/F1160G (bolded and underlined amino acid in SEQ ID NO: 3, FIG. 17C).

The 3D crystal structure was determined for H_{C}E-V3.5 in complex with SV2Ac, which demonstrates that SV2Ac binds at the newly engineered binding site on H_{C}E-V3.5, but not the endogenous binding site (FIGS. 17B and 17D). Furthermore, the interactions between HE-V3.5 and the protein- and glycan-moieties of SV2Ac are highly similar to that between H_{C}A and SV2, demonstrating that HE-V3.5 faithfully mimics H_{C}A for SV2 recognition.

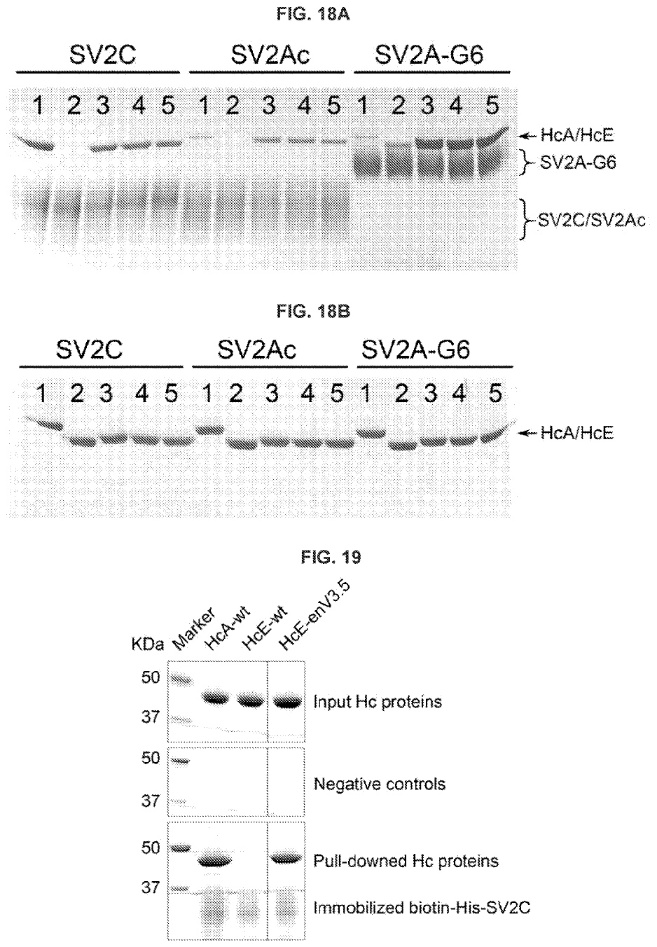

Interactions between H_{C}E-V1 and related variants and SV2 were examined using pull-down assays. Here, SV2C, SV2cA, and SV2A-G6 (G6^{AA}) were biotin-labeled and used as baits, which appear as a range of smear bands because they are glycosylated. Sample #1, wild-type HA; #2, wild-type H_{C}E; #3, H_{C}E-V1; #4, H_{C}E-V1-2G; #5, H_{C}E-V1-3G were used as preys. FIG. 18B shows that equal amounts of H_{C}A or H_{C}E variants were used in this assay. H_{C}E-V1-2G (#4) carries H1158G/F1160G mutations, while HE-V1-3G (#5) carries R1100G/H1158G/F1160G mutations. Wild-type HE did not bind SV2C, and binds weakly to SV2Ac and SV2A-G6, as expected. H_{C}E-V1 binds strongly to SV2C, SV2Ac, and SV2A-G6, and its binding to SV2Ac and SV2A-G6 is even better than H_{C}A (FIG. 18A). Furthermore, H_{C}E-V1-2G and H_{C}E-V1-3G that carry mutations to disrupt the endogenous SV2A-binding site on H_{C}E did not affect H_{C}E-V1 binding to SV2Ac or SV2A-G6, which demonstrates that HE-V1 in fact uses the newly engineered H-A-like binding interface to bind SV2, but not the endogenous SV2-binding site.

FIG. 19 shows H_{C}E-V3.5 has a gain-of-function to bind SV2C. In this pull-down assay, a biotin-labeled SV2C strongly pull down HA-WT and H_{C}E-V3.5, but not H_{C}E-WT.

We next examined whether the improved SV2-binding features of H_{C}E-V3.5 can lead to improved biological activity in the context of BoNT/E holotoxin. For this end, we designed a new BoNT/E holotoxin that is composed of the LC-H_{N} fragment of the wild-type BoNT/E (underlined amino acids in SEQ #4) and H_{C}E-V3.5 (bolded amino acids), which is termed BoNT/E-V3.5.

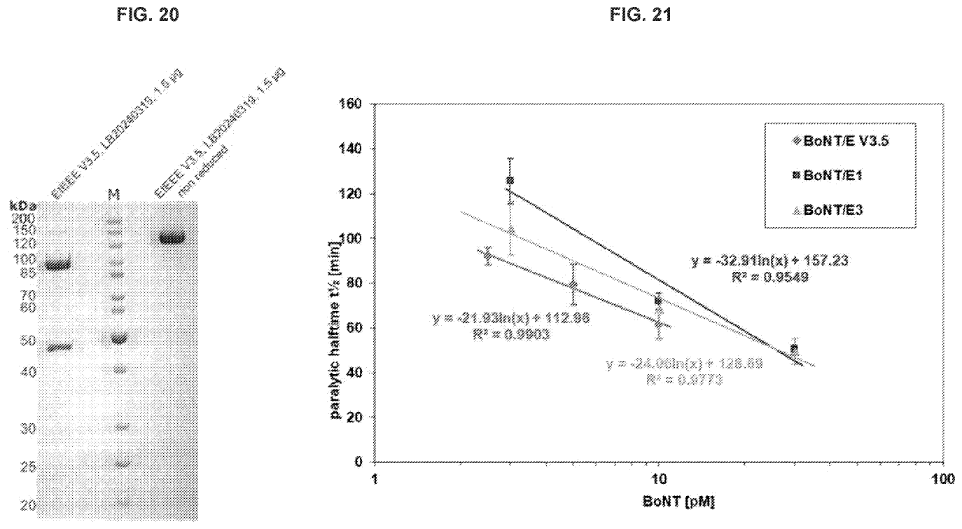

BoNT/E-V3.5 was recombinantly expressed and purified to high homogeneity as revealed by SDS-PAGE analysis (FIG. 20). Under the reducing condition (left sample), BoNT/E-V3.5 runs as two bands, where the upper band is the H_{N}-H_{C} domain and the lower band is the LC. Under the non-reducing condition (right sample), BoNT/E-V3.5 runs as a single band when the H_{N}-H_{C} domain and the LC are linked by a disulfide bond. This pattern of BoNT/E-V3.5 is identical to the well-documented feature of the wild-type BoNT/E.

The biological activity of BoNT/E-V3.5 was compared to the wild-type BoNT/E1 and BoNT/E3 by MPN hemidiaphragm assay (FIG. 21). BoNT/E-V3.5 was measured in three protein concentrations of 2.5, 5.0 and 10.0 μM in 4-5 biological replicates. Diagram shows mean SD. Comparison of BoNT/E-V3.5 with BoNT/E1 wild-type reveals an increase of 2.15-fold in biological activity and with BoNT/E3 wild-type an increase of 1.65-fold in biological activity.

### Example 3

The following is a non-limiting example of the present invention. It is to be understood that said example is not intended to limit the present invention in any way. Equivalents or substitutes are within the scope of the present invention.

A patient presents with recurrent migraines. Her physician prescribes a pharmaceutical formulation containing a modified serotype E botulinum neurotoxin (BoNT/E) comprising a plurality of mutations. The pharmaceutical formulation is administered via injection once every three months. After six months and two administrations, the patient experiences significant improvement, with a remarkable reduction in the frequency of migraine days. No side effects reported.

### Example 4

The following is a non-limiting example of the present invention. It is to be understood that said example is not intended to limit the present invention in any way. Equivalents or substitutes are within the scope of the present invention.

A surgeon employs a pharmaceutical formulation comprising a modified serotype E botulinum neurotoxin (BoNT/E) comprising a plurality of mutations to mitigate scar formation in a patient who has undergone excisions with linear repairs on the forehead. The surgeon precisely administers the pharmaceutical composition around the excision site. Upon follow-up a few days post-surgery, the patient's wound appears to be healing exceptionally well with reduced itch and pain. Remarkably, after just one month, the scar has completely vanished, leaving behind smooth and unblemished skin. No side effects reported.

### Example 5

The following is a non-limiting example of the present invention. It is to be understood that said example is not intended to limit the present invention in any way. Equivalents or substitutes are within the scope of the present invention.

A woman seeks treatment to diminish a few wrinkles between her eyebrows. An esthetician administers a single intramuscular injection of a composition comprising a modified serotype E botulinum neurotoxin (BoNT/E) comprising a plurality of mutations into the facial muscles underlying glabellar rhytids. This targeted approach effectively softens or entirely eradicates these lines. Treatment effect is observed as early as 24 hours following the injections and the effects last between 14 and 30 days. No side effects reported.

As used herein, the term “about” refers to plus or minus 10% of the referenced number. Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A modified serotype E botulinum neurotoxin (BoNT/E) comprising a plurality of mutations, wherein one or more mutations decrease endogenous BoNT/E receptor binding function; and wherein one or more mutations mimic endogenous botulinum neurotoxin A (BoNT/A) receptor binding.

2. The modified BoNT/E of claim 1, wherein the one or more mutations that decrease endogenous BoNT/E receptor binding function include a mutation that inhibits binding of BoNT/E to a receptor.

3. The modified BoNT/E of claim 2, wherein the mutation that inhibits binding of BoNT/E to the receptor comprises mutations at a protein interface, a glycan interface, or both.

4. The modified BoNT/E of claim 3, wherein the mutation at the protein interface comprises mutations at R1100, K1102, A1154, T1157, H1158, L1159, F1160, or a combination thereof.

5. The modified BoNT/E of claim 3, wherein the mutation at the glycan interface comprises mutations at Y879, Y881, Y891, R922, N988, Y1041, H1247, G1248, or a combination thereof.

6. The modified BoNT/E of claim 2, wherein the mutation that inhibits binding of BoNT/E to the receptor comprises mutations at R1100, H1158, F1160, or a combination thereof.

7. The modified BoNT/E of claim 6, wherein the mutation that inhibits binding of BoNT/E to the receptor comprises R1100G, H1158G, F1160G, or a combination thereof.

8. The modified BoNT/E of claim 1, wherein the one or more mutations that mimic the endogenous BoNT/A receptor binding comprise mutations at the BoNT/A-like protein interface, BoNT/A-like glycan interface, or a combination thereof.

9. The modified BoNT/E of claim 1, wherein the receptor comprises a synaptic vesicle glycoprotein 2 (SV2).

10. The modified BoNT/E of claim 1, wherein the plurality of mutations increases binding affinity of the modified BoNT/E compared to a wild type BoNT/E.

11. The modified BoNT/E of claim 1, wherein the plurality of mutations modulate receptor binding specificity of the modified BoNT/E compared to a wild type BoNT/E.

12. The modified BoNT/E of claim 1, wherein the plurality of mutations enhance the biological activity of the modified BoNT/E compared to a wild type BoNT/E.

13. The modified BoNT/E of claim 1, wherein the modified BoNT/E comprises a sequence according to SEQ ID NO: 2, SEQ ID NO: 3, or SEQ ID NO: 4.

14. A pharmaceutical composition comprising the modified BoNT/E according to claim 1.
